SEQUENCE LISTING (151 sequences)

SEQ ID NO: 1 - DNA, 4293 bp, Maize White Line Mosaic Virus
agaatacctcctggatctaaccaatccgtgagagttggccatggccttggctagaggtgttctctcccagcgcgtcgtgacggcggcagttgacgttacttttggtagtgttgactacagtgacccacgcattgtggcagcactgtgtgatgggggtttgaaggggcgggcgaccgtaaggcgtcaaattgtaactgcgctcaaatggctagtgatggtgctcacttggcccgtaaggatgcccgcgatggcgatcgtgtggtgtctgacatgggtagcactgatggtcactcgaaccaccaggaagatctgctgtgtcgttagcaggttgtactccgagtcctccgccttagtccgtgcatactggcgtgtgtacaataaaaggactagggccgtggcttgcactggcctggtgggttccctggcactgtacggccctgctgctgtgttggtgtgggtgtgtcttctagtggtgttcgtcttttgtacactaccggctgatgcccgatactacatcaaattggccaagaaaatacaggatgcttgggacgcggttgaggaggatgacagcatcaccccagccgctgatggtggaccactggaggttcgctccgggcggaaccggttcgcgtgccgactggcagcgagggcaatcagtcgtgtgggcttgttgaagcccactaaggcaaacgctctcgtgtaccagaaggttatcctcgacgagatgaaagtgctcaacgtccggttcggtgaccgagtacgagtgctgccacttgccgtggtcgcgtgtctggaacggcccgatgctgtggatagggttgagggggtcattgacgccctcacctgtctgcctggcagcctctagggaggccttgtccgccgtgaagggtgcgacaccgacactgaccgcacaaaatttgatctatcagcggttcagggggtgacacgcatggagggaatcacggtacggacagggacctcagccaaaggtgggagaacttggtactcgttcaactcaccggcaacgacatatgagtacattgtccacaactcatcacttaagaacgtagtcaggggacttgtcgagcgggtcttctgtgttgtggacaagaaaactggtgaactggtccggcccccaaaacctgttaaggggctattcaccaagaagctcggtgacgtcggtcaagtagtgagtcaactcgttggttattgcccccactggacacgtcaagaattcttggcgtcttacaatgggccgcgaaaagccagttacgagcgggctgcgctaacgctagacactctgcccttgcgtgaggaggatgcgcatctgagcacctttgtaaaggcggagaagatcaacgtcactctgaaacctgatcctgccccacgagtgattcagccgcgtggacagcggtacaacattgaggtgggaaggtttctgaaacccctggaaccacgcctaatgaaggcgatcgataagctgtgggggtccaccacagctattaaggggtacacggttgagagagtcggggctatcatgaatgagaaagctaacagatttcgtgagcctgtgtttgtgggtttagatgcctctcggtttgaccaacattgttctgccgaggcccttagatgggaacacagtgtttacaacgacatctttcgatctgagtatctcgcaacactcttacagtggcaggtcaacaatagagggactgcctacactaaagagggtactgtgagttacaaggtagaagggtgccgtatgtctggggacatgaacacgtcgatgggaaattatttaatcatgtcctgcttgatctatgccttttgccgggaagttagactgaaagcggaattggctaactgtggtgacgattgcgtgctgtttttggagaaagaggatcttcacaagcttggcactttaccgcagtggtttgtacgtatgggatatacgatgaaggtggaggagccggtgtatgaggtggagcacattgagttctgccaaatgcgccccattcgcacctccagaggatgggtcatggtcaggcgtccggacactgttctaacaaaggattgttgtgttgtcaggggaggaatgactgaggagcggttgaagggatggcttggtagtatgcgcgatggcggtctcagccttgctggggacgtacccatattgggtgccttctaccggtccttcccatcatacgcttctcaggaagcttccgagtacagcgccccacacaagttccgggcgggtaagcagtacggcgctgtcacagacgagagccggtattccttttggctggcgtttgggctcacacccgacgaccagcttgctgtggagagtgaattgtcaaagatggcgtttcatactcgtccggagcaaaaaggaccgtaccagccctcgctacttgactactgcactagaacctgaccagttcaccagccaccttgactactgcactagaacctgaccagttcaccagccatggcgaggaagaagcggagcaaccaggtacagacgggacagggagtgaggcgagcagcaggggctgtcattacagctcctgtagctaggacccgacaagtgagggcccggccacctaaggtcgaggcgttagcgggcggtggttttcgggtcacccatagggagttgatcactaccattgccaactcggctacataccaggcgaacgggggtattgctggattaaagtacaggatgaatccgacgtacggctccaccttgacgtggtgtccggccttggcatccaacttcgaccagtatgtcttccgcaaattgaccttggaatacgtgccgacgtgtgggacaacggagacggggagggtgggcatctggttcgatagggactctgaagatgacccgcctgctgaccgagtggaattggctagtatgggggtacttgtggagactgctccatggagcggtgtcacactacaggtacccacggacaacaccaagagattctgcctcggcgctggtggcaacacggatgccaaactgatagaccttggtcaaatcggttttagtacgtacgcgggagctgggacgaacgctgtcggtgatctattcgccgagtatgtcgtggatctacactgcccgcaaccgtctggcgcattagtccaaacgttgcgaatcactagtgctggggtgcgaggacctgaagttggaccactatactacaacatgacaaaggcagcaactctcattgacctgacgttcttcacaccaggcacatttctgatctcaataggctgcgcagctacttcgtatacttcggagctagtcctgggaggagccacgctgaactcacgaacactcactgccacaggagccgggttttccgggtcctttaacgtcactgtgaccaagcccttagatggcttacgcatacaaggaaccggattcggtgactgtatgacgtttgctgtccgcgcgagggtggccaactctgttactgtctagctgtggctggctggaggataagaagctaaccacttcatgtcgataatcagtcttgacggagagtttgattgtcctccttatcaacccacctcatcccgctttcacttcactcacaaaacgcgcaagtctgctatttgtatcggtccttctactttcggcaaattatggcgagtcccgagggctgggtattacaccccaaccgatgtgacctttgtggttacgccacatatctccgagaaagctggcgttatggcgactgtcaaactcatagacgcatccgacatgagcccatcccgagtgctgttcgagaccaaggcgttcaaccttggccatgggacggtactggaggggtctcaattgccgttttgcctgccaatcggggaatatcctatacacttcgaggtcacggtgtcacgatcacagtttcggggagaacggacaatgtactcaacatcactcgagtggcaaatgatgtgttctcccaccccgttatccagggttcgatctgtgttcgcggttgcgcaccaaccagtgttggatgcggtcccgaatttctcaatgaaaaccaaaaagaagtctagcgtcctgtccggtggtaagggtcaagcgacagaaaagaggattttggctggtggtggtacggcccggggagtggttcccccgggatgcgtagcgccagctgaaggaatcccagtaatcgccactatagaagaccactaggacagcatgtactccacgcttcggcggggctataaggagtacatgatacccccccctatctttcacccagcttgctggggtagccc

SEQ ID NO: 2 - DNA, 825 bp, Maize White Line Mosaic Virus
atggccttggctagaggtgttctctcccagcgcgtcgtgacggcggcagttgacgttacttttggtagtgttgactacagtgacccacgcattgtggcagcactgtgtgatgggggtttgaaggggcgggcgaccgtaaggcgtcaaattgtaactgcgctcaaatggctagtgatggtgctcacttggcccgtaaggatgcccgcgatggcgatcgtgtggtgtctgacatgggtagcactgatggtcactcgaaccaccaggaagatctgctgtgtcgttagcaggttgtactccgagtcctccgccttagtccgtgcatactggcgtgtgtacaataaaaggactagggccgtggcttgcactggcctggtgggttccctggcactgtacggccctgctgctgtgttggtgtgggtgtgtcttctagtggtgttcgtcttttgtacactaccggctgatgcccgatactacatcaaattggccaagaaaatacaggatgcttgggacgcggttgaggaggatgacagcatcaccccagccgctgatggtggaccactggaggttcgctccgggcggaaccggttcgcgtgccgactggcagcgagggcaatcagtcgtgtgggcttgttgaagcccactaaggcaaacgctctcgtgtaccagaaggttatcctcgacgagatgaaagtgctcaacgtccggttcggtgaccgagtacgagtgctgccacttgccgtggtcgcgtgtctggaacggcccgatgctgtggatagggttgagggggtcattgacgccctcacctgtctgcctggcagcctctag

SEQ ID NO: 3 - DNA, 2394 bp, Maize White Line Mosaic Virus
atggccttggctagaggtgttctctcccagcgcgtcgtgacggcggcagttgacgttacttttggtagtgttgactacagtgacccacgcattgtggcagcactgtgtgatgggggtttgaaggggcgggcgaccgtaaggcgtcaaattgtaactgcgctcaaatggctagtgatggtgctcacttggcccgtaaggatgcccgcgatggcgatcgtgtggtgtctgacatgggtagcactgatggtcactcgaaccaccaggaagatctgctgtgtcgttagcaggttgtactccgagtcctccgccttagtccgtgcatactggcgtgtgtacaataaaaggactagggccgtggcttgcactggcctggtgggttccctggcactgtacggccctgctgctgtgttggtgtgggtgtgtcttctagtggtgttcgtcttttgtacactaccggctgatgcccgatactacatcaaattggccaagaaaatacaggatgcttgggacgcggttgaggaggatgacagcatcaccccagccgctgatggtggaccactggaggttcgctccgggcggaaccggttcgcgtgccgactggcagcgagggcaatcagtcgtgtgggcttgttgaagcccactaaggcaaacgctctcgtgtaccagaaggttatcctcgacgagatgaaagtgctcaacgtccggttcggtgaccgagtacgagtgctgccacttgccgtggtcgcgtgtctggaacggcccgatgctgtggatagggttgagggggtcattgacgccctcacctgtctgcctggcagcctctagggaggccttgtccgccgtgaagggtgcgacaccgacactgaccgcacaaaatttgatctatcagcggttcagggggtgacacgcatggagggaatcacggtacggacagggacctcagccaaaggtgggagaacttggtactcgttcaactcaccggcaacgacatatgagtacattgtccacaactcatcacttaagaacgtagtcaggggacttgtcgagcgggtcttctgtgttgtggacaagaaaactggtgaactggtccggcccccaaaacctgttaaggggctattcaccaagaagctcggtgacgtcggtcaagtagtgagtcaactcgttggttattgcccccactggacacgtcaagaattcttggcgtcttacaatgggccgcgaaaagccagttacgagcgggctgcgctaacgctagacactctgcccttgcgtgaggaggatgcgcatctgagcacctttgtaaaggcggagaagatcaacgtcactctgaaacctgatcctgccccacgagtgattcagccgcgtggacagcggtacaacattgaggtgggaaggtttctgaaacccctggaaccacgcctaatgaaggcgatcgataagctgtgggggtccaccacagctattaaggggtacacggttgagagagtcggggctatcatgaatgagaaagctaacagatttcgtgagcctgtgtttgtgggtttagatgcctctcggtttgaccaacattgttctgccgaggcccttagatgggaacacagtgtttacaacgacatctttcgatctgagtatctcgcaacactcttacagtggcaggtcaacaatagagggactgcctacactaaagagggtactgtgagttacaaggtagaagggtgccgtatgtctggggacatgaacacgtcgatgggaaattatttaatcatgtcctgcttgatctatgccttttgccgggaagttagactgaaagcggaattggctaactgtggtgacgattgcgtgctgtttttggagaaagaggatcttcacaagcttggcactttaccgcagtggtttgtacgtatgggatatacgatgaaggtggaggagccggtgtatgaggtggagcacattgagttctgccaaatgcgccccattcgcacctccagaggatgggtcatggtcaggcgtccggacactgttctaacaaaggattgttgtgttgtcaggggaggaatgactgaggagcggttgaagggatggcttggtagtatgcgcgatggcggtctcagccttgctggggacgtacccatattgggtgccttctaccggtccttcccatcatacgcttctcaggaagcttccgagtacagcgccccacacaagttccgggcgggtaagcagtacggcgctgtcacagacgagagccggtattccttttggctggcgtttgggctcacacccgacgaccagcttgctgtggagagtgaattgtcaaagatggcgtttcatactcgtccggagcaaaaaggaccgtaccagccctcgctacttgactactgcactagaacctga

SEQ ID NO: 4 - DNA, 999 bp, Maize White Line Mosaic Virus
atggcgaggaagaagcggagcaaccaggtacagacgggacagggagtgaggcgagcagcaggggctgtcattacagctcctgtagctaggacccgacaagtgagggcccggccacctaaggtcgaggcgttagcgggcggtggttttcgggtcacccatagggagttgatcactaccattgccaactcggctacataccaggcgaacgggggtattgctggattaaagtacaggatgaatccgacgtacggctccaccttgacgtggtgtccggccttggcatccaacttcgaccagtatgtcttccgcaaattgaccttggaatacgtgccgacgtgtgggacaacggagacggggagggtgggcatctggttcgatagggactctgaagatgacccgcctgctgaccgagtggaattggctagtatgggggtacttgtggagactgctccatggagcggtgtcacactacaggtacccacggacaacaccaagagattctgcctcggcgctggtggcaacacggatgccaaactgatagaccttggtcaaatcggttttagtacgtacgcgggagctgggacgaacgctgtcggtgatctattcgccgagtatgtcgtggatctacactgcccgcaaccgtctggcgcattagtccaaacgttgcgaatcactagtgctggggtgcgaggacctgaagttggaccactatactacaacatgacaaaggcagcaactctcattgacctgacgttcttcacaccaggcacatttctgatctcaataggctgcgcagctacttcgtatacttcggagctagtcctgggaggagccacgctgaactcacgaacactcactgccacaggagccgggttttccgggtcctttaacgtcactgtgaccaagcccttagatggcttacgcatacaaggaaccggattcggtgactgtatgacgtttgctgtccgcgcgagggtggccaactctgttactgtctag

SEQ ID NO: 5 - DNA, 684 bp, Maize White Line Mosaic Virus
atgtcgataatcagtcttgacggagagtttgattgtcctccttatcaacccacctcatcccgctttcacttcactcacaaaacgcgcaagtctgctatttgtatcggtccttctactttcggcaaattatggcgagtcccgagggctgggtattacaccccaaccgatgtgacctttgtggttacgccacatatctccgagaaagctggcgttatggcgactgtcaaactcatagacgcatccgacatgagcccatcccgagtgctgttcgagaccaaggcgttcaaccttggccatgggacggtactggaggggtctcaattgccgttttgcctgccaatcggggaatatcctatacacttcgaggtcacggtgtcacgatcacagtttcggggagaacggacaatgtactcaacatcactcgagtggcaaatgatgtgttctcccaccccgttatccagggttcgatctgtgttcgcggttgcgcaccaaccagtgttggatgcggtcccgaatttctcaatgaaaaccaaaaagaagtctagcgtcctgtccggtggtaagggtcaagcgacagaaaagaggattttggctggtggtggtacggcccggggagtggttcccccgggatgcgtagcgccagctgaaggaatcccagtaatcgccactatagaagaccactag

SEQ ID NO: 6 - DNA, 417 bp, Maize White Line Mosaic Virus
atggcgagtcccgagggctgggtattacaccccaaccgatgtgacctttgtggttacgccacatatctccgagaaagctggcgttatggcgactgtcaaactcatagacgcatccgacatgagcccatcccgagtgctgttcgagaccaaggcgttcaaccttggccatgggacggtactggaggggtctcaattgccgttttgcctgccaatcggggaatatcctatacacttcgaggtcacggtgtcacgatcacagtttcggggagaacggacaatgtactcaacatcactcgagtggcaaatgatgtgttctcccaccccgttatccagggttcgatctgtgttcgcggttgcgcaccaaccagtgttggatgcggtcccgaatttctcaatgaaaaccaaaaagaagtctag

SEQ ID NO: 7 - DNA, 1168 bp, Maize White Line Mosaic Virus
gatatttctgctagaaagactcttaatcgttctgaacactttcttgaaagttgcggctgaccaccgtacaggaattctctcgcactagtcgggtttgaagcgcgggtgtatctaggagggtaagcctagagcataaattgtaactaccgcgaataaggtcatggccacccagctcacaacgagagctagaagggcaactcgggtttctcgtaagggatcccagcctgcttctaagcaggacgtgaaacaagttgtcaagtccatccttggacaaagcctggaacacaagagagctaacctactcctgcctcccaccgtggttaacactacagggaacatttactgcctgacgcagtttgtgattgagggcgacggcattagccaaaggaccggtcgtgtcattaacttggagcagatggtgttgcgctatcggcgcactctggacaccacatctgcaaactccgggttcctgcgctatatagtgttccttgatactcagaaccaaggcacacttccggcaataacggacgtgctgtcatcccttgacgtatcatctggatacgaggttctgaatgcacagcagaatagatttaagttcctacttgatgaggttgaatcactgtgtgccagtgctaccaacctatccaaggcctccactctgaccttcaatcagaaggtgcaggttcactatgggggcgctgctgatgcggcaacttcaaaccggcgcaatgccgtgttcttcttggagttgtctgacaaggttgccacggggcctcagacgcgcttgggtgtacagctcaagttcactgatgcctagtcattctctgagtgaccgcctacctggttggggtaagacaccaggaacccctctacgaaatgttcagtcggaagctgagaacctcccggtgcatactgacattgtgagggttcggtaggaagttggccaaaggtttccggatataagccacccggttactgtctaactatccccaaattcggccgtgtctgtcgaaagacagctataggatactctggtgaagccaggaaatgttggagcagggatgtttcagcggtccactggctagcccttgcatggttcttgcatggtcctatagcggtgatgtaacggattccatccactctattattagagctacacgccacaccc

SEQ ID NO: 8 - DNA, 657 bp, Maize White Line Mosaic Virus
atggccacccagctcacaacgagagctagaagggcaactcgggtttctcgtaagggatcccagcctgcttctaagcaggacgtgaaacaagttgtcaagtccatccttggacaaagcctggaacacaagagagctaacctactcctgcctcccaccgtggttaacactacagggaacatttactgcctgacgcagtttgtgattgagggcgacggcattagccaaaggaccggtcgtgtcattaacttggagcagatggtgttgcgctatcggcgcactctggacaccacatctgcaaactccgggttcctgcgctatatagtgttccttgatactcagaaccaaggcacacttccggcaataacggacgtgctgtcatcccttgacgtatcatctggatacgaggttctgaatgcacagcagaatagatttaagttcctacttgatgaggttgaatcactgtgtgccagtgctaccaacctatccaaggcctccactctgaccttcaatcagaaggtgcaggttcactatgggggcgctgctgatgcggcaacttcaaaccggcgcaatgccgtgttcttcttggagttgtctgacaaggttgccacggggcctcagacgcgcttgggtgtacagctcaagttcactgatgcctag

SEQ ID NO: 9 - DNA, 4421 bp, Johnsongrass chlorotic stripe mosaic virus
gagaatactggcagtgattgaccatcacggtgagttgtccagccatggataccggtattctctcgcggcgcatagtgactgctgaagttgactttcaatttggttctgttgactacagtgacccaagaatagtccacgcattatgcaccccgggtttgaaggagcgggcgaccttcgggcgtcaaattgttactgcgctcaaaatggccgtcattgcactgacgttacctgtgtggtggcccctcagacttgtctggagggtcatcatcatgggagtgctgtgggtcaccaggttcwtcactcggtgcaccaacctcatcaaatggtgcgttaaggagacgcgcgttaccgtgcgagcttattggaayattctcaacaagcgtgccagggggttggttgtactgggttgttgggcctcctttgtgttgtatggtccctatgccttacttttgtggctgggcgtgattgttggatacataatttgtgtcctaccgtctaatgtccgctactacattgagctgggccagaaaatacaggatgcatgggactctgtggaagcggatgataccatagaggctccgtgtaatggtgatatcctggaggttcgcaagggacgcaataagttcgcttgcaaactggctgcccgggcaattggtagagttggcttgctgaaggccacccctgctaatgccctggtctatcagaaagtgatcttggatgagatgaaaatcttaaatgttcgctttgctgatcgagttaggattttgccattagcagtgatggctagtcttgacaggccagacgccgtggctagggttgaggactgcgtggcagccctcacccaacgcggtgtgagcctctagggaggcctagtccgccgagagggttgtgacaccaccactgaccgcacaaattttgatttatcagcggttaaaggggtgggtcccacagagggactctcggtgagggctgggacctcggccaagggtgatagaagttggtactccttcaactcactggccactacatatgagtacgttgttcacaacggttccttgaaaaacgtgtgcagaggacttgtcgagcgggtcttctgtgttgtggacaagcaaagcgggaaattggtccgccccccaaaaccgaagccgggggtcttttccgctaagctcggtgacgttggtcgaactgtgagctcaatcgttggttattgcccccactggacacgtgacgagtttgttgcgtcttacagtgggccgcgaaaagcctcatacgagcgagctgcacagacgctagacactctacctctcatggaaagtgatgcacacttgagcacctttgtgaaggcagagaagatcaatgtcacgttgaagcccgacccggccccgcgtgtgatacaaccacggggccagcgatataacattgaggtcgggcggtttttgaagcccctggaaccacgtctcatgaaggcgatagataaactgtggggatccaccacagctattaaggggtatacggttgagaaggtcggctcgatctttgcagataaggcttcaaggtttaggcacccggtctatgttgggcttgatgcttcccgctttgaccagcactgtagtgctgatgcgttaaggtgggaacattctgtctacaatgatatattccgctcgccttacttagccgagctcctggaatggcaggtccacaatcgtgggtcagcctacacccacgagggcaaggttaattatagggtggaggggtgtcggatgtctggggacatgaacacttccatgggaaactatctgattatgtcatgtctcatatatcagttctgcaaggaaatcgggttgcacgcggagctagcaaactgtggtgatgattgtgtgctattcctggagaaacatgatcttaagaagcttaagcacttaccgcagtggtttgttaaaatgggatatactatgaaggttgaatcaccggtgtacgaacttgaggaagttgaattctgtcagatgcacccggtgagaacctctagggggtgggtgatggttaggcggcctgacacggtcatgactaaggactgttgtgtcgtcaggggaggaatgacaacggagcggctgcgagggtggttgggtgcgatgagagatggggggttgagcctagccggcgatgttcctgttctctcagcgttttattcttcattccctcaataccgcaacggagaaacctctgattatgatgcaccacacaagttcagggcgggtaagcagtatggtgctatcacggctgaggcacggtattcattctggctggcgttcgggttaacacctgatgatcagctagctattgaaggggacctttcatccttcaagttttcacttgaaccacaggatttggtcacctccatgcccagcttacttgactactgcactagaacctgaccagttcaccctaacacgatgtcgatcgtcccagcgaatacgaacagagccctagtgcgcgcaggcactgctcttgcctcaggagccatgacagccatggttccctatgccgccgcaggcgcccaccagattgggcaacgcctggggaagaaggtgtggaacgggtgggttgggtttccagggggcctggaaccgcctcagaaaaaggatgacgaacgggggaggagttcccatgattgttggaagtggaggtgggactgtggctgcaccagtcgctgtatcccgccaaatccgcagcaggaagccgaagttcacaagtgtcaaaggtcaagtgagagtgactcatcgtgagtatgttacccaagtctccggggtgggctccggattgttccagctcaatggaggattgccatcaggccagtttagggttaacccaaacaatgctgcctgcttcccgtggttgctaagcatagcatcgaacttcgaccagtacagatttgttaatctgcagctgtgttatgttccgctgtgcgccacaacggaggtggggcgagtggctctcttctacgacaaggacagcggagatagtgggccgtttgagcgagctgagcttgccaacatgacccattgtgccgaaacaccaccatgggcagaggtatcactcacagttccgtgcgacaatgtcaagcggtacctgaatgattccaatgttactgaccttaagctcgttgacgccggacggttcggttacgcggtgtatgggggtaatgccaatacctatggcgatctcttcatacaatacaccgtagaacttagtgagccacagcctacggctggactcattggggaggtamctggtaatgccggtacggtggcaggcgtcgtgcaacctgcgtacttcaactttgatggattctccacaacccaagtagcattcaagcctaccgtcgtgggtacatatctcatgacgttcatacttgacggcacaggtctggtgttgggcaatgtcacatcctctgctcctgaggggatgtctgtcctggaccagaatgtagcaggatcagccacacgtgtcatctatgtgtgcagggttaccgtccagcggccaggcgaccggttgttcttcaattacaccggcacagccaccttctggaacttattcgtggtgcgtgctacgagagacatctctatcaccacctagtcgcgtcgtgcctgggggattagaagctgaccacttcaatgtctatcgtcaatatcgacggtgagtttgagcagcctcaattccaggataccccttcgaaagtctacatttcccataaatctcgcaagtctctagtgtgcttggggccatctgtcttccacaagttatggaaggtcccaaagactgggttttacacccccaccggtgtgacttttgtggtcacgccacatatctccgagagtgctggcgtcacggcagtgatcaagttaatcgacatgagcgacatgagcccttcccgcgtcttgtacaagtccaaggagttcaacctgggacatggcctgacattggaagggtcacaactgccgttttgcctgccaatcggggagtatcctatacacttcgaggtcacggtgtcacgatcacagtttcaggccacgagaacgatgttttcaacgtcgctcgagtggcatctgatgtactcacccaccccgttatccagggtgagatctgtgttcggggtagcccaccaaccggtgttggaggtggaaaccaacttccgtatgaaaaccaaacaaatatcgtctagcgtcgtcgctgtgttgccgaagcagaaagccctaggaaagggcctaaagcctgttggtggtacgactcctggtttggtcaccgggaactgcgtaggaacagactgaaggtcactagtactggcactatggcggcataagcgacacacggagccacacttcggtggggctataaggttccgtgttgtatccctcttactttca

SEQ ID NO: 10 - DNA, 819 bp, Johnsongrass chlorotic stripe mosaic virus
atggataccggtattctctcgcggcgcatagtgactgctgaagttgactttcaatttggttctgttgactacagtgacccaagaatagtccacgcattatgcaccccgggtttgaaggagcgggcgaccttcgggcgtcaaattgttactgcgctcaaaatggccgtcattgcactgacgttacctgtgtggtggcccctcagacttgtctggagggtcatcatcatgggagtgctgtgggtcaccaggttcwtcactcggtgcaccaacctcatcaaatggtgcgttaaggagacgcgcgttaccgtgcgagcttattggaayattctcaacaagcgtgccagggggttggttgtactgggttgttgggcctcctttgtgttgtatggtccctatgccttacttttgtggctgggcgtgattgttggatacataatttgtgtcctaccgtctaatgtccgctactacattgagctgggccagaaaatacaggatgcatgggactctgtggaagcggatgataccatagaggctccgtgtaatggtgatatcctggaggttcgcaagggacgcaataagttcgcttgcaaactggctgcccgggcaattggtagagttggcttgctgaaggccacccctgctaatgccctggtctatcagaaagtgatcttggatgagatgaaaatcttaaatgttcgctttgctgatcgagttaggattttgccattagcagtgatggctagtcttgacaggccagacgccgtggctagggttgaggactgcgtggcagccctcacccaacgcggtgtgagcctctag

SEQ ID NO: 11 - DNA, 2388 bp, Johnsongrass chlorotic stripe mosaic virus
atggataccggtattctctcgcggcgcatagtgactgctgaagttgactttcaatttggttctgttgactacagtgacccaagaatagtccacgcattatgcaccccgggtttgaaggagcgggcgaccttcgggcgtcaaattgttactgcgctcaaaatggccgtcattgcactgacgttacctgtgtggtggcccctcagacttgtctggagggtcatcatcatgggagtgctgtgggtcaccaggttcwtcactcggtgcaccaacctcatcaaatggtgcgttaaggagacgcgcgttaccgtgcgagcttattggaayattctcaacaagcgtgccagggggttggttgtactgggttgttgggcctcctttgtgttgtatggtccctatgccttacttttgtggctgggcgtgattgttggatacataatttgtgtcctaccgtctaatgtccgctactacattgagctgggccagaaaatacaggatgcatgggactctgtggaagcggatgataccatagaggctccgtgtaatggtgatatcctggaggttcgcaagggacgcaataagttcgcttgcaaactggctgcccgggcaattggtagagttggcttgctgaaggccacccctgctaatgccctggtctatcagaaagtgatcttggatgagatgaaaatcttaaatgttcgctttgctgatcgagttaggattttgccattagcagtgatggctagtcttgacaggccagacgccgtggctagggttgaggactgcgtggcagccctcacccaacgcggtgtgagcctctagggaggcctagtccgccgagagggttgtgacaccaccactgaccgcacaaattttgatttatcagcggttaaaggggtgggtcccacagagggactctcggtgagggctgggacctcggccaagggtgatagaagttggtactccttcaactcactggccactacatatgagtacgttgttcacaacggttccttgaaaaacgtgtgcagaggacttgtcgagcgggtcttctgtgttgtggacaagcaaagcgggaaattggtccgccccccaaaaccgaagccgggggtcttttccgctaagctcggtgacgttggtcgaactgtgagctcaatcgttggttattgcccccactggacacgtgacgagtttgttgcgtcttacagtgggccgcgaaaagcctcatacgagcgagctgcacagacgctagacactctacctctcatggaaagtgatgcacacttgagcacctttgtgaaggcagagaagatcaatgtcacgttgaagcccgacccggccccgcgtgtgatacaaccacggggccagcgatataacattgaggtcgggcggtttttgaagcccctggaaccacgtctcatgaaggcgatagataaactgtggggatccaccacagctattaaggggtatacggttgagaaggtcggctcgatctttgcagataaggcttcaaggtttaggcacccggtctatgttgggcttgatgcttcccgctttgaccagcactgtagtgctgatgcgttaaggtgggaacattctgtctacaatgatatattccgctcgccttacttagccgagctcctggaatggcaggtccacaatcgtgggtcagcctacacccacgagggcaaggttaattatagggtggaggggtgtcggatgtctggggacatgaacacttccatgggaaactatctgattatgtcatgtctcatatatcagttctgcaaggaaatcgggttgcacgcggagctagcaaactgtggtgatgattgtgtgctattcctggagaaacatgatcttaagaagcttaagcacttaccgcagtggtttgttaaaatgggatatactatgaaggttgaatcaccggtgtacgaacttgaggaagttgaattctgtcagatgcacccggtgagaacctctagggggtgggtgatggttaggcggcctgacacggtcatgactaaggactgttgtgtcgtcaggggaggaatgacaacggagcggctgcgagggtggttgggtgcgatgagagatggggggttgagcctagccggcgatgttcctgttctctcagcgttttattcttcattccctcaataccgcaacggagaaacctctgattatgatgcaccacacaagttcagggcgggtaagcagtatggtgctatcacggctgaggcacggtattcattctggctggcgttcgggttaacacctgatgatcagctagctattgaaggggacctttcatccttcaagttttcacttgaaccacaggatttggtcacctccatgcccagcttacttgactactgcactagaacctga

SEQ ID NO: 12 - DNA, 1095 bp, Johnsongrass chlorotic stripe mosaic virus
atgccgccgcaggcgcccaccagattgggcaacgcctggggaagaaggtgtggaacgggtgggttgggtttccagggggcctggaaccgcctcagaaaaaggatgacgaacgggggaggagttcccatgattgttggaagtggaggtgggactgtggctgcaccagtcgctgtatcccgccaaatccgcagcaggaagccgaagttcacaagtgtcaaaggtcaagtgagagtgactcatcgtgagtatgttacccaagtctccggggtgggctccggattgttccagctcaatggaggattgccatcaggccagtttagggttaacccaaacaatgctgcctgcttcccgtggttgctaagcatagcatcgaacttcgaccagtacagatttgttaatctgcagctgtgttatgttccgctgtgcgccacaacggaggtggggcgagtggctctcttctacgacaaggacagcggagatagtgggccgtttgagcgagctgagcttgccaacatgacccattgtgccgaaacaccaccatgggcagaggtatcactcacagttccgtgcgacaatgtcaagcggtacctgaatgattccaatgttactgaccttaagctcgttgacgccggacggttcggttacgcggtgtatgggggtaatgccaatacctatggcgatctcttcatacaatacaccgtagaacttagtgagccacagcctacggctggactcattggggaggtamctggtaatgccggtacggtggcaggcgtcgtgcaacctgcgtacttcaactttgatggattctccacaacccaagtagcattcaagcctaccgtcgtgggtacatatctcatgacgttcatacttgacggcacaggtctggtgttgggcaatgtcacatcctctgctcctgaggggatgtctgtcctggaccagaatgtagcaggatcagccacacgtgtcatctatgtgtgcagggttaccgtccagcggccaggcgaccggttgttcttcaattacaccggcacagccaccttctggaacttattcgtggtgcgtgctacgagagacatctctatcaccacctag

SEQ ID NO: 13 - DNA, 654 bp, Johnsongrass chlorotic stripe mosaic virus
atgtctatcgtcaatatcgacggtgagtttgagcagcctcaattccaggataccccttcgaaagtctacatttcccataaatctcgcaagtctctagtgtgcttggggccatctgtcttccacaagttatggaaggtcccaaagactgggttttacacccccaccggtgtgacttttgtggtcacgccacatatctccgagagtgctggcgtcacggcagtgatcaagttaatcgacatgagcgacatgagcccttcccgcgtcttgtacaagtccaaggagttcaacctgggacatggcctgacattggaagggtcacaactgccgttttgcctgccaatcggggagtatcctatacacttcgaggtcacggtgtcacgatcacagtttcaggccacgagaacgatgttttcaacgtcgctcgagtggcatctgatgtactcacccaccccgttatccagggtgagatctgtgttcggggtagcccaccaaccggtgttggaggtggaaaccaacttccgtatgaaaaccaaacaaatatcgtctagcgtcgtcgctgtgttgccgaagcagaaagccctaggaaagggcctaaagcctgttggtggtacgactcctggtttggtcaccgggaactgcgtaggaacagactga

SEQ ID NO: 14 - DNA, 420 bp, Johnsongrass chlorotic stripe mosaic virus
atggaaggtcccaaagactgggttttacacccccaccggtgtgacttttgtggtcacgccacatatctccgagagtgctggcgtcacggcagtgatcaagttaatcgacatgagcgacatgagcccttcccgcgtcttgtacaagtccaaggagttcaacctgggacatggcctgacattggaagggtcacaactgccgttttgcctgccaatcggggagtatcctatacacttcgaggtcacggtgtcacgatcacagtttcaggccacgagaacgatgttttcaacgtcgctcgagtggcatctgatgtactcacccaccccgttatccagggtgagatctgtgttcggggtagcccaccaaccggtgttggaggtggaaaccaacttccgtatgaaaaccaaacaaatatcgtctag

SEQ ID NO: 15 - DNA, 6629 bp, Artificial Sequence
  Probe
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccagaatacctcctggatctaaccaatccgtgagagttggccatggccttggctagaggtgttctctcccagcgcgtcgtgacggcggcagttgacgttacttttggtagtgttgactacagtgacccacgcattgtggcagcactgtgtgatgggggtttgaaggggcgggcgaccgtaaggcgtcaaattgtaactgcgctcaaatggctagtgatggtgctcacttggcccgtaaggatgcccgcgatggcgatcgtgtggtgtctgacatgggtagcactgatggtcactcgaaccaccaggaagatctgctgtgtcgttagcaggttgtactccgagtcctccgccttagtccgtgcatactggcgtgtgtacaataaaaggactagggccgtggcttgcactggcctggtgggttccctggcactgtacggccctgctgctgtgttggtgtgggtgtgtcttctagtggtgttcgtcttttgtacactaccggctgatgcccgatactacatcaaattggccaagaaaatacaggatgcttgggacgcggttgaggaggatgacagcatcaccccagccgctgatggtggaccactggaggttcgctccgggcggaaccggttcgcgtgccgactggcagcgagggcaatcagtcgtgtgggcttgttgaagcccactaaggcaaacgctctcgtgtaccagaaggttatcctcgacgagatgaaagtgctcaacgtccggttcggtgaccgagtacgagtgctgccacttgccgtggtcgcgtgtctggaacggcccgatgctgtggatagggttgagggggtcattgacgccctcacctgtctgcctggcagcctctagggaggccttgtccgccgtgaagggtgcgacaccgacactgaccgcacaaaatttgatctatcagcggttcagggggtgacacgcatggagggaatcacggtacggacagggacctcagccaaaggtgggagaacttggtactcgttcaactcaccggcaacgacatatgagtacattgtccacaactcatcacttaagaacgtagtcaggggacttgtcgagcgggtcttctgtgttgtggacaagaaaactggtgaactggtccggcccccaaaacctgttaaggggctattcaccaagaagctcggtgacgtcggtcaagtagtgagtcaactcgttggttattgcccccactggacacgtcaagaattcttggcgtcttacaatgggccgcgaaaagccagttacgagcgggctgcgctaacgctagacactctgcccttgcgtgaggaggatgcgcatctgagcacctttgtaaaggcggagaagatcaacgtcactctgaaacctgatcctgccccacgagtgattcagccgcgtggacagcggtacaacattgaggtgggaaggtttctgaaacccctggaaccacgcctaatgaaggcgatcgataagctgtgggggtccaccacagctattaaggggtacacggttgagagagtcggggctatcatgaatgagaaagctaacagatttcgtgagcctgtgtttgtgggtttagatgcctctcggtttgaccaacattgttctgccgaggcccttagatgggaacacagtgtttacaacgacatctttcgatctgagtatctcgcaacactcttacagtggcaggtcaacaatagagggactgcctacactaaagagggtactgtgagttacaaggtagaagggtgccgtatgtctggggacatgaacacgtcgatgggaaattatttaatcatgtcctgcttgatctatgccttttgccgggaagttagactgaaagcggaattggctaactgtggtgacgattgcgtgctgtttttggagaaagaggatcttcacaagcttggcactttaccgcagtggtttgtacgtatgggatatacgatgaaggtggaggagccggtgtatgaggtggagcacattgagttctgccaaatgcgccccattcgcacctccagaggatgggtcatggtcaggcgtccggacactgttctaacaaaggattgttgtgttgtcaggggaggaatgactgaggagcggttgaagggatggcttggtagtatgcgcgatggcggtctcagccttgctggggacgtacccatattgggtgccttctaccggtccttcccatcatacgcttctcaggaagcttccgagtacagcgccccacacaagttccgggcgggtaagcagtacggcgctgtcacagacgagagccggtattccttttggctggcgtttgggctcacacccgacgaccagcttgctgtggagagtgaattgtcaaagatggcgtttcatactcgtccggagcaaaaaggaccgtaccagccctcgctacttgactactgcactagaacctgaccagttcaccagccaccttgactactgcactagaacctgaccagttcaccagccatggcgaggaagaagcggagcaaccaggtacagacgggacagggagtgaggcgagcagcaggggctgtcattacagctcctgtagctaggacccgacaagtgagggcccggccacctaaggtcgaggcgttagcgggcggtggttttcgggtcacccatagggagttgatcactaccattgccaactcggctacataccaggcgaacgggggtattgctggattaaagtacaggatgaatccgacgtacggctccaccttgacgtggtgtccggccttggcatccaacttcgaccagtatgtcttccgcaaattgaccttggaatacgtgccgacgtgtgggacaacggagacggggagggtgggcatctggttcgatagggactctgaagatgacccgcctgctgaccgagtggaattggctagtatgggggtacttgtggagactgctccatggagcggtgtcacactacaggtacccacggacaacaccaagagattctgcctcggcgctggtggcaacacggatgccaaactgatagaccttggtcaaatcggttttagtacgtacgcgggagctgggacgaacgctgtcggtgatctattcgccgagtatgtcgtggatctacactgcccgcaaccgtctggcgcattagtccaaacgttgcgaatcactagtgctggggtgcgaggacctgaagttggaccactatactacaacatgacaaaggcagcaactctcattgacctgacgttcttcacaccaggcacatttctgatctcaataggctgcgcagctacttcgtatacttcggagctagtcctgggaggagccacgctgaactcacgaacactcactgccacaggagccgggttttccgggtcctttaacgtcactgtgaccaagcccttagatggcttacgcatacaaggaaccggattcggtgactgtatgacgtttgctgtccgcgcgagggtggccaactctgttactgtctagctgtggctggctggaggataagaagctaaccacttcatgtcgataatcagtcttgacggagagtttgattgtcctccttatcaacccacctcatcccgctttcacttcactcacaaaacgcgcaagtctgctatttgtatcggtccttctactttcggcaaattatggcgagtcccgagggctgggtattacaccccaaccgatgtgacctttgtggttacgccacatatctccgagaaagctggcgttatggcgactgtcaaactcatagacgcatccgacatgagcccatcccgagtgctgttcgagaccaaggcgttcaaccttggccatgggacggtactggaggggtctcaattgccgttttgcctgccaatcggggaatatcctatacacttcgaggtcacggtgtcacgatcacagtttcggggagaacggacaatgtactcaacatcactcgagtggcaaatgatgtgttctcccaccccgttatccagggttcgatctgtgttcgcggttgcgcaccaaccagtgttggatgcggtcccgaatttctcaatgaaaaccaaaaagaagtctagcgtcctgtccggtggtaagggtcaagcgacagaaaagaggattttggctggtggtggtacggcccggggagtggttcccccgggatgcgtagcgccagctgaaggaatcccagtaatcgccactatagaagaccactaggacagcatgtactccacgcttcggcggggctataaggagtacatgatacccccccctatctttcacccagcttgctggggtagcccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaa

SEQ ID NO: 16 - DNA, 3506 bp, Artificial Sequence
  Probe
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctagggatatttctgctagaaagactcttaatcgttctgaacactttcttgaaagttgcggctgaccaccgtacaggaattctctcgcactagtcgggtttgaagcgcgggtgtatctaggagggtaagcctagagcataaattgtaactaccgcgaataaggtcatggccacccagctcacaacgagagctagaagggcaactcgggtttctcgtaagggatcccagcctgcttctaagcaggacgtgaaacaagttgtcaagtccatccttggacaaagcctggaacacaagagagctaacctactcctgcctcccaccgtggttaacactacagggaacatttactgcctgacgcagtttgtgattgagggcgacggcattagccaaaggaccggtcgtgtcattaacttggagcagatggtgttgcgctatcggcgcactctggacaccacatctgcaaactccgggttcctgcgctatatagtgttccttgatactcagaaccaaggcacacttccggcaataacggacgtgctgtcatcccttgacgtatcatctggatacgaggttctgaatgcacagcagaatagatttaagttcctacttgatgaggttgaatcactgtgtgccagtgctaccaacctatccaaggcctccactctgaccttcaatcagaaggtgcaggttcactatgggggcgctgctgatgcggcaacttcaaaccggcgcaatgccgtgttcttcttggagttgtctgacaaggttgccacggggcctcagacgcgcttgggtgtacagctcaagttcactgatgcctagtcattctctgagtgaccgcctacctggttggggtaagacaccaggaacccctctacgaaatgttcagtcggaagctgagaacctcccggtgcatactgacattgtgagggttcggtaggaagttggccaaaggtttccggatataagccacccggttactgtctaactatccccaaattcggccgtgtctgtcgaaagacagctataggatactctggtgaagccaggaaatgttggagcagggatgtttcagcggtccactggctagcccttgcatggttcttgcatggtcctatagcggtgatgtaacggattccatccactctattattagagctacacgccacacccgagctcgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaa

SEQ ID NO: 17 - DNA, 6629 bp, Artificial Sequence
  Probe
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccagaatacctcctggatctaaccaatccgtgagagttggccatggccttggctagaggtgttctctcccagcgcgtcgtgacggcggcagttgacgttacttttggtagtgttgactacagtgacccacgcattgtggcagcactgtgtgatgggggtttgaaggggcgggcgaccgtaaggcgtcaaattgtaactgcgctcaaatggctagtgatggtgctcacttggcccgtaaggatgcccgcgatggcgatcgtgtggtgtctgacatgggtagcactgatggtcactcgaaccaccaggaagatctgctgtgtcgttagcaggttgtactccgagtcctccgccttagtccgtgcatactggcgtgtgtacaataaaaggactagggccgtggcttgcactggcctggtgggttccctggcactgtacggccctgctgctgtgttggtgtgggtgtgtcttctagtggtgttcgtcttttgtacactaccggctgatgcccgatactacatcaaattggccaagaaaatacaggatgcttgggacgcggttgaggaggatgacagcatcaccccagccgctgatggtggaccactggaggttcgctccgggcggaaccggttcgcgtgccgactggcagcgagggcaatcagtcgtgtgggcttgttgaagcccactaaggcaaacgctctcgtgtaccagaaggttatcctcgacgagatgaaagtgctcaacgtccggttcggtgaccgagtacgagtgctgccacttgccgtggtcgcgtgtctggaacggcccgatgctgtggatagggttgagggggtcattgacgccctcacctgtctgcctggcagcctctagggaggccttgtccgccgtgaagggtgcgacaccgacactgaccgcacaaaatttgatctatcagcggttcagggggtgacacgcatggagggaatcacggtacggacagggacctcagccaaaggtgggagaacttggtactcgttcaactcaccggcaacgacatatgagtacattgtccacaactcatcacttaagaacgtagtcaggggacttgtcgagcgggtcttctgtgttgtggacaagaaaactggtgaactggtccggcccccaaaacctgttaaggggctattcaccaagaagctcggtgacgtcggtcaagtagtgagtcaactcgttggttattgcccccactggacacgtcaagaattcttggcgtcttacaatgggccgcgaaaagccagttacgagcgggctgcgctaacgctagacactctgcccttgcgtgaggaggatgcgcatctgagcacctttgtaaaggcggagaagatcaacgtcactctgaaacctgatcctgccccacgagtgattcagccgcgtggacagcggtacaacattgaggtgggaaggtttctgaaacccctggaaccacgcctaatgaaggcgatcgataagctgtgggggtccaccacagctattaaggggtacacggttgagagagtcggggctatcatgaatgagaaagctaacagatttcgtgagcctgtgtttgtgggtttagatgcctctcggtttgaccaacattgttctgccgaggcccttagatgggaacacagtgtttacaacgacatctttcgatctgagtatctcgcaacactcttacagtggcaggtcaacaatagagggactgcctacactaaagagggtactgtgagttacaaggtagaagggtgccgtatgtctggggacatgaacacgtcgatgggaaattatttaatcatgtcctgcttgatctatgccttttgccgggaagttagactgaaagcggaattggctaactgtggtgacgattgcgtgctgtttttggagaaagaggatcttcacaagcttggcactttaccgcagtggtttgtacgtatgggatatacgatgaaggtggaggagccggtgtatgaggtggagcacattgagttctgccaaatgcgccccattcgcacctccagaggatgggtcatggtcaggcgtccggacactgttctaacaaaggattgttgtgttgtcaggggaggaatgactgaggagcggttgaagggatggcttggtagtatgcgcgatggcggtctcagccttgctggggacgtacccatattgggtgccttctaccggtccttcccatcatacgcttctcaggaagcttccgagtacagcgccccacacaagttccgggcgggtaagcagtacggcgctgtcacagacgagagccggtattccttttggctggcgtttgggctcacacccgacgaccagcttgctgtggagagtgaattgtcaaagatggcgtttcatactcgtccggagcaaaaaggaccgtaccagccctcgctacttgactactgcactagaacctgaccagttcaccagccaccttgactactgcactagaacctgaccagttcaccagccatggcgaggaagaagcggagcaaccaggtacagacgggacagggagtgaggcgagcagcaggggctgtcattacagctcctgtagctaggacccgacaagtgagggcccggccacctaaggtcgaggcgttagcgggcggtggttttcgggtcacccatagggagttgatcactaccattgccaactcggctacataccaggcgaacgggggtattgctggattaaagtacaggatgaatccgacgtacggctccaccttgacgtggtgtccggccttggcatccaacttcgaccagtatgtcttccgcaaattgaccttggaatacgtgccgacgtgtgggacaacggagacggggagggtgggcatctggttcgatagggactctgaagatgacccgcctgctgaccgagtggaattggctagtatgggggtacttgtggagactgctccatggagcggtgtcacactacaggtacccacggacaacaccaagagattctgcctcggcgctggtggcaacacggatgccaaactgatagaccttggtcaaatcggttttagtacgtacgcgggagctgggacgaacgctgtcggtgatctattcgccgagtatgtcgtggatctacactgcccgcaaccgtctggcgcattagtccaaacgttgcgaatcactagtgctggggtgcgaggacctgaagttggaccactatactacaacatgacaaaggcagcaactctcattgacctgacgttcttcacaccaggcacatttctgatctcaataggctgcgcagctacttcgtatacttcggagctagtcctgggaggagccacgctgaactcacgaacactcactgccacaggagccgggttttccgggtcctttaacgtcactgtgaccaagcccttagatggcttacgcatacaaggaaccggattcggtgactgtatgacgtttgctgtccgcgcgagggtggccaactctgttactgtctagctgtggctggctggaggataagaagctaacgcgtcgtttcagaatggatgaaaaagcagggtgttcctgatcgggtgaacgatgaggtttttattgcaatgtccaaggcactcaatttcataaatcctgatgagctatctatgcagtgcattttgattgctttgaaccgatttcttcaggagaagcatggttctaaaatggcattcttggatggtaatccgcctgaaaggctatgcatgcctattgttgatcacattcggtctaggggtggagaggtccgcctgaattctcgtattaaaaagatagagctgaatcctgatggaactgtaaaacacttcgcacttagtgatggaactcaaataactggagatgcttatgtttgtgcaacaccagtcgatatcttcaagcttcttgtacctcaagagtggagtgaaattacttatttcaagaaactggagaagttggtgggagttcctgttatcaatgttcatatatggtttgacagaaaactgaacaacacatatgaccaccttcttttcagcaggagttcacttttaagtgtctatgcagacatgtcagtaacctgcaaggaatactatgacccaaaccgttcaatgctggagttggtctttgctcctgcagacgaatggattggtcgaagtgacactgaaatcatcgatgcaactatggaagagctagccaagttatttcctgatgaaagagctcatgtactccacgcttcggcggggctataaggagtacatgatacccccccctatctttcacccagcttgctggggccggccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaa

SEQ ID NO: 18 - DNA, 15854 bp, Artificial Sequence
  Probe
tacgcagttgtcctttggtacattcacaagtttgatcttatcatcaccatcagaagttcagaaagtctcgtagaaaacaaatggaaatgaatactgcttacttagctcaaattcatattccgttgttacaggatacttaaaaaaggtaccaaaggctgttcctaatcatacgctgaagtcgttgccaccaatggcagctgtactgtcatattgtcgtggtttttcaattgctgtacctgatgcaaacgtaatgggtttactaatcttgcacccgccgacttcaaaatgaagagtgctaatttggttcacgtcaccatcaccggttcgaactgtctagaatggcaggcaaagatgattggacaggcatgcagggaaaaagagcaccgatgacgatctatgcgagttcccaccattgcgagcaatgattatcagccacacgacttactcttcagagctaaccactgccatgcagagaaaaagtgaagcatattgtcaggatctacaacgaagtgaaacaatcaggcatgctaaagtgctgaaactttactgatctctcatgttggacaacaaagaatacgggaatacatcagcaacgcaactcttgagctttgcttgctgaatgaccagctagaatttccaagcatttacaggaacatgactttaagtttcagaaaaacaaatacaaggccactaagggcatgttcacttcagcttataagccggctgaaaagctgaaacggctgatttgttgtgagaggaaaacactgtttggtggctgataagccggctgaataagctgaagcgaacaggctgtaaataagcgtggggataacatatcctccagatgacaggcaatctgcaacttgcagcgattcaaatgtacgattaacaaaatatttaagcgctacatgagataatatatcctccaattagggcctttagtattgtcattagctcataagcatggtgcatcctcacatggacgctgcataagaagttcataatagcaacagacatatgaacaaagcatggtgcgcctgcccggccggactagctagtactaccaatcatggaataagctagtaccctaaatgaaattaaaatggtttttagcgattatccacgccgtccagaatactctaatccacaagttgaggccgcccatgaagccgcaaactcagtttatcaccaaagaccaaacatgtggaaatcagtctctattttgtccaagagcatgtggcccttggagctttgcggcttcttcatgttgctacatctcttcaatatgccgatatatttactaagggttgtcaattgttatcttcatcaacttctgatctaatctcaatgtttgctcctcttccggttgagactactgggggatattagaatatgaatagccaaaaagtcttgtatagtctaaaataaagagtctcaaatagttcacttgagcttaggaaccgaatttgtcgtcagcagtgttttttgctcatagtaaattagccaacaatactttctatcacaccttaacagagtactttctttctgccatggcttatcaaccaacagtattttttgtcaaaagcagtgattatctgtcaatcactagcgccccctctgccggtatatctagcgctcccatcggatctgactagagcagatcttgagcgtgggttggtggctcagggcttgcaggaggcgttggccgtcgccggcgtagagcagtagtcgtaggcggatctgcatcttcaagctctcctccggtcgattcgtgtgagtcttcgacctctgctcaggtcgattcatgccggcgaggggctcagtgctcggctcacgacgcgaaattacgagcggcagcagcaaaccgggctttcaagcccggctctcctcgtgagctgccttagggctcgttcgtttaactattgttcccgatggattcattcctgatgataaaaatagtataaatttacacaatgttcctggctggaatcatttcagacctgcattccatgagaaacgaacggggctttagcgggccacgtgacagtgacgaagggtcgcagtcgctgctggacggactacagacagagaggcgaagcatgcaattgaattttcgctagcggaaagttatcatctaatctccaaccctccttcctacggctggatctgaaaattgacgacctgaacccctgaacggtgccggtagcaattgcaggtctcactcacatgctaaatccagcaaccaaacacgaaggaatatatgtgatctggacagaacatgcaagcgaataatacatagagtcgtaccaaccctacacagttcaacgaattaatcactgggttcacgggcatgctcacgtccaaaatcccagcgacattttataagcgctaagcggaatgatccagacggggccagctcgagcaccacatgagtcgtagaggccaattgatgatgtgcctaatagatacatatggtaaggataataatcatttcttactacattattcatacaaaaaataattaagaatcaaaaattatgagaaacacctcttggtgtggtgtagttgtgggtgcatcactccacccattagggtccaaatcttggtgctcacattatgccggggtctcccttacattcttcctatcaattttttttgtaaatctacagtagatgtctataatgaaaattttcaaatatctaaaatagcaacgaaaatctcatatgttacctgtagaagctcaacacttttgtattgcacacaatgttaataaaataaaactcttgctaaaacttgtaatgactacctaataacaacatattgtgttgtatatgaatttaagcccatctaaatattcggaatattcgcttatcattcaaaagatttagatcaacaaaaagaagtgaagaactttatattttggtaggtaaaatgtataacaaaacaaatctttcagaaaatcacttgatatttccaaacacaatacatctaaattgcaataaaaaagaattttagaaaacaaaaacataaaaatatgggtgttgctgtttgaatttcaatactacaaaaggacatatatgtgacgtcatattagtgtcgggcccagcaggaccgccaatgatgtatagcatcagtgttggtcggtgcaaaacccgccactgatatacagctgcgcgtttcccactttcgacctgatgaacatcagtggcgggcgttgcacccgcccgccactaatttttaagtagaggaccttaaatctaagttgacgtatgagaaccattggattaagatataatggcactctcttctcttctacttgctatcgttggattaatatccgacggtcaagcacatcggctcatgtctaacaaaaaaaaggcaacttcttaatagcaaaaccgtaaaaatatatattttattatacaagtctagcccgcgagctgcttggttcaccctgctagttaagatagtaacttgtagctcttcttgttgcgtataagttgttaaacattgtaaaagcctcctcaagtatcatgtatacctgtgatacctcacgacgatttaaacgcacaattgctgtataatggatatagattggttctaggctccagcgatcgattatccatgtaactacgtacaaacgagtaaacctccaaaatcacaccgctgtcacacatcgtctgcacgcagttgcctgaaaccaatccactgcacctagcccacgggttgaataaaaccgcccgcgccggcctcttcaacgtgcatccacgcagtgtgtcattcccgtcacggactctcgtctcatccggccccttctctcgagcaacacccaccaatctcctcgtgggtcgtggcggcctctatataacgccaagacatcgatcagacatccatccatccatccacactcacacagtcgctgtagtagctagcaagcccctaggtgcttgcttgacctactgctctgcccgtgaccagtcgtggatcctcgatatcccggactggcgccaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctgtcgacggatccagaatacctcctggatctaaccaatccgtgagagttggccatggccttggctagaggtgttctctcccagcgcgtcgtgacggcggcagttgacgttacttttggtagtgttgactacagtgacccacgcattgtggcagcactgtgtgatgggggtttgaaggggcgggcgaccgtaaggcgtcaaattgtaactgcgctcaaatggctagtgatggtgctcacttggcccgtaaggatgcccgcgatggcgatcgtgtggtgtctgacatgggtagcactgatggtcactcgaaccaccaggaagatctgctgtgtcgttagcaggttgtactccgagtcctccgccttagtccgtgcatactggcgtgtgtacaataaaaggactagggccgtggcttgcactggcctggtgggttccctggcactgtacggccctgctgctgtgttggtgtgggtgtgtcttctagtggtgttcgtcttttgtacactaccggctgatgcccgatactacatcaaattggccaagaaaatacaggatgcttgggacgcggttgaggaggatgacagcatcaccccagccgctgatggtggaccactggaggttcgctccgggcggaaccggttcgcgtgccgactggcagcgagggcaatcagtcgtgtgggcttgttgaagcccactaaggcaaacgctctcgtgtaccagaaggttatcctcgacgagatgaaagtgctcaacgtccggttcggtgaccgagtacgagtgctgccacttgccgtggtcgcgtgtctggaacggcccgatgctgtggatagggttgagggggtcattgacgccctcacctgtctgcctggcagcctctagggaggccttgtccgccgtgaagggtgcgacaccgacactgaccgcacaaaatttgatctatcagcggttcagggggtgacacgcatggagggaatcacggtacggacagggacctcagccaaaggtgggagaacttggtactcgttcaactcaccggcaacgacatatgagtacattgtccacaactcatcacttaagaacgtagtcaggggacttgtcgagcgggtcttctgtgttgtggacaagaaaactggtgaactggtccggcccccaaaacctgttaaggggctattcaccaagaagctcggtgacgtcggtcaagtagtgagtcaactcgttggttattgcccccactggacacgtcaagaattcttggcgtcttacaatgggccgcgaaaagccagttacgagcgggctgcgctaacgctagacactctgcccttgcgtgaggaggatgcgcatctgagcacctttgtaaaggcggagaagatcaacgtcactctgaaacctgatcctgccccacgagtgattcagccgcgtggacagcggtacaacattgaggtgggaaggtttctgaaacccctggaaccacgcctaatgaaggcgatcgataagctgtgggggtccaccacagctattaaggggtacacggttgagagagtcggggctatcatgaatgagaaagctaacagatttcgtgagcctgtgtttgtgggtttagatgcctctcggtttgaccaacattgttctgccgaggcccttagatgggaacacagtgtttacaacgacatctttcgatctgagtatctcgcaacactcttacagtggcaggtcaacaatagagggactgcctacactaaagagggtactgtgagttacaaggtagaagggtgccgtatgtctggggacatgaacacgtcgatgggaaattatttaatcatgtcctgcttgatctatgccttttgccgggaagttagactgaaagcggaattggctaactgtggtgacgattgcgtgctgtttttggagaaagaggatcttcacaagcttggcactttaccgcagtggtttgtacgtatgggatatacgatgaaggtggaggagccggtgtatgaggtggagcacattgagttctgccaaatgcgccccattcgcacctccagaggatgggtcatggtcaggcgtccggacactgttctaacaaaggattgttgtgttgtcaggggaggaatgactgaggagcggttgaagggatggcttggtagtatgcgcgatggcggtctcagccttgctggggacgtacccatattgggtgccttctaccggtccttcccatcatacgcttctcaggaagcttccgagtacagcgccccacacaagttccgggcgggtaagcagtacggcgctgtcacagacgagagccggtattccttttggctggcgtttgggctcacacccgacgaccagcttgctgtggagagtgaattgtcaaagatggcgtttcatactcgtccggagcaaaaaggaccgtaccagccctcgctacttgactactgcactagaacctgaccagttcaccagccaccttgactactgcactagaacctgaccagttcaccagccatggcgaggaagaagcggagcaaccaggtacagacgggacagggagtgaggcgagcagcaggggctgtcattacagctcctgtagctaggacccgacaagtgagggcccggccacctaaggtcgaggcgttagcgggcggtggttttcgggtcacccatagggagttgatcactaccattgccaactcggctacataccaggcgaacgggggtattgctggattaaagtacaggatgaatccgacgtacggctccaccttgacgtggtgtccggccttggcatccaacttcgaccagtatgtcttccgcaaattgaccttggaatacgtgccgacgtgtgggacaacggagacggggagggtgggcatctggttcgatagggactctgaagatgacccgcctgctgaccgagtggaattggctagtatgggggtacttgtggagactgctccatggagcggtgtcacactacaggtacccacggacaacaccaagagattctgcctcggcgctggtggcaacacggatgccaaactgatagaccttggtcaaatcggttttagtacgtacgcgggagctgggacgaacgctgtcggtgatctattcgccgagtatgtcgtggatctacactgcccgcaaccgtctggcgcattagtccaaacgttgcgaatcactagtgctggggtgcgaggacctgaagttggaccactatactacaacatgacaaaggcagcaactctcattgacctgacgttcttcacaccaggcacatttctgatctcaataggctgcgcagctacttcgtatacttcggagctagtcctgggaggagccacgctgaactcacgaacactcactgccacaggagccgggttttccgggtcctttaacgtcactgtgaccaagcccttagatggcttacgcatacaaggaaccggattcggtgactgtatgacgtttgctgtccgcgcgagggtggccaactctgttactgtctagctgtggctggctggaggataagaagctaaccacttcatgtcgataatcagtcttgacggagagtttgattgtcctccttatcaacccacctcatcccgctttcacttcactcacaaaacgcgcaagtctgctatttgtatcggtccttctactttcggcaaattatggcgagtcccgagggctgggtattacaccccaaccgatgtgacctttgtggttacgccacatatctccgagaaagctggcgttatggcgactgtcaaactcatagacgcatccgacatgagcccatcccgagtgctgttcgagaccaaggcgttcaaccttggccatgggacggtactggaggggtctcaattgccgttttgcctgccaatcggggaatatcctatacacttcgaggtcacggtgtcacgatcacagtttcggggagaacggacaatgtactcaacatcactcgagtggcaaatgatgtgttctcccaccccgttatccagggttcgatctgtgttcgcggttgcgcaccaaccagtgttggatgcggtcccgaatttctcaatgaaaaccaaaaagaagtctagcgtcctgtccggtggtaagggtcaagcgacagaaaagaggattttggctggtggtggtacggcccggggagtggttcccccgggatgcgtagcgccagctgaaggaatcccagtaatcgccactatagaagaccactaggacagcatgtactccacgcttcggcggggctataaggagtacatgatacccccccctatctttcacccagcttgctggggtagcccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatggcgcgtagtgtttttctcagacagttttctaaaaaaagggcgtttctggggaagttcgagatggttcgtaaggtgttactggctcctgtgaaccaatacatgatactgccatgataagggttataattagtcaagcagagtaagaagaaacaacagtagcagtgactccgattcctgaagatgagtcatatttgtcttgtgctcctgctgtatgaaatggatcgcatgtgtatattcgtcgccgcgccgcactggtgtaacctgttgcctcagagtttgcttttagctggttctgttttaaaaataagtactgttttttggttggctgcaagccattctgaacttcagtttaccaattgtttttatgttgtggttgaatattttaattttttatttaatgtttggttctttttttatatatatttgcaaaaatgatacaagtggtcaagttttcatatagtatgggctctatttcctagagctctacctctaggaacgaattttgtggaggttttcttttggctagttaggcaaagtccccatatcttgcaggctaaatcaagaagaagctctgtcaaacagttttttttactgaaaagtgattaaagagtagtttctcctagatcacttcagagtttatcctagagaatcatgggaatcaaattcagttagaggatcatttcttacaaagaatcaactttcgtagagaatctaaagcagaaagagctttgacaaacttacccttagagcaattccaacattctcgcgtgagtttcttcgcgccgttgttttgcggtgacttcatctggacgtcccgcgacatagagacgcttgtattgatcatgagagcttgtgtggtcatacacaatataattgttaaagatgaaagagatgtggaccttaatgagcgattcgactttgatggtgaaaatgtgcaaccttctcatggtatttctactcgcacactagctgaatttattgaagctcataaaaagatccgagacaaagaaatacattttcaattgaaagaagacctaatcaagcacttatgggaattcctaggcttaaggtttaaacagccccctccggcggtgtcccccactgaagaaactatgtgctgtagtatagccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgctgcagaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagagaattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgaccaagcttggcgcgcctagaaggcccggaccgattaaactttaattcggtccgggttaccagagctggtcaccccatacgattggaagcttcaagtttgtacaaaaaagcaggctggccagaatggcccggaccggcggccgcctgcagctctagagaaacttcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctagggatatttctgctagaaagactcttaatcgttctgaacactttcttgaaagttgcggctgaccaccgtacaggaattctctcgcactagtcgggtttgaagcgcgggtgtatctaggagggtaagcctagagcataaattgtaactaccgcgaataaggtcatggccacccagctcacaacgagagctagaagggcaactcgggtttctcgtaagggatcccagcctgcttctaagcaggacgtgaaacaagttgtcaagtccatccttggacaaagcctggaacacaagagagctaacctactcctgcctcccaccgtggttaacactacagggaacatttactgcctgacgcagtttgtgattgagggcgacggcattagccaaaggaccggtcgtgtcattaacttggagcagatggtgttgcgctatcggcgcactctggacaccacatctgcaaactccgggttcctgcgctatatagtgttccttgatactcagaaccaaggcacacttccggcaataacggacgtgctgtcatcccttgacgtatcatctggatacgaggttctgaatgcacagcagaatagatttaagttcctacttgatgaggttgaatcactgtgtgccagtgctaccaacctatccaaggcctccactctgaccttcaatcagaaggtgcaggttcactatgggggcgctgctgatgcggcaacttcaaaccggcgcaatgccgtgttcttcttggagttgtctgacaaggttgccacggggcctcagacgcgcttgggtgtacagctcaagttcactgatgcctagtcattctctgagtgaccgcctacctggttggggtaagacaccaggaacccctctacgaaatgttcagtcggaagctgagaacctcccggtgcatactgacattgtgagggttcggtaggaagttggccaaaggtttccggatataagccacccggttactgtctaactatccccaaattcggccgtgtctgtcgaaagacagctataggatactctggtgaagccaggaaatgttggagcagggatgtttcagcggtccactggctagcccttgcatggttcttgcatggtcctatagcggtgatgtaacggattccatccactctattattagagctacacgccacacccgagctcgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgc

SEQ ID NO: 19 - DNA, 15983 bp, Artificial Sequence
  Probe
tacgcagttgtcctttggtacattcacaagtttgatcttatcatcaccatcagaagttcagaaagtctcgtagaaaacaaatggaaatgaatactgcttacttagctcaaattcatattccgttgttacaggatacttaaaaaaggtaccaaaggctgttcctaatcatacgctgaagtcgttgccaccaatggcagctgtactgtcatattgtcgtggtttttcaattgctgtacctgatgcaaacgtaatgggtttactaatcttgcacccgccgacttcaaaatgaagagtgctaatttggttcacgtcaccatcaccggttcgaactgtctagaatggcaggcaaagatgattggacaggcatgcagggaaaaagagcaccgatgacgatctatgcgagttcccaccattgcgagcaatgattatcagccacacgacttactcttcagagctaaccactgccatgcagagaaaaagtgaagcatattgtcaggatctacaacgaagtgaaacaatcaggcatgctaaagtgctgaaactttactgatctctcatgttggacaacaaagaatacgggaatacatcagcaacgcaactcttgagctttgcttgctgaatgaccagctagaatttccaagcatttacaggaacatgactttaagtttcagaaaaacaaatacaaggccactaagggcatgttcacttcagcttataagccggctgaaaagctgaaacggctgatttgttgtgagaggaaaacactgtttggtggctgataagccggctgaataagctgaagcgaacaggctgtaaataagcgtggggataacatatcctccagatgacaggcaatctgcaacttgcagcgattcaaatgtacgattaacaaaatatttaagcgctacatgagataatatatcctccaattagggcctttagtattgtcattagctcataagcatggtgcatcctcacatggacgctgcataagaagttcataatagcaacagacatatgaacaaagcatggtgcgcctgcccggccggactagctagtactaccaatcatggaataagctagtaccctaaatgaaattaaaatggtttttagcgattatccacgccgtccagaatactctaatccacaagttgaggccgcccatgaagccgcaaactcagtttatcaccaaagaccaaacatgtggaaatcagtctctattttgtccaagagcatgtggcccttggagctttgcggcttcttcatgttgctacatctcttcaatatgccgatatatttactaagggttgtcaattgttatcttcatcaacttctgatctaatctcaatgtttgctcctcttccggttgagactactgggggatattagaatatgaatagccaaaaagtcttgtatagtctaaaataaagagtctcaaatagttcacttgagcttaggaaccgaatttgtcgtcagcagtgttttttgctcatagtaaattagccaacaatactttctatcacaccttaacagagtactttctttctgccatggcttatcaaccaacagtattttttgtcaaaagcagtgattatctgtcaatcactagcgccccctctgccggtatatctagcgctcccatcggatctgactagagcagatcttgagcgtgggttggtggctcagggcttgcaggaggcgttggccgtcgccggcgtagagcagtagtcgtaggcggatctgcatcttcaagctctcctccggtcgattcgtgtgagtcttcgacctctgctcaggtcgattcatgccggcgaggggctcagtgctcggctcacgacgcgaaattacgagcggcagcagcaaaccgggctttcaagcccggctctcctcgtgagctgccttagggctcgttcgtttaactattgttcccgatggattcattcctgatgataaaaatagtataaatttacacaatgttcctggctggaatcatttcagacctgcattccatgagaaacgaacggggctttagcgggccacgtgacagtgacgaagggtcgcagtcgctgctggacggactacagacagagaggcgaagcatgcaattgaattttcgctagcggaaagttatcatctaatctccaaccctccttcctacggctggatctgaaaattgacgacctgaacccctgaacggtgccggtagcaattgcaggtctcactcacatgctaaatccagcaaccaaacacgaaggaatatatgtgatctggacagaacatgcaagcgaataatacatagagtcgtaccaaccctacacagttcaacgaattaatcactgggttcacgggcatgctcacgtccaaaatcccagcgacattttataagcgctaagcggaatgatccagacggggccagctcgagcaccacatgagtcgtagaggccaattgatgatgtgcctaatagatacatatggtaaggataataatcatttcttactacattattcatacaaaaaataattaagaatcaaaaattatgagaaacacctcttggtgtggtgtagttgtgggtgcatcactccacccattagggtccaaatcttggtgctcacattatgccggggtctcccttacattcttcctatcaattttttttgtaaatctacagtagatgtctataatgaaaattttcaaatatctaaaatagcaacgaaaatctcatatgttacctgtagaagctcaacacttttgtattgcacacaatgttaataaaataaaactcttgctaaaacttgtaatgactacctaataacaacatattgtgttgtatatgaatttaagcccatctaaatattcggaatattcgcttatcattcaaaagatttagatcaacaaaaagaagtgaagaactttatattttggtaggtaaaatgtataacaaaacaaatctttcagaaaatcacttgatatttccaaacacaatacatctaaattgcaataaaaaagaattttagaaaacaaaaacataaaaatatgggtgttgctgtttgaatttcaatactacaaaaggacatatatgtgacgtcatattagtgtcgggcccagcaggaccgccaatgatgtatagcatcagtgttggtcggtgcaaaacccgccactgatatacagctgcgcgtttcccactttcgacctgatgaacatcagtggcgggcgttgcacccgcccgccactaatttttaagtagaggaccttaaatctaagttgacgtatgagaaccattggattaagatataatggcactctcttctcttctacttgctatcgttggattaatatccgacggtcaagcacatcggctcatgtctaacaaaaaaaaggcaacttcttaatagcaaaaccgtaaaaatatatattttattatacaagtctagcccgcgagctgcttggttcaccctgctagttaagatagtaacttgtagctcttcttgttgcgtataagttgttaaacattgtaaaagcctcctcaagtatcatgtatacctgtgatacctcacgacgatttaaacgcacaattgctgtataatggatatagattggttctaggctccagcgatcgattatccatgtaactacgtacaaacgagtaaacctccaaaatcacaccgctgtcacacatcgtctgcacgcagttgcctgaaaccaatccactgcacctagcccacgggttgaataaaaccgcccgcgccggcctcttcaacgtgcatccacgcagtgtgtcattcccgtcacggactctcgtctcatccggccccttctctcgagcaacacccaccaatctcctcgtgggtcgtggcggcctctatataacgccaagacatcgatcagacatccatccatccatccacactcacacagtcgctgtagtagctagcaagcccctaggtgcttgcttgacctactgctctgcccgtgaccagtcgtggatcctcgatatcccggactggcgccaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctgtcgacggatccagaatacctcctggatctaaccaatccgtgagagttggccatggccttggctagaggtgttctctcccagcgcgtcgtgacggcggcagttgacgttacttttggtagtgttgactacagtgacccacgcattgtggcagcactgtgtgatgggggtttgaaggggcgggcgaccgtaaggcgtcaaattgtaactgcgctcaaatggctagtgatggtgctcacttggcccgtaaggatgcccgcgatggcgatcgtgtggtgtctgacatgggtagcactgatggtcactcgaaccaccaggaagatctgctgtgtcgttagcaggttgtactccgagtcctccgccttagtccgtgcatactggcgtgtgtacaataaaaggactagggccgtggcttgcactggcctggtgggttccctggcactgtacggccctgctgctgtgttggtgtgggtgtgtcttctagtggtgttcgtcttttgtacactaccggctgatgcccgatactacatcaaattggccaagaaaatacaggatgcttgggacgcggttgaggaggatgacagcatcaccccagccgctgatggtggaccactggaggttcgctccgggcggaaccggttcgcgtgccgactggcagcgagggcaatcagtcgtgtgggcttgttgaagcccactaaggcaaacgctctcgtgtaccagaaggttatcctcgacgagatgaaagtgctcaacgtccggttcggtgaccgagtacgagtgctgccacttgccgtggtcgcgtgtctggaacggcccgatgctgtggatagggttgagggggtcattgacgccctcacctgtctgcctggcagcctctagggaggccttgtccgccgtgaagggtgcgacaccgacactgaccgcacaaaatttgatctatcagcggttcagggggtgacacgcatggagggaatcacggtacggacagggacctcagccaaaggtgggagaacttggtactcgttcaactcaccggcaacgacatatgagtacattgtccacaactcatcacttaagaacgtagtcaggggacttgtcgagcgggtcttctgtgttgtggacaagaaaactggtgaactggtccggcccccaaaacctgttaaggggctattcaccaagaagctcggtgacgtcggtcaagtagtgagtcaactcgttggttattgcccccactggacacgtcaagaattcttggcgtcttacaatgggccgcgaaaagccagttacgagcgggctgcgctaacgctagacactctgcccttgcgtgaggaggatgcgcatctgagcacctttgtaaaggcggagaagatcaacgtcactctgaaacctgatcctgccccacgagtgattcagccgcgtggacagcggtacaacattgaggtgggaaggtttctgaaacccctggaaccacgcctaatgaaggcgatcgataagctgtgggggtccaccacagctattaaggggtacacggttgagagagtcggggctatcatgaatgagaaagctaacagatttcgtgagcctgtgtttgtgggtttagatgcctctcggtttgaccaacattgttctgccgaggcccttagatgggaacacagtgtttacaacgacatctttcgatctgagtatctcgcaacactcttacagtggcaggtcaacaatagagggactgcctacactaaagagggtactgtgagttacaaggtagaagggtgccgtatgtctggggacatgaacacgtcgatgggaaattatttaatcatgtcctgcttgatctatgccttttgccgggaagttagactgaaagcggaattggctaactgtggtgacgattgcgtgctgtttttggagaaagaggatcttcacaagcttggcactttaccgcagtggtttgtacgtatgggatatacgatgaaggtggaggagccggtgtatgaggtggagcacattgagttctgccaaatgcgccccattcgcacctccagaggatgggtcatggtcaggcgtccggacactgttctaacaaaggattgttgtgttgtcaggggaggaatgactgaggagcggttgaagggatggcttggtagtatgcgcgatggcggtctcagccttgctggggacgtacccatattgggtgccttctaccggtccttcccatcatacgcttctcaggaagcttccgagtacagcgccccacacaagttccgggcgggtaagcagtacggcgctgtcacagacgagagccggtattccttttggctggcgtttgggctcacacccgacgaccagcttgctgtggagagtgaattgtcaaagatggcgtttcatactcgtccggagcaaaaaggaccgtaccagccctcgctacttgactactgcactagaacctgaccagttcaccagccaccttgactactgcactagaacctgaccagttcaccagccatggcgaggaagaagcggagcaaccaggtacagacgggacagggagtgaggcgagcagcaggggctgtcattacagctcctgtagctaggacccgacaagtgagggcccggccacctaaggtcgaggcgttagcgggcggtggttttcgggtcacccatagggagttgatcactaccattgccaactcggctacataccaggcgaacgggggtattgctggattaaagtacaggatgaatccgacgtacggctccaccttgacgtggtgtccggccttggcatccaacttcgaccagtatgtcttccgcaaattgaccttggaatacgtgccgacgtgtgggacaacggagacggggagggtgggcatctggttcgatagggactctgaagatgacccgcctgctgaccgagtggaattggctagtatgggggtacttgtggagactgctccatggagcggtgtcacactacaggtacccacggacaacaccaagagattctgcctcggcgctggtggcaacacggatgccaaactgatagaccttggtcaaatcggttttagtacgtacgcgggagctgggacgaacgctgtcggtgatctattcgccgagtatgtcgtggatctacactgcccgcaaccgtctggcgcattagtccaaacgttgcgaatcactagtgctggggtgcgaggacctgaagttggaccactatactacaacatgacaaaggcagcaactctcattgacctgacgttcttcacaccaggcacatttctgatctcaataggctgcgcagctacttcgtatacttcggagctagtcctgggaggagccacgctgaactcacgaacactcactgccacaggagccgggttttccgggtcctttaacgtcactgtgaccaagcccttagatggcttacgcatacaaggaaccggattcggtgactgtatgacgtttgctgtccgcgcgagggtggccaactctgttactgtctagctgtggctggctggaggataagaagctaaccacttcatgtcgataatcagtcttgacggagagtttgattgtcctccttatcaacccacctcatcccgctttcacttcactcacaaaacgcgcaagtctgctatttgtatcggtccttctactttcggcaaattatggcgagtcccgagggctgggtattacaccccaaccgatgtgacctttgtggttacgccacatatctccgagaaagctggcgttatggcgactgtcaaactcatagacgcatccgacatgagcccatcccgagtgctgttcgagaccaaggcgttcaaccttggccatgggacggtactggaggggtctcaattgccgttttgcctgccaatcggggaatatcctatacacttcgaggtcacggtgtcacgatcacagtttcggggagaacggacaatgtactcaacatcactcgagtggcaaatgatgtgttctcccaccccgttatccagggttcgatctgtgttcgcggttgcgcaccaaccagtgttggatgcggtcccgaatttctcaatgaaaaccaaaaagaagtctagcgtcctgtccggtggtaagggtcaagcgacagaaaagaggattttggctggtggtggtacggcccggggagtggttcccccgggatgcgtagcgccagctgaaggaatcccagtaatcgccactatagaagaccactaggacagcatgtactccacgcttcggcggggctataaggagtacatgatacccccccctatctttcacccagcttgctggggtagcccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatggcgcgtagtgtttttctcagacagttttctaaaaaaagggcgtttctggggaagttcgagatggttcgtaaggtgttactggctcctgtgaaccaatacatgatactgccatgataagggttataattagtcaagcagagtaagaagaaacaacagtagcagtgactccgattcctgaagatgagtcatatttgtcttgtgctcctgctgtatgaaatggatcgcatgtgtatattcgtcgccgcgccgcactggtgtaacctgttgcctcagagtttgcttttagctggttctgttttaaaaataagtactgttttttggttggctgcaagccattctgaacttcagtttaccaattgtttttatgttgtggttgaatattttaattttttatttaatgtttggttctttttttatatatatttgcaaaaatgatacaagtggtcaagttttcatatagtatgggctctatttcctagagctctacctctaggaacgaattttgtggaggttttcttttggctagttaggcaaagtccccatatcttgcaggctaaatcaagaagaagctctgtcaaacagttttttttactgaaaagtgattaaagagtagtttctcctagatcacttcagagtttatcctagagaatcatgggaatcaaattcagttagaggatcatttcttacaaagaatcaactttcgtagagaatctaaagcagaaagagctttgacaaacttacccttagagcaattccaacattctcgcgtgagtttcttcgcgccgttgttttgcggtgacttcatctggacgtcccgcgacatagagacgcttgtattgatcatgagagcttgtgtggtcatacacaatataattgttaaagatgaaagagatgtggaccttaatgagcgattcgactttgatggtgaaaatgtgcaaccttctcatggtatttctactcgcacactagctgaatttattgaagctcataaaaagatccgagacaaagaaatacattttcaattgaaagaagacctaatcaagcacttatgggaattcctaggcttaaggtttaaacagccccctccggcggtgtcccccactgaagaaactatgtgctgtagtatagccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgctgcagaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagagaattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgaccaagcttggcgcgcctagaaggcccggaccgattaaactttaattcggtccgggttaccagagctggtcaccccatacgattggaagcttcaagtttgtacaaaaaagcaggctggccagaatggcccggaccggcggccgccctgcagctctagagaaacttcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctagggatatttctgctagaaagactcttaatcgttctgaacactttcttgaaagttgcggctgaccaccgtacaggaattctctcgcactagtcgggtttgaagcgcgggtgtatctaggagggtaagcctagagcataaattgtaactaccgcgaataaggtcatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctccggactcagatctcgatagtcattctctgagtgaccgcctacctggttggggtaagacaccaggaacccctctacgaaatgttcagtcggaagctgagaacctcccggtgcatactgacattgtgagggttcggtaggaagttggccaaaggtttccggatataagccacccggttactgtctaactatccccaaattcggccgtgtctgtcgaaagacagctataggatactctggtgaagccaggaaatgttggagcagggatgtttcagcggtccactggctagcccttgcatggttcttgcatggtcctatagcggtgatgtaacggattccatccactctattattagagctacacgccacacccgagctcgataccctgtcaccggatgtgctttccggtctgatgagtccgtgaggacgaaacaggactgtcaggtggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaa

SEQ ID NO: 20 - DNA, 11547 bp, Artificial Sequence
  Probe
gccagaagatagaagatatcctggacctgcaagatgtcagcaatgacgattgaaagattcccaggatagccggcggacgtggtggacccagtctaggtgcgatgcttagtcacgcacgatgactctgtcggaaggcatctttactttcggcaaactttaataatactttaggaaaagtattgtacaagttaggtgcagaatcaataatgcacccagctttagtcttgtctactgaattattgtgtcggttgcattattggatgcctgcgtgcaccctaagcaatccccggctctcatctctataagaggagcctttgtattcagttgcaagcatgcaagtcacacactgcaagcttacttctgagcaaaaagagttttgagtgaaataaatttgaagttcccccttacatcttgctcgagaccggtgatcttgtaaggttcccttccctcctcccctcacacccctgttcgtgttccttcggatcggatctcagtggtgatgttagacgtccgcggctgcctacgtagtggcattgccgcccgaaaggtttgtttaggtggggtagatccgaaacaggccggatctggaccatgtccgcggcggggcggcgggacttgatcgcgtagctgtcgtgtgcatttctccctaccagtggcggaatcggcgatgtggacctaagggctaaggcttatctgctgccttgaccatttcgtcgctgacaaaaacaaagtgacaatcatgccgttctctgtttgtttatctggatcgttattacgctgtgaatcctgcgatatgtggctaagtgatttttcttctttttctgggggcagtttagcctttgacccagtcctaggtgtggtcactaggactgtgtagcatgatgagtgaggttgcagcaggctgattgctagtggacgtttttttccccaatttgttaggttttcacgctccaggttgtgcaagtaattttgctagtgattgtgtgatccatcttcaacgttgaaccttgtttttccccctaaaacccccaacaggaaatcttgccccgacttctattgcaaaaattgtaacgcttagcaccctgattgactcaattcctgtcactaggcatgctcggtcaaaagcagatgatttaccacttagaaactgccctgcccctgctttccacatagcatttcgaactttttgactactattgacacccccctaacttgccgaactatttctctcttcagctactatttacctagttataattacataaatgtttgtgtgtatcttgtgcagggatccagaatacctcctggatctaaccaatccgtgagagttggccatggccttggctagaggtgttctctcccagcgcgtcgtgacggcggcagttgacgttacttttggtagtgttgactacagtgacccacgcattgtggcagcactgtgtgatgggggtttgaaggggcgggcgaccgtaaggcgtcaaattgtaactgcgctcaaatggctagtgatggtgctcacttggcccgtaaggatgcccgcgatggcgatcgtgtggtgtctgacatgggtagcactgatggtcactcgaaccaccaggaagatctgctgtgtcgttagcaggttgtactccgagtcctccgccttagtccgtgcatactggcgtgtgtacaataaaaggactagggccgtggcttgcactggcctggtgggttccctggcactgtacggccctgctgctgtgttggtgtgggtgtgtcttctagtggtgttcgtcttttgtacactaccggctgatgcccgatactacatcaaattggccaagaaaatacaggatgcttgggacgcggttgaggaggatgacagcatcaccccagccgctgatggtggaccactggaggttcgctccgggcggaaccggttcgcgtgccgactggcagcgagggcaatcagtcgtgtgggcttgttgaagcccactaaggcaaacgctctcgtgtaccagaaggttatcctcgacgagatgaaagtgctcaacgtccggttcggtgaccgagtacgagtgctgccacttgccgtggtcgcgtgtctggaacggcccgatgctgtggatagggttgagggggtcattgacgccctcacctgtctgcctggcagcctctagggaggccttgtccgccgtgaagggtgcgacaccgacactgaccgcacaaaatttgatctatcagcggttcagggggtgacacgcatggagggaatcacggtacggacagggacctcagccaaaggtgggagaacttggtactcgttcaactcaccggcaacgacatatgagtacattgtccacaactcatcacttaagaacgtagtcaggggacttgtcgagcgggtcttctgtgttgtggacaagaaaactggtgaactggtccggcccccaaaacctgttaaggggctattcaccaagaagctcggtgacgtcggtcaagtagtgagtcaactcgttggttattgcccccactggacacgtcaagaattcttggcgtcttacaatgggccgcgaaaagccagttacgagcgggctgcgctaacgctagacactctgcccttgcgtgaggaggatgcgcatctgagcacctttgtaaaggcggagaagatcaacgtcactctgaaacctgatcctgccccacgagtgattcagccgcgtggacagcggtacaacattgaggtgggaaggtttctgaaacccctggaaccacgcctaatgaaggcgatcgataagctgtgggggtccaccacagctattaaggggtacacggttgagagagtcggggctatcatgaatgagaaagctaacagatttcgtgagcctgtgtttgtgggtttagatgcctctcggtttgaccaacattgttctgccgaggcccttagatgggaacacagtgtttacaacgacatctttcgatctgagtatctcgcaacactcttacagtggcaggtcaacaatagagggactgcctacactaaagagggtactgtgagttacaaggtagaagggtgccgtatgtctggggacatgaacacgtcgatgggaaattatttaatcatgtcctgcttgatctatgccttttgccgggaagttagactgaaagcggaattggctaactgtggtgacgattgcgtgctgtttttggagaaagaggatcttcacaagcttggcactttaccgcagtggtttgtacgtatgggatatacgatgaaggtggaggagccggtgtatgaggtggagcacattgagttctgccaaatgcgccccattcgcacctccagaggatgggtcatggtcaggcgtccggacactgttctaacaaaggattgttgtgttgtcaggggaggaatgactgaggagcggttgaagggatggcttggtagtatgcgcgatggcggtctcagccttgctggggacgtacccatattgggtgccttctaccggtccttcccatcatacgcttctcaggaagcttccgagtacagcgccccacacaagttccgggcgggtaagcagtacggcgctgtcacagacgagagccggtattccttttggctggcgtttgggctcacacccgacgaccagcttgctgtggagagtgaattgtcaaagatggcgtttcatactcgtccggagcaaaaaggaccgtaccagccctcgctacttgactactgcactagaacctgaccagttcaccagccaccttgactactgcactagaacctgaccagttcaccagccatggcgaggaagaagcggagcaaccaggtatttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattatcgatgggccccggccgcctgcagctctagagaaacttcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctagggatatttctgctagaaagactcttaatcgttctgaacactttcttgaaagttgcggctgaccaccgtacaggaattctctcgcactagtcgggtttgaagcgcgggtgtatctaggagggtaagcctagagcataaattgtaactaccgcgaataaggtcatggccacccagctcacaacgagagctagaagggcaactcgggtttctcgtaagggatcccagcctgcttctaagcaggacgtgaaacaagttgtcaagtccatccttggacaaagcctggaacacaagagagctaacctactcctgcctcccaccgtggttaacactacagggaacatttactgcctgacgcagtttgtgattgagggcgacggcattagccaaaggaccggtcgtgtcattaacttggagcagatggtgttgcgctatcggcgcactctggacaccacatctgcaaactccgggttcctgcgctatatagtgttccttgatactcagaaccaaggcacacttccggcaataacggacgtgctgtcatcccttgacgtatcatctggatacgaggttctgaatgcacagcagaatagatttaagttcctacttgatgaggttgaatcactgtgtgccagtgctaccaacctatccaaggcctccactctgaccttcaatcagaaggtgcaggttcactatgggggcgctgctgatgcggcaacttcaaaccggcgcaatgccgtgttcttcttggagttgtctgacaaggttgccacggggcctcagacgcgcttgggtgtacagctcaagttcactgatgcctagtcattctctgagtgaccgcctacctggttggggtaagacaccaggaacccctctacgaaatgttcagtcggaagctgagaacctcccggtgcatactgacattgtgagggttcggtaggaagttggccaaaggtttccggatataagccacccggttactgtctaactatccccaaattcggccgtgtctgtcgaaagacagctataggatactctggtgaagccaggaaatgttggagcagggatgtttcagcggtccactggctagcccttgcatggttcttgcatggtcctatagcggtgatgtaacggattccatccactctattattagagctacacgccacacccgagctcgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattatcgatgggccccggccgaagctggccgcgggcatgtggtacctaagggcccataggcgcgcccggtgcatgcaagcttgcttcaagggcccgtttgtatcaagtttgtacaaaaaagcaggctggccagaatggcccggaccggcggccgccctgcagctctagagaaacttcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctagggatatttctgctagaaagactcttaatcgttctgaacactttcttgaaagttgcggctgaccaccgtacaggaattctctcgcactagtcgggtttgaagcgcgggtgtatctaggagggtaagcctagagcataaattgtaactaccgcgaataaggtcatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctccggactcagatctcgatagtcattctctgagtgaccgcctacctggttggggtaagacaccaggaacccctctacgaaatgttcagtcggaagctgagaacctcccggtgcatactgacattgtgagggttcggtaggaagttggccaaaggtttccggatataagccacccggttactgtctaactatccccaaattcggccgtgtctgtcgaaagacagctataggatactctggtgaagccaggaaatgttggagcagggatgtttcagcggtccactggctagcccttgcatggttcttgcatggtcctatagcggtgatgtaacggattccatccactctattattagagctacacgccacacccgagctcgataccctgtcaccggatgtgctttccggtctgatgagtccgtgaggacgaaacaggactgtcaggtggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgc

SEQ ID NO: 21 - DNA, 8984 bp, Artificial Sequence
  Probe
gccagaagatagaagatatcctggacctgcaagatgtcagcaatgacgattgaaagattcccaggatagccggcggacgtggtggacccagtctaggtgcgatgcttagtcacgcacgatgactctgtcggaaggcatctttactttcggcaaactttaataatactttaggaaaagtattgtacaagttaggtgcagaatcaataatgcacccagctttagtcttgtctactgaattattgtgtcggttgcattattggatgcctgcgtgcaccctaagcaatccccggctctcatctctataagaggagcctttgtattcagttgcaagcatgcaagtcacacactgcaagcttacttctgagcaaaaagagttttgagtgaaataaatttgaagttcccccttacatcttgctcgagaccggtgatcttgtaaggttcccttccctcctcccctcacacccctgttcgtgttccttcggatcggatctcagtggtgatgttagacgtccgcggctgcctacgtagtggcattgccgcccgaaaggtttgtttaggtggggtagatccgaaacaggccggatctggaccatgtccgcggcggggcggcgggacttgatcgcgtagctgtcgtgtgcatttctccctaccagtggcggaatcggcgatgtggacctaagggctaaggcttatctgctgccttgaccatttcgtcgctgacaaaaacaaagtgacaatcatgccgttctctgtttgtttatctggatcgttattacgctgtgaatcctgcgatatgtggctaagtgatttttcttctttttctgggggcagtttagcctttgacccagtcctaggtgtggtcactaggactgtgtagcatgatgagtgaggttgcagcaggctgattgctagtggacgtttttttccccaatttgttaggttttcacgctccaggttgtgcaagtaattttgctagtgattgtgtgatccatcttcaacgttgaaccttgtttttccccctaaaacccccaacaggaaatcttgccccgacttctattgcaaaaattgtaacgcttagcaccctgattgactcaattcctgtcactaggcatgctcggtcaaaagcagatgatttaccacttagaaactgccctgcccctgctttccacatagcatttcgaactttttgactactattgacacccccctaacttgccgaactatttctctcttcagctactatttacctagttataattacataaatgtttgtgtgtatcttgtgcagggatccagaatacctcctggatctaaccaatccgtgagagttggccatggccttggctagaggtgttctctcccagcgcgtcgtgacggcggcagttgacgttacttttggtagtgttgactacagtgacccacgcattgtggcagcactgtgtgatgggggtttgaaggggcgggcgaccgtaaggcgtcaaattgtaactgcgctcaaatggctagtgatggtgctcacttggcccgtaaggatgcccgcgatggcgatcgtgtggtgtctgacatgggtagcactgatggtcactcgaaccaccaggaagatctgctgtgtcgttagcaggttgtactccgagtcctccgccttagtccgtgcatactggcgtgtgtacaataaaaggactagggccgtggcttgcactggcctggtgggttccctggcactgtacggccctgctgctgtgttggtgtgggtgtgtcttctagtggtgttcgtcttttgtacactaccggctgatgcccgatactacatcaaattggccaagaaaatacaggatgcttgggacgcggttgaggaggatgacagcatcaccccagccgctgatggtggaccactggaggttcgctccgggcggaaccggttcgcgtgccgactggcagcgagggcaatcagtcgtgtgggcttgttgaagcccactaaggcaaacgctctcgtgtaccagaaggttatcctcgacgagatgaaagtgctcaacgtccggttcggtgaccgagtacgagtgctgccacttgccgtggtcgcgtgtctggaacggcccgatgctgtggatagggttgagggggtcattgacgccctcacctgtctgcctggcagcctctagggaggccttgtccgccgtgaagggtgcgacaccgacactgaccgcacaaaatttgatctatcagcggttcagggggtgacacgcatggagggaatcacggtacggacagggacctcagccaaaggtgggagaacttggtactcgttcaactcaccggcaacgacatatgagtacattgtccacaactcatcacttaagaacgtagtcaggggacttgtcgagcgggtcttctgtgttgtggacaagaaaactggtgaactggtccggcccccaaaacctgttaaggggctattcaccaagaagctcggtgacgtcggtcaagtagtgagtcaactcgttggttattgcccccactggacacgtcaagaattcttggcgtcttacaatgggccgcgaaaagccagttacgagcgggctgcgctaacgctagacactctgcccttgcgtgaggaggatgcgcatctgagcacctttgtaaaggcggagaagatcaacgtcactctgaaacctgatcctgccccacgagtgattcagccgcgtggacagcggtacaacattgaggtgggaaggtttctgaaacccctggaaccacgcctaatgaaggcgatcgataagctgtgggggtccaccacagctattaaggggtacacggttgagagagtcggggctatcatgaatgagaaagctaacagatttcgtgagcctgtgtttgtgggtttagatgcctctcggtttgaccaacattgttctgccgaggcccttagatgggaacacagtgtttacaacgacatctttcgatctgagtatctcgcaacactcttacagtggcaggtcaacaatagagggactgcctacactaaagagggtactgtgagttacaaggtagaagggtgccgtatgtctggggacatgaacacgtcgatgggaaattatttaatcatgtcctgcttgatctatgccttttgccgggaagttagactgaaagcggaattggctaactgtggtgacgattgcgtgctgtttttggagaaagaggatcttcacaagcttggcactttaccgcagtggtttgtacgtatgggatatacgatgaaggtggaggagccggtgtatgaggtggagcacattgagttctgccaaatgcgccccattcgcacctccagaggatgggtcatggtcaggcgtccggacactgttctaacaaaggattgttgtgttgtcaggggaggaatgactgaggagcggttgaagggatggcttggtagtatgcgcgatggcggtctcagccttgctggggacgtacccatattgggtgccttctaccggtccttcccatcatacgcttctcaggaagcttccgagtacagcgccccacacaagttccgggcgggtaagcagtacggcgctgtcacagacgagagccggtattccttttggctggcgtttgggctcacacccgacgaccagcttgctgtggagagtgaattgtcaaagatggcgtttcatactcgtccggagcaaaaaggaccgtaccagccctcgctacttgactactgcactagaacctgaccagttcaccagccaccttgactactgcactagaacctgaccagttcaccagccatggcgaggaagaagcggagcaaccaggtatttaaatcctacgaatacgccggaagccacaatttctgcatcgggatttttttcttcgtttagattccaggttcctcctactcccaagaagtagccttgatatccggttcggtacaagatgagacatatcaaattgatcaccaacttcagcaatttcaggacaatggtccccacagtctcgatacttgtcattttcgtaaaaaatcgaacttattatctgcgctatatagtgttccttgatactcagaaccaaggcacacttccggcaataacggacgtgctgtcatcccttgacgtatcatctggatacgaggttcgaacataataagttcgattttttacgaaaatgacaagtatcgagactgtggggaccattgtcctgaaattgctgaagttggtgatcaatttgatatgtctcatcttgtaccgaaccggatatcaaggctacttcttgggagtaggaggaacctggaatctaaacgaagaaaaaaatcccgatgcagaaattgtggcttccggcgtattcgtaggacgcgttcatgtcgataatcagtcttgacggagagtttgattgtcctccttatcaacccacctcatcccgctttcacttcactcacaaaacgcgcaagtctgctatttgtatcggtccttctactttcggcaaattatggcgagtcccgagggctgggtattacaccccaaccgatgtgacctttgtggttacgccacatatctccgagaaagctggcgttatggcgactgtcaaactcatagacgcatccgacatgagcccatcccgagtgctgttcgagaccaaggcgttcaaccttggccatgggacggtactggaggggtctcaattgccgttttgcctgccaatcggggaatatcctatacacttcgaggtcacggtgtcacgatcacagtttcggggagaacggacaatgtactcaacatcactcgagtggcaaatgatgtgttctcccaccccgttatccagggttcgatctgtgttcgcggttgcgcaccaaccagtgttggatgcggtcccgaatttctcaatgaaaaccaaaaagaagtctagcgtcctgtccggtggtaagggtcaagcgacagaaaagaggattttggctggtggtggtacggcccggggagtggttcccccgggatgcgtagcgccagctgaaggaatcccagtaatcgccactatagaagaccactaggagctcatgtactccacgcttcggcggggctataaggagtacatgatacccccccctatctttcacccagcttgctggggccggccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattatcgatgggccccggccgaagctggccgcgggcatgtggtacctaagggcccataggcgcgcccggtgcatgcaagcttgcttcaagggcccgtttgtatcaagtttgtacaaaaaagcaggctggccagaatggcccggaccggcggccgcctgcagctctagagaaacttcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatggcgaggaagaagcggagcaaccaggtacagacgggacagggagtgaggcgagcagcaggggctgtcattacagctcctgtagctaggacccgacaagtgagggcccggccacctaaggtcgaggcgttagcgggcggtggttttcgggtcacccatagggagttgatcactaccattgccaactcggctacataccaggcgaacgggggtattgctggattaaagtacaggatgaatccgacgtacggctccaccttgacgtggtgtccggccttggcatccaacttcgaccagtatgtcttccgcaaattgaccttggaatacgtgccgacgtgtgggacaacggagacggggagggtgggcatctggttcgatagggactctgaagatgacccgcctgctgaccgagtggaattggctagtatgggggtacttgtggagactgctccatggagcggtgtcacactacaggtacccacggacaacaccaagagattctgcctcggcgctggtggcaacacggatgccaaactgatagaccttggtcaaatcggttttagtacgtacgcgggagctgggacgaacgctgtcggtgatctattcgccgagtatgtcgtggatctacactgcccgcaaccgtctggcgcattagtccaaacgttgcgaatcactagtgctggggtgcgaggacctgaagttggaccactatactacaacatgacaaaggcagcaactctcattgacctgacgttcttcacaccaggcacatttctgatctcaataggctgcgcagctacttcgtatacttcggagctagtcctgggaggagccacgctgaactcacgaacactcactgccacaggagccgggttttccgggtcctttaacgtcactgtgaccaagcccttagatggcttacgcatacaaggaaccggattcggtgactgtatgacgtttgctgtccgcgcgagggtggccaactctgttactgtctaggagctcgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaa

SEQ ID NO: 22 - DNA, 10327 bp, Artificial Sequence
  Probe
atcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagcaacgagatcatgagccaatcaaagaggagtgatgtagacctaaagcaataatggagccatgacgtaagggcttacgcccatacgaaataattaaaggctgatgtgacctgtcggtctctcagaacctttactttttatgtttggcgtgtatttttaaatttccacggcaatgacgatgtgaccgtcgacccactaaaacattgctttgtcaaaagctaaaaaagatgatgcccgacagccacttgtgtgaagcatgagaagccggtccctccactaagaaaattagtgaagcatcttccagtggtccctccactcacagctcaatcagtgagcaacaggacgaaggaaatgacgtaagccatgacgtctaatcccattcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccagaatacctcctggatctaaccaatccgtgagagttggccatggccttggctagaggtgttctctcccagcgcgtcgtgacggcggcagttgacgttacttttggtagtgttgactacagtgacccacgcattgtggcagcactgtgtgatgggggtttgaaggggcgggcgaccgtaaggcgtcaaattgtaactgcgctcaaatggctagtgatggtgctcacttggcccgtaaggatgcccgcgatggcgatcgtgtggtgtctgacatgggtagcactgatggtcactcgaaccaccaggaagatctgctgtgtcgttagcaggttgtactccgagtcctccgccttagtccgtgcatactggcgtgtgtacaataaaaggactagggccgtggcttgcactggcctggtgggttccctggcactgtacggccctgctgctgtgttggtgtgggtgtgtcttctagtggtgttcgtcttttgtacactaccggctgatgcccgatactacatcaaattggccaagaaaatacaggatgcttgggacgcggttgaggaggatgacagcatcaccccagccgctgatggtggaccactggaggttcgctccgggcggaaccggttcgcgtgccgactggcagcgagggcaatcagtcgtgtgggcttgttgaagcccactaaggcaaacgctctcgtgtaccagaaggttatcctcgacgagatgaaagtgctcaacgtccggttcggtgaccgagtacgagtgctgccacttgccgtggtcgcgtgtctggaacggcccgatgctgtggatagggttgagggggtcattgacgccctcacctgtctgcctggcagcctctagggaggccttgtccgccgtgaagggtgcgacaccgacactgaccgcacaaaatttgatctatcagcggttcagggggtgacacgcatggagggaatcacggtacggacagggacctcagccaaaggtgggagaacttggtactcgttcaactcaccggcaacgacatatgagtacattgtccacaactcatcacttaagaacgtagtcaggggacttgtcgagcgggtcttctgtgttgtggacaagaaaactggtgaactggtccggcccccaaaacctgttaaggggctattcaccaagaagctcggtgacgtcggtcaagtagtgagtcaactcgttggttattgcccccactggacacgtcaagaattcttggcgtcttacaatgggccgcgaaaagccagttacgagcgggctgcgctaacgctagacactctgcccttgcgtgaggaggatgcgcatctgagcacctttgtaaaggcggagaagatcaacgtcactctgaaacctgatcctgccccacgagtgattcagccgcgtggacagcggtacaacattgaggtgggaaggtttctgaaacccctggaaccacgcctaatgaaggcgatcgataagctgtgggggtccaccacagctattaaggggtacacggttgagagagtcggggctatcatgaatgagaaagctaacagatttcgtgagcctgtgtttgtgggtttagatgcctctcggtttgaccaacattgttctgccgaggcccttagatgggaacacagtgtttacaacgacatctttcgatctgagtatctcgcaacactcttacagtggcaggtcaacaatagagggactgcctacactaaagagggtactgtgagttacaaggtagaagggtgccgtatgtctggggacatgaacacgtcgatgggaaattatttaatcatgtcctgcttgatctatgccttttgccgggaagttagactgaaagcggaattggctaactgtggtgacgattgcgtgctgtttttggagaaagaggatcttcacaagcttggcactttaccgcagtggtttgtacgtatgggatatacgatgaaggtggaggagccggtgtatgaggtggagcacattgagttctgccaaatgcgccccattcgcacctccagaggatgggtcatggtcaggcgtccggacactgttctaacaaaggattgttgtgttgtcaggggaggaatgactgaggagcggttgaagggatggcttggtagtatgcgcgatggcggtctcagccttgctggggacgtacccatattgggtgccttctaccggtccttcccatcatacgcttctcaggaagcttccgagtacagcgccccacacaagttccgggcgggtaagcagtacggcgctgtcacagacgagagccggtattccttttggctggcgtttgggctcacacccgacgaccagcttgctgtggagagtgaattgtcaaagatggcgtttcatactcgtccggagcaaaaaggaccgtaccagccctcgctacttgactactgcactagaacctgaccagttcaccagccaccttgactactgcactagaacctgaccagttcaccagccatggcgaggaagaagcggagcaaccaggtatttaaatcctacgaatacgccggaagccacaatttctgcatcgggatttttttcttcgtttagattccaggttcctcctactcccaagaagtagccttgatatccggttcggtacaagatgagacatatcaaattgatcaccaacttcagcaatttcaggacaatggtccccacagtctcgatacttgtcattttcgtaaaaaatcgaacttattatctgcgctatatagtgttccttgatactcagaaccaaggcacacttccggcaataacggacgtgctgtcatcccttgacgtatcatctggatacgaggttcgaacataataagttcgattttttacgaaaatgacaagtatcgagactgtggggaccattgtcctgaaattgctgaagttggtgatcaatttgatatgtctcatcttgtaccgaaccggatatcaaggctacttcttgggagtaggaggaacctggaatctaaacgaagaaaaaaatcccgatgcagaaattgtggcttccggcgtattcgtaggacgcgttcatgtcgataatcagtcttgacggagagtttgattgtcctccttatcaacccacctcatcccgctttcacttcactcacaaaacgcgcaagtctgctatttgtatcggtccttctactttcggcaaattatggcgagtcccgagggctgggtattacaccccaaccgatgtgacctttgtggttacgccacatatctccgagaaagctggcgttatggcgactgtcaaactcatagacgcatccgacatgagcccatcccgagtgctgttcgagaccaaggcgttcaaccttggccatgggacggtactggaggggtctcaattgccgttttgcctgccaatcggggaatatcctatacacttcgaggtcacggtgtcacgatcacagtttcggggagaacggacaatgtactcaacatcactcgagtggcaaatgatgtgttctcccaccccgttatccagggttcgatctgtgttcgcggttgcgcaccaaccagtgttggatgcggtcccgaatttctcaatgaaaaccaaaaagaagtctagcgtcctgtccggtggtaagggtcaagcgacagaaaagaggattttggctggtggtggtacggcccggggagtggttcccccgggatgcgtagcgccagctgaaggaatcccagtaatcgccactatagaagaccactaggagctcatgtactccacgcttcggcggggctataaggagtacatgatacccccccctatctttcacccagcttgctggggccggccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattatcgatgggccccggccgaagctggccgcgggcatgtggtacctaagggcccataggcgcgcccggtgcatgcaagcttgcttcaagggcccgtttgtatcaagtttgtacaaaaaagcaggctggccagaatggcccggaccggcggccgcctgcagctctagagaaacttcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatggcgaggaagaagcggagcaaccaggtacagacgggacagggagtgaggcgagcagcaggggctgtcattacagctcctgtagctaggacccgacaagtgagggcccggccacctaaggtcgaggcgttagcgggcggtggttttcgggtcacccatagggagttgatcactaccattgccaactcggctacataccaggcgaacgggggtattgctggattaaagtacaggatgaatccgacgtacggctccaccttgacgtggtgtccggccttggcatccaacttcgaccagtatgtcttccgcaaattgaccttggaatacgtgccgacgtgtgggacaacggagacggggagggtgggcatctggttcgatagggactctgaagatgacccgcctgctgaccgagtggaattggctagtatgggggtacttgtggagactgctccatggagcggtgtcacactacaggtacccacggacaacaccaagagattctgcctcggcgctggtggcaacacggatgccaaactgatagaccttggtcaaatcggttttagtacgtacgcgggagctgggacgaacgctgtcggtgatctattcgccgagtatgtcgtggatctacactgcccgcaaccgtctggcgcattagtccaaacgttgcgaatcactagtgctggggtgcgaggacctgaagttggaccactatactacaacatgacaaaggcagcaactctcattgacctgacgttcttcacaccaggcacatttctgatctcaataggctgcgcagctacttcgtatacttcggagctagtcctgggaggagccacgctgaactcacgaacactcactgccacaggagccgggttttccgggtcctttaacgtcactgtgaccaagcccttagatggcttacgcatacaaggaaccggattcggtgactgtatgacgtttgctgtccgcgcgagggtggccaactctgttactgtctaggagctcgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaa

SEQ ID NO: 23 - DNA, 686 bp, Artificial Sequence
  Probe
cgtttcagaatggatgaaaaagcagggtgttcctgatcgggtgaacgatgaggtttttattgcaatgtccaaggcactcaatttcataaatcctgatgagctatctatgcagtgcattttgattgctttgaaccgatttcttcaggagaagcatggttctaaaatggcattcttggatggtaatccgcctgaaaggctatgcatgcctattgttgatcacattcggtctaggggtggagaggtccgcctgaattctcgtattaaaaagatagagctgaatcctgatggaactgtaaaacacttcgcacttagtgatggaactcagataactggagatgcttatgtttgtgcaacaccagtcgatatcttcaagcttcttgtacctcaagagtggagtgaaattacttatttcaagaaactggagaagttggtgggagttcctgttatcaatgttcatatatggtttgacagaaaactgaacaacacatatgaccaccttcttttcagcaggagttcacttttaagtgtctatgcagacatgtcagtaacctgcaaggaatactatgacccaaaccgttcaatgctggagttggtctttgctcctgcagacgaatggattggtcgaagtgacactgaaatcatcgatgcaactatggaagagctagccaagttatttcctgatgaaa

SEQ ID NO: 24 - DNA, 211 bp, Diabrotica virgifera virgifera
gataataagttcgattttttacgaaaatgacaagtatcgagactgtggggaccattgtcctgaaattgctgaagttggtgatcaatttgatatgtctcatcttgtaccgaaccggatatcaaggctacttcttgggagtaggaggaacctggaatctaaacgaagaaaaaaatcccgatgcagaaattgtggcttccggcgtattcgtagg

SEQ ID NO: 25 - DNA, 714 bp, Zoanthus sp.
atggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctccggactcagatctcgatag

SEQ ID NO: 26 - DNA, 16 bp, Artificial Sequence
  Probe
ggcgctgctgatgcgg

SEQ ID NO: 27 - DNA, 24 bp, Artificial Sequence
  Probe
ggttcctgcgctatatagtgttcc

SEQ ID NO: 28 - DNA, 28 bp, Artificial Sequence
  Probe
acagcagaatagatttaagttcctactt

SEQ ID NO: 29 - DNA, 44 bp, Artificial Sequence
  Probe
cctccactctgaccttcaatcagaaggtgcaggttcactatggg

SEQ ID NO: 30 - DNA, 18 bp, Artificial Sequence
  Probe
caacttcaaaccggcgca

SEQ ID NO: 31 - DNA, 17 bp, Artificial Sequence
  Probe
ggcctcagacgcgcttg

SEQ ID NO: 32 - DNA, 40 bp, Artificial Sequence
  Probe
cgctatcggcgcactctggacaccacatctgcaaactccg

SEQ ID NO: 33 - DNA, 88 bp, Artificial Sequence
  Probe
ttgatactcagaaccaaggcacacttccggcaataacggacgtgctgtcatcccttgacgtatcatctggatacgaggttctgaatgc

SEQ ID NO: 34 - DNA, 46 bp, Artificial Sequence
  Probe
gatgaggttgaatcactgtgtgccagtgctaccaacctatccaagg

SEQ ID NO: 35 - DNA, 42 bp, Artificial Sequence
  Probe
atgccgtgttcttcttggagttgtctgacaaggttgccacgg

SEQ ID NO: 36 - DNA, 49 bp, Artificial Sequence
  Probe
ggtgtacagctcaagttcactgatgcctagtctttctctgagtgaccgc

SEQ ID NO: 37 - DNA, 19 bp, Artificial Sequence
  Probe
aggctgccaggcagacagg

SEQ ID NO: 38 - DNA, 17 bp, Artificial Sequence
  Probe
ggccgttccagacacgc

SEQ ID NO: 39 - DNA, 18 bp, Artificial Sequence
  Probe
cgcgtcccaagcatcctg

SEQ ID NO: 40 - DNA, 19 bp, Artificial Sequence
  Probe
agtatcgggcatcagccgg

SEQ ID NO: 41 - DNA, 45 bp, Artificial Sequence
  Probe
cctagtccttttattgtacacacgccagtatgcacggactaaggc

SEQ ID NO: 42 - DNA, 41 bp, Artificial Sequence
  Probe
tgagggcgtcaatgaccccctcaaccctatccacagcatcg

SEQ ID NO: 43 - DNA, 82 bp, Artificial Sequence
  Probe
gaccacggcaagtggcagcactcgtactcggtcaccgaaccggacgttgagcactttcatctcgtcgaggataaccttctgg

SEQ ID NO: 44 - DNA, 37 bp, Artificial Sequence
  Probe
gagcgaacctccagtggtccaccatcagcggctgggg

SEQ ID NO: 45 - DNA, 48 bp, Artificial Sequence
  Probe
tagtgtacaaaagacgaacaccactagaagacacacccacaccaacac

SEQ ID NO: 46 - DNA, 35 bp, Artificial Sequence
  Probe
gccagggaacccaccaggccagtgcaagccacggc

SEQ ID NO: 47 - DNA, 90 bp, Artificial Sequence
  Probe
tacacgagagcgtttgccttagtgggcttcaacaagcccacacgactgattgccctcgctgccagtcggcacgcgaaccggttccgcccg

SEQ ID NO: 48 - DNA, 22 bp, Artificial Sequence
  Probe
tgatgctgtcatcctcctcaac

SEQ ID NO: 49 - DNA, 23 bp, Artificial Sequence
  Probe
tattttcttggccaatttgatgt

SEQ ID NO: 50 - DNA, 19 bp, Artificial Sequence
  Probe
agcagcagggccgtacagt

SEQ ID NO: 51 - DNA, 18 bp, Artificial Sequence
  Probe
aaattcgggaccgcatcc

SEQ ID NO: 52 - DNA, 18 bp, Artificial Sequence
  Probe
cctggataacggggtggg

SEQ ID NO: 53 - DNA, 21 bp, Artificial Sequence
  Probe
gacaccgtgacctcgaagtgt

SEQ ID NO: 54 - DNA, 19 bp, Artificial Sequence
  Probe
cccctccagtaccgtccca

SEQ ID NO: 55 - DNA, 20 bp, Artificial Sequence
  Probe
cataacgccagctttctcgg

SEQ ID NO: 56 - DNA, 18 bp, Artificial Sequence
  Probe
tacccagccctcgggact

SEQ ID NO: 57 - DNA, 38 bp, Artificial Sequence
  Probe
aacactggttggtgcgcaaccgcgaacacagatcgaac

SEQ ID NO: 58 - DNA, 47 bp, Artificial Sequence
  Probe
agaacacatcatttgccactcgagtgatgttgagtacattgtccgtt

SEQ ID NO: 59 - DNA, 41 bp, Artificial Sequence
  Probe
ataggatattccccgattggcaggcaaaacggcaattgaga

SEQ ID NO: 60 - DNA, 80 bp, Artificial Sequence
  Probe
tggccaaggttgaacgccttggtctcgaacagcactcgggatgggctcatgtcggatgcgtctatgagtttgacagtcgc

SEQ ID NO: 61 - DNA, 44 bp, Artificial Sequence
  Probe
agatatgtggcgtaaccacaaaggtcacatcggttggggtgtaa

SEQ ID NO: 62 - DNA, 20 bp, Artificial Sequence
  Probe
ctccccgaaactgtgatcgt

SEQ ID NO: 63 - DNA, 21 bp, Artificial Sequence
  Probe
ccctcttggattgggcttcat

SEQ ID NO: 64 - DNA, 24 bp, Artificial Sequence
  Probe
tggggtacttcaaagtcaggatac

SEQ ID NO: 65 - DNA, 23 bp, Artificial Sequence
  Probe
gttgcttacaattccatgttcga

SEQ ID NO: 66 - DNA, 23 bp, Artificial Sequence
  Probe
cagatcttttccatgtcatccca

SEQ ID NO: 67 - DNA, 23 bp, Artificial Sequence
  Probe
gctcattgtagaaggtgtgatgc

SEQ ID NO: 68 - DNA, 20 bp, Artificial Sequence
  Probe
ctcttctggtgcgacacgaa

SEQ ID NO: 69 - DNA, 23 bp, Artificial Sequence
  Probe
gcctctgtaagaagaactgggtg

SEQ ID NO: 70 - DNA, 20 bp, Artificial Sequence
  Probe
tagccttcgggttaagaggg

SEQ ID NO: 71 - DNA, 21 bp, Artificial Sequence
  Probe
tttgcgtcatcttttccctgt

SEQ ID NO: 72 - DNA, 24 bp, Artificial Sequence
  Probe
gaacactgaaggtttcaaacatga

SEQ ID NO: 73 - DNA, 25 bp, Artificial Sequence
  Probe
gcttgaatagcaacatacatagcag

SEQ ID NO: 74 - DNA, 24 bp, Artificial Sequence
  Probe
ccactagcatatagggaaaggaca

SEQ ID NO: 75 - DNA, 22 bp, Artificial Sequence
  Probe
caggacgataccagtggtacgg

SEQ ID NO: 76 - DNA, 24 bp, Artificial Sequence
  Probe
gtgactgacaccatctccagaatc

SEQ ID NO: 77 - DNA, 24 bp, Artificial Sequence
  Probe
gtagccttcatagattgggacagt

SEQ ID NO: 78 - DNA, 18 bp, Artificial Sequence
  Probe
aatggcatgtgggagggc

SEQ ID NO: 79 - DNA, 21 bp, Artificial Sequence
  Probe
gccagcaagatcgagacgaag

SEQ ID NO: 80 - DNA, 21 bp, Artificial Sequence
  Probe
gagggaatcagtgaggtccct

SEQ ID NO: 81 - DNA, 22 bp, Artificial Sequence
  Probe
ccctctcggtgaggatcttcat

SEQ ID NO: 82 - DNA, 23 bp, Artificial Sequence
  Probe
ggcagtggttgtgaaagagtagc

SEQ ID NO: 83 - DNA, 19 bp, Artificial Sequence
  Probe
cacggacaatttcccgctc

SEQ ID NO: 84 - DNA, 22 bp, Artificial Sequence
  Probe
tcctgatgaaattgctgctgat

SEQ ID NO: 85 - DNA, 22 bp, Artificial Sequence
  Probe
accaccttcttttcagcaggag

SEQ ID NO: 86 - DNA, 20 bp, Artificial Sequence
  Probe
tgacccaaaccgttcaatgc

SEQ ID NO: 87 - DNA, 49 bp, Artificial Sequence
  Probe
gtgacactgaaatcatcgatgcaactatggaagagctagccaagttatt

SEQ ID NO: 88 - DNA, 29 bp, Artificial Sequence
  Probe
cagagtaaagcaaagattcttaagtatca

SEQ ID NO: 89 - DNA, 40 bp, Artificial Sequence
  Probe
agccttgccggcctctccaaaggtcacctatcgaaggttt

SEQ ID NO: 90 - DNA, 54 bp, Artificial Sequence
  Probe
ttcctgttatcaatgttcatatatggtttgacagaaaactgaacaacacatatg

SEQ ID NO: 91 - DNA, 51 bp, Artificial Sequence
  Probe
ttcacttttaagtgtctatgcagacatgtcagtaacctgcaaggaatacta

SEQ ID NO: 92 - DNA, 42 bp, Artificial Sequence
  Probe
tggagttggtctttgctcctgcagacgaatggattggtcgaa

SEQ ID NO: 93 - DNA, 47 bp, Artificial Sequence
  Probe
tattgtgaagacaccgagatcggtttacaaaactgtcccaaactgtg

SEQ ID NO: 94 - DNA, 134 bp, Artificial Sequence
  Probe
ctatctagctggtgattacacaaagcagaaatacctggcttctatggaaggtgcagtcctatccgggaagctttgtgcccagtccatagtgcaggattatagcaggctcgcactcaggagccagaaaagcctac

SEQ ID NO: 95 - DNA, 16 bp, Artificial Sequence
  Probe
tgcgtggacggccaca

SEQ ID NO: 96 - DNA, 34 bp, Artificial Sequence
  Probe
atcaacctgtgcgtggtggagggcggccccttgc

SEQ ID NO: 97 - DNA, 20 bp, Artificial Sequence
  Probe
ccgagtacccccaggacatc

SEQ ID NO: 98 - DNA, 34 bp, Artificial Sequence
  Probe
gaggacggcgccgtgtgcatctgcaacgccgaca

SEQ ID NO: 99 - DNA, 14 bp, Artificial Sequence
  Probe
ccgccgacggcccc

SEQ ID NO: 100 - DNA, 18 bp, Artificial Sequence
  Probe
tggcccagtccaagcacg

SEQ ID NO: 101 - DNA, 19 bp, Artificial Sequence
  Probe
agttcgtgatcaccggcga

SEQ ID NO: 102 - DNA, 17 bp, Artificial Sequence
  Probe
cggcaaccgcgtgttca

SEQ ID NO: 103 - DNA, 19 bp, Artificial Sequence
  Probe
ggaccgctccttcctgttc

SEQ ID NO: 104 - DNA, 18 bp, Artificial Sequence
  Probe
tcaccgtgagcgtggagg

SEQ ID NO: 105 - DNA, 19 bp, Artificial Sequence
  Probe
agaagatcatccccgtgcc

SEQ ID NO: 106 - DNA, 18 bp, Artificial Sequence
  Probe
ctgctgaaggacggtggc

SEQ ID NO: 107 - DNA, 41 bp, Artificial Sequence
  Probe
gcctgaccaaggagatgaccatgaagtaccgcatggagggc

SEQ ID NO: 108 - DNA, 34 bp, Artificial Sequence
  Probe
gggcatcggctaccccttcaagggcaagcaggcc

SEQ ID NO: 109 - DNA, 37 bp, Artificial Sequence
  Probe
ccttcgccgaggacatcttgtccgccgccttcatgta

SEQ ID NO: 110 - DNA, 41 bp, Artificial Sequence
  Probe
gtcgactacttcaagaactcctgccccgccggctacacctg

SEQ ID NO: 111 - DNA, 45 bp, Artificial Sequence
  Probe
agaactgcatgtaccacgagtccaagttctacggcgtgaacttcc

SEQ ID NO: 112 - DNA, 40 bp, Artificial Sequence
  Probe
gtgatgaagaagatgaccgacaactgggagccctcctgcg

SEQ ID NO: 113 - DNA, 40 bp, Artificial Sequence
  Probe
caagcagggcatcttgaagggcgacgtgagcatgtacctg

SEQ ID NO: 114 - DNA, 49 bp, Artificial Sequence
  Probe
ttgtccatcttctggattggccaacttaattaatgtatgaaataaaagg

SEQ ID NO: 115 - DNA, 24 bp, Artificial Sequence
  Probe
atgcacacatagtgacatgctaat

SEQ ID NO: 116 - DNA, 23 bp, Artificial Sequence
  Probe
cactataatgtgggcatcaaagt

SEQ ID NO: 117 - protein, 274 aa, Maize White Line Mosaic Virus
MALARGVLSQRVVTAAVDVTFGSVDYSDPRIVAALCDGGLKGRATVRRQIVTALKWLVMVLTWPVRMPAMAIVWCLTWVALMVTRTTRKICCVVSRLYSESSALVRAYWRVYNKRTRAVACTGLVGSLALYGPAAVLVWVCLLVVFVFCTLPADARYYIKLAKKIQDAWDAVEEDDSITPAADGGPLEVRSGRNRFACRLAARAISRVGLLKPTKANALVYQKVILDEMKVLNVRFGDRVRVLPLAVVACLERPDAVDRVEGVIDALTCLPGSL

SEQ ID NO: 118 - protein, 797 aa, Maize White Line Mosaic Virus
  misc_feature (275)..(275): Xaa can be any naturally occurring amino acid
MALARGVLSQRVVTAAVDVTFGSVDYSDPRIVAALCDGGLKGRATVRRQIVTALKWLVMVLTWPVRMPAMAIVWCLTWVALMVTRTTRKICCVVSRLYSESSALVRAYWRVYNKRTRAVACTGLVGSLALYGPAAVLVWVCLLVVFVFCTLPADARYYIKLAKKIQDAWDAVEEDDSITPAADGGPLEVRSGRNRFACRLAARAISRVGLLKPTKANALVYQKVILDEMKVLNVRFGDRVRVLPLAVVACLERPDAVDRVEGVIDALTCLPGSLXGGLVRREGCDTDTDRTKFDLSAVQGVTRMEGITVRTGTSAKGGRTWYSFNSPATTYEYIVHNSSLKNVVRGLVERVFCVVDKKTGELVRPPKPVKGLFTKKLGDVGQVVSQLVGYCPHWTRQEFLASYNGPRKASYERAALTLDTLPLREEDAHLSTFVKAEKINVTLKPDPAPRVIQPRGQRYNIEVGRFLKPLEPRLMKAIDKLWGSTTAIKGYTVERVGAIMNEKANRFREPVFVGLDASRFDQHCSAEALRWEHSVYNDIFRSEYLATLLQWQVNNRGTAYTKEGTVSYKVEGCRMSGDMNTSMGNYLIMSCLIYAFCREVRLKAELANCGDDCVLFLEKEDLHKLGTLPQWFVRMGYTMKVEEPVYEVEHIEFCQMRPIRTSRGWVMVRRPDTVLTKDCCVVRGGMTEERLKGWLGSMRDGGLSLAGDVPILGAFYRSFPSYASQEASEYSAPHKFRAGKQYGAVTDESRYSFWLAFGLTPDDQLAVESELSKMAFHTRPEQKGPYQPSLLDYCTRT

SEQ ID NO: 119 - protein, 332 aa, Maize White Line Mosaic Virus
MARKKRSNQVQTGQGVRRAAGAVITAPVARTRQVRARPPKVEALAGGGFRVTHRELITTIANSATYQANGGIAGLKYRMNPTYGSTLTWCPALASNFDQYVFRKLTLEYVPTCGTTETGRVGIWFDRDSEDDPPADRVELASMGVLVETAPWSGVTLQVPTDNTKRFCLGAGGNTDAKLIDLGQIGFSTYAGAGTNAVGDLFAEYVVDLHCPQPSGALVQTLRITSAGVRGPEVGPLYYNMTKAATLIDLTFFTPGTFLISIGCAATSYTSELVLGGATLNSRTLTATGAGFSGSFNVTVTKPLDGLRIQGTGFGDCMTFAVRARVANSVTV

SEQ ID NO: 120 - protein, 227 aa, Maize White Line Mosaic Virus
MSIISLDGEFDCPPYQPTSSRFHFTHKTRKSAICIGPSTFGKLWRVPRAGYYTPTDVTFVVTPHISEKAGVMATVKLIDASDMSPSRVLFETKAFNLGHGTVLEGSQLPFCLPIGEYPIHFEVTVSRSQFRGERTMYSTSLEWQMMCSPTPLSRVRSVFAVAHQPVLDAVPNFSMKTKKKSSVLSGGKGQATEKRILAGGGTARGVVPPGCVAPAEGIPVIATIEDH

SEQ ID NO: 121 - protein, 138 aa, Maize White Line Mosaic Virus
MASPEGWVLHPNRCDLCGYATYLRESWRYGDCQTHRRIRHEPIPSAVRDQGVQPWPWDGTGGVSIAVLPANRGISYTLRGHGVTITVSGRTDNVLNITRVANDVFSHPVIQGSICVRGCAPTSVGCGPEFLNENQKEV

SEQ ID NO: 122 - protein, 218 aa, Maize White Line Mosaic Virus
MATQLTTRARRATRVSRKGSQPASKQDVKQVVKSILGQSLEHKRANLLLPPTVVNTTGNIYCLTQFVIEGDGISQRTGRVINLEQMVLRYRRTLDTTSANSGFLRYIVFLDTQNQGTLPAITDVLSSLDVSSGYEVLNAQQNRFKFLLDEVESLCASATNLSKASTLTFNQKVQVHYGGAADAATSNRRNAVFFLELSDKVATGPQTRLGVQLKFTDA

SEQ ID NO: 123 - protein, 237 aa, Zoanthus sp.
MAQSKHGLTKEMTMKYRMEGCVDGHKFVITGEGIGYPFKGKQAINLCVVEGGPLPFAEDILSAAFMYGNRVFTEYPQDIVDYFKNSCPAGYTWDRSFLFEDGAVCICNADITVSVEENCMYHESKFYGVNFPADGPVMKKMTDNWEPSCEKIIPVPKQGILKGDVSMYLLLKDGGRLRCQFDTVYKAKSVPRKMPDWHFIQHKLTREDRSDAKNQKWHLTEHAIASGSALPSGLRSR

SEQ ID NO: 124 - protein, 14 aa, Maize White Line Mosaic Virus
MARKKRSNQVQTGQ

SEQ ID NO: 125 - protein, 14 aa, Maize White Line Mosaic Virus
RVSRKGSQPASKQD

SEQ ID NO: 126 - protein, 10 aa, Maize White Line Mosaic Virus
LYSESSALVR

SEQ ID NO: 127 - protein, 12 aa, Maize White Line Mosaic Virus
AAGAVITAPVAR

SEQ ID NO: 128 - protein, 11 aa, Maize White Line Mosaic Virus
LIDASDMSPSR

SEQ ID NO: 129 - protein, 9 aa, Maize White Line Mosaic Virus
TDNVLNITR

SEQ ID NO: 130 - protein, 10 aa, Maize White Line Mosaic Virus
VINLEQMVLR

SEQ ID NO: 131 - DNA, 14 bp, Artificial Sequence
  Probe
gagctcggccggcc

SEQ ID NO: 132 - DNA, 728 bp, Artificial Sequence
  Probe
gagctcatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctccggactcagatctcgatagggccggcc

SEQ ID NO: 133 - DNA, 114 bp, Artificial Sequence
  Probe
gctgacccacgaggaccgcagcgacgcccagaaccagaagtgccacctgaccgagcacgccatcgcctccggctccgccttgccctccggactcagatctcgatagggccggcc

SEQ ID NO: 134 - DNA, 86 bp, Artificial Sequence
  Probe
gagaacctgaagtggcacctgaccgagcacaccatcgccaccggctccgccttgccctccggactcagatctcgatagggccggcc

SEQ ID NO: 135 - DNA, 86 bp, Artificial Sequence
  Probe
  misc_feature (77)..(77): n is a, c, g, or t
  misc_feature (86)..(86): n is a, c, g, or t
gagctcatggcccagtccaagcacggcctgaccaacgacacaaggtggaactgctgctccagagtccgatctgaatncgacgggcn

SEQ ID NO: 136 - DNA, 83 bp, Artificial Sequence
  Probe
  misc_feature (5)..(5): n is a, c, g, or t
  misc_feature (42)..(42): n is a, c, g, or t
gaccncaaggggcacctgacccacggcccgatctccccctgngcccccttatggtcaggagtgagatcacgatacggccagcc

SEQ ID NO: 137 - DNA, 41 bp, Artificial Sequence
  Probe
  misc_feature (23)..(23): n is a, c, g, or t
gagaccccaacgacgcccatganatatcaatccggccggcc

SEQ ID NO: 138 - DNA, 88 bp, Artificial Sequence
  Probe
  misc_feature (8)..(8): n is a, c, g, or t
  misc_feature (13)..(13): n is a, c, g, or t
gaggacancagcnacgccaagaacccgaagaggcacctgaccgcggctcccccctgctctcgggactcctagccctacggactcggat

SEQ ID NO: 139 - DNA, 115 bp, Artificial Sequence
  Probe
agctgacccgcgaggaccgcagcgacgccaagaaccagaagtggcacctgaccgagcacgccatcgcctccggctccgccttgccctccggactcagatctcgatagggccggcc

SEQ ID NO: 140 - protein, 272 aa, Johnsongrass chlorotic stripe mosaic virus
  misc_feature (85)..(85): Xaa can be any naturally occurring amino acid
MDTGILSRRIVTAEVDFQFGSVDYSDPRIVHALCTPGLKERATFGRQIVTALKMAVIALTLPVWWPLRLVWRVIIMGVLWVTRFXTRCTNLIKWCVKETRVTVRAYWNILNKRARGLVVLGCWASFVLYGPYALLLWLGVIVGYIICVLPSNVRYYIELGQKIQDAWDSVEADDTIEAPCNGDILEVRKGRNKFACKLAARAIGRVGLLKATPANALVYQKVILDEMKILNVRFADRVRILPLAVMASLDRPDAVARVEDCVAALTQRGVSL

SEQ ID NO: 141 - protein, 795 aa, Johnsongrass chlorotic stripe mosaic virus
  misc_feature (85)..(85): Xaa can be any naturally occurring amino acid
  misc_feature (273)..(273): Xaa can be any naturally occurring amino acid
MDTGILSRRIVTAEVDFQFGSVDYSDPRIVHALCTPGLKERATFGRQIVTALKMAVIALTLPVWWPLRLVWRVIIMGVLWVTRFXTRCTNLIKWCVKETRVTVRAYWNILNKRARGLVVLGCWASFVLYGPYALLLWLGVIVGYIICVLPSNVRYYIELGQKIQDAWDSVEADDTIEAPCNGDILEVRKGRNKFACKLAARAIGRVGLLKATPANALVYQKVILDEMKILNVRFADRVRILPLAVMASLDRPDAVARVEDCVAALTQRGVSLXGGLVRREGCDTTTDRTNFDLSAVKGVGPTEGLSVRAGTSAKGDRSWYSFNSLATTYEYVVHNGSLKNVCRGLVERVFCVVDKQSGKLVRPPKPKPGVFSAKLGDVGRTVSSIVGYCPHWTRDEFVASYSGPRKASYERAAQTLDTLPLMESDAHLSTFVKAEKINVTLKPDPAPRVIQPRGQRYNIEVGRFLKPLEPRLMKAIDKLWGSTTAIKGYTVEKVGSIFADKASRFRHPVYVGLDASRFDQHCSADALRWEHSVYNDIFRSPYLAELLEWQVHNRGSAYTHEGKVNYRVEGCRMSGDMNTSMGNYLIMSCLIYQFCKEIGLHAELANCGDDCVLFLEKHDLKKLKHLPQWFVKMGYTMKVESPVYELEEVEFCQMHPVRTSRGWVMVRRPDTVMTKDCCVVRGGMTTERLRGWLGAMRDGGLSLAGDVPVLSAFYSSFPQYRNGETSDYDAPHKFRAGKQYGAITAEARYSFWLAFGLTPDDQLAIEGDLSSFKFSLEPQDLVTSMPSLLDYCTRT

SEQ ID NO: 142 - protein, 364 aa, Johnsongrass chlorotic stripe mosaic virus
  misc_feature (250)..(250): Xaa can be any naturally occurring amino acid
MPPQAPTRLGNAWGRRCGTGGLGFQGAWNRLRKRMTNGGGVPMIVGSGGGTVAAPVAVSRQIRSRKPKFTSVKGQVRVTHREYVTQVSGVGSGLFQLNGGLPSGQFRVNPNNAACFPWLLSIASNFDQYRFVNLQLCYVPLCATTEVGRVALFYDKDSGDSGPFERAELANMTHCAETPPWAEVSLTVPCDNVKRYLNDSNVTDLKLVDAGRFGYAVYGGNANTYGDLFIQYTVELSEPQPTAGLIGEVXGNAGTVAGVVQPAYFNFDGFSTTQVAFKPTVVGTYLMTFILDGTGLVLGNVTSSAPEGMSVLDQNVAGSATRVIYVCRVTVQRPGDRLFFNYTGTATFWNLFVVRATRDISITT

SEQ ID NO: 143 - protein, 217 aa, Johnsongrass chlorotic stripe mosaic virus
MSIVNIDGEFEQPQFQDTPSKVYISHKSRKSLVCLGPSVFHKLWKVPKTGFYTPTGVTFVVTPHISESAGVTAVIKLIDMSDMSPSRVLYKSKEFNLGHGLTLEGSQLPFCLPIGEYPIHFEVTVSRSQFQATRTMFSTSLEWHLMYSPTPLSRVRSVFGVAHQPVLEVETNFRMKTKQISSSVVAVLPKQKALGKGLKPVGGTTPGLVTGNCVGTD

SEQ ID NO: 144 - protein, 139 aa, Johnsongrass chlorotic stripe mosaic virus
MEGPKDWVLHPHRCDFCGHATYLRECWRHGSDQVNRHERHEPFPRLVQVQGVQPGTWPDIGRVTTAVLPANRGVSYTLRGHGVTITVSGHENDVFNVARVASDVLTHPVIQGEICVRGSPPTGVGGGNQLPYENQTNIV

SEQ ID NO: 145 - DNA, 54 bp, Maize White Line Mosaic Virus
ccagttcaccagccaccttgactactgcactagaacctgaccagttcaccagcc

SEQ ID NO: 146 - DNA, 36 bp, Maize White Line Mosaic Virus
ctgtggctggctggaggataagaagctaaccacttc

SEQ ID NO: 147 - DNA, 106 bp, Johnsongrass chlorotic stripe mosaic virus
ccagttcaccctaacacgatgtcgatcgtcccagcgaatacgaacagagccctagtgcgcgcaggcactgctcttgcctcaggagccatgacagccatggttccct

SEQ ID NO: 148 - DNA, 38 bp, Johnsongrass chlorotic stripe mosaic virus
tcgcgtcgtgcctgggggattagaagctgaccacttca

SEQ ID NO: 149 - DNA, 6597 bp, Artificial Sequence
  Probe
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccagaatacctcctggatctaaccaatccgtgagagttggccatggccttggctagaggtgttctctcccagcgcgtcgtgacggcggcagttgacgttacttttggtagtgttgactacagtgacccacgcattgtggcagcactgtgtgatgggggtttgaaggggcgggcgaccgtaaggcgtcaaattgtaactgcgctcaaatggctagtgatggtgctcacttggcccgtaaggatgcccgcgatggcgatcgtgtggtgtctgacatgggtagcactgatggtcactcgaaccaccaggaagatctgctgtgtcgttagcaggttgtactccgagtcctccgccttagtccgtgcatactggcgtgtgtacaataaaaggactagggccgtggcttgcactggcctggtgggttccctggcactgtacggccctgctgctgtgttggtgtgggtgtgtcttctagtggtgttcgtcttttgtacactaccggctgatgcccgatactacatcaaattggccaagaaaatacaggatgcttgggacgcggttgaggaggatgacagcatcaccccagccgctgatggtggaccactggaggttcgctccgggcggaaccggttcgcgtgccgactggcagcgagggcaatcagtcgtgtgggcttgttgaagcccactaaggcaaacgctctcgtgtaccagaaggttatcctcgacgagatgaaagtgctcaacgtccggttcggtgaccgagtacgagtgctgccacttgccgtggtcgcgtgtctggaacggcccgatgctgtggatagggttgagggggtcattgacgccctcacctgtctgcctggcagcctctagggaggccttgtccgccgtgaagggtgcgacaccgacactgaccgcacaaaatttgatctatcagcggttcagggggtgacacgcatggagggaatcacggtacggacagggacctcagccaaaggtgggagaacttggtactcgttcaactcaccggcaacgacatatgagtacattgtccacaactcatcacttaagaacgtagtcaggggacttgtcgagcgggtcttctgtgttgtggacaagaaaactggtgaactggtccggcccccaaaacctgttaaggggctattcaccaagaagctcggtgacgtcggtcaagtagtgagtcaactcgttggttattgcccccactggacacgtcaagaattcttggcgtcttacaatgggccgcgaaaagccagttacgagcgggctgcgctaacgctagacactctgcccttgcgtgaggaggatgcgcatctgagcacctttgtaaaggcggagaagatcaacgtcactctgaaacctgatcctgccccacgagtgattcagccgcgtggacagcggtacaacattgaggtgggaaggtttctgaaacccctggaaccacgcctaatgaaggcgatcgataagctgtgggggtccaccacagctattaaggggtacacggttgagagagtcggggctatcatgaatgagaaagctaacagatttcgtgagcctgtgtttgtgggtttagatgcctctcggtttgaccaacattgttctgccgaggcccttagatgggaacacagtgtttacaacgacatctttcgatctgagtatctcgcaacactcttacagtggcaggtcaacaatagagggactgcctacactaaagagggtactgtgagttacaaggtagaagggtgccgtatgtctggggacatgaacacgtcgatgggaaattatttaatcatgtcctgcttgatctatgccttttgccgggaagttagactgaaagcggaattggctaactgtggtgacgattgcgtgctgtttttggagaaagaggatcttcacaagcttggcactttaccgcagtggtttgtacgtatgggatatacgatgaaggtggaggagccggtgtatgaggtggagcacattgagttctgccaaatgcgccccattcgcacctccagaggatgggtcatggtcaggcgtccggacactgttctaacaaaggattgttgtgttgtcaggggaggaatgactgaggagcggttgaagggatggcttggtagtatgcgcgatggcggtctcagccttgctggggacgtacccatattgggtgccttctaccggtccttcccatcatacgcttctcaggaagcttccgagtacagcgccccacacaagttccgggcgggtaagcagtacggcgctgtcacagacgagagccggtattccttttggctggcgtttgggctcacacccgacgaccagcttgctgtggagagtgaattgtcaaagatggcgtttcatactcgtccggagcaaaaaggaccgtaccagccctcgctacttgactactgcactagaacctgaccagttcaccagccaccttgactactgcactagaacctgaccagttcaccagccatggcgaggaagaagcggagcaaccaggtacagacgggacagggagtgaggcgagcagcaggggctgtcattacagctcctgtagctaggacccgacaagtgagggcccggccacctaaggtcgaggcgttagcgggcggtggttttcgggtcacccatagggagttgatcactaccattgccaactcggctacataccaggcgaacgggggtattgctggattaaagtacaggatgaatccgacgtacggctccaccttgacgtggtgtccggccttggcatccaacttcgaccagtatgtcttccgcaaattgaccttggaatacgtgccgacgtgtgggacaacggagacggggagggtgggcatctggttcgatagggactctgaagatgacccgcctgctgaccgagtggaattggctagtatgggggtacttgtggagactgctccatggagcggtgtcacactacaggtacccacggacaacaccaagagattctgcctcggcgctggtggcaacacggatgccaaactgatagaccttggtcaaatcggttttagtacgtacgcgggagctgggacgaacgctgtcggtgatctattcgccgagtatgtcgtggatctacactgcccgcaaccgtctggcgcattagtccaaacgttgcgaatcactagtgctggggtgcgaggacctgaagttggaccactatactacaacatgacaaaggcagcaactctcattgacctgacgttcttcacaccaggcacatttctgatctcaataggctgcgcagctacttcgtatacttcggagctagtcctgggaggagccacgctgaactcacgaacactcactgccacaggagccgggttttccgggtcctttaacgtcactgtgaccaagcccttagatggcttacgcatacaaggaaccggattcggtgactgtatgacgtttgctgtccgcgcgagggtggccaactctgttactgtctagctgtggctggctggaggataagaagctaaccacttcatgtcgataatcagtcttgacggagagtttgattgtcctccttatcaacccacctcatcccgctttcacttcactcacaaaacgcgcaagtctgctatttgtatcggtccttctactttcggcaaattatggcgagtcccgagggctgggtattacaccccaaccgatgtgacctttgtggttacgccacatatctccgagaaagctggcgttatggcgactgtcaaactcatagacgcatccgacatgagcccatcccgagtgctgttcgagaccaaggcgttcaaccttggccatgggacggtactggaggggtctcaattgccgttttgcctgccaatcggggaatatcctatacacttcgaggtcacggtgtcacgatcacagtttcggggagaacggacaatgtactcaacatcactcgagtggcaaatgatgtgttctcccaccccgttatccagggttcgatctgtgttcgcggttgcgcaccaaccagtgttggatgcggtcccgaatttctcaatgaaaaccaaaaagaagtctagcgtcctgtccggtggtaagggtcaagcgacagaaaagaggattttggctggtggtggtacggcccggggagtggttcccccgggatgcgtagcgccagctgaaggaatcccagtaatcgccactatagaagaccactaggacagcatgtactccacgcttcggcggggctataaggagtacatgatacccccccctatctttcacccagcttgctggggtagcccgttaacgccaaaccaacttgtcgttcagaatctattgtcgtgtatttgtattcggacaagtacccaagccaataatgaagagctcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagct

SEQ ID NO: 150 - DNA, 7319 bp, Artificial Sequence
  Probe
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccagaatacctcctggatctaaccaatccgtgagagttggccatggccttggctagaggtgttctctcccagcgcgtcgtgacggcggcagttgacgttacttttggtagtgttgactacagtgacccacgcattgtggcagcactgtgtgatgggggtttgaaggggcgggcgaccgtaaggcgtcaaattgtaactgcgctcaaatggctagtgatggtgctcacttggcccgtaaggatgcccgcgatggcgatcgtgtggtgtctgacatgggtagcactgatggtcactcgaaccaccaggaagatctgctgtgtcgttagcaggttgtactccgagtcctccgccttagtccgtgcatactggcgtgtgtacaataaaaggactagggccgtggcttgcactggcctggtgggttccctggcactgtacggccctgctgctgtgttggtgtgggtgtgtcttctagtggtgttcgtcttttgtacactaccggctgatgcccgatactacatcaaattggccaagaaaatacaggatgcttgggacgcggttgaggaggatgacagcatcaccccagccgctgatggtggaccactggaggttcgctccgggcggaaccggttcgcgtgccgactggcagcgagggcaatcagtcgtgtgggcttgttgaagcccactaaggcaaacgctctcgtgtaccagaaggttatcctcgacgagatgaaagtgctcaacgtccggttcggtgaccgagtacgagtgctgccacttgccgtggtcgcgtgtctggaacggcccgatgctgtggatagggttgagggggtcattgacgccctcacctgtctgcctggcagcctctagggaggccttgtccgccgtgaagggtgcgacaccgacactgaccgcacaaaatttgatctatcagcggttcagggggtgacacgcatggagggaatcacggtacggacagggacctcagccaaaggtgggagaacttggtactcgttcaactcaccggcaacgacatatgagtacattgtccacaactcatcacttaagaacgtagtcaggggacttgtcgagcgggtcttctgtgttgtggacaagaaaactggtgaactggtccggcccccaaaacctgttaaggggctattcaccaagaagctcggtgacgtcggtcaagtagtgagtcaactcgttggttattgcccccactggacacgtcaagaattcttggcgtcttacaatgggccgcgaaaagccagttacgagcgggctgcgctaacgctagacactctgcccttgcgtgaggaggatgcgcatctgagcacctttgtaaaggcggagaagatcaacgtcactctgaaacctgatcctgccccacgagtgattcagccgcgtggacagcggtacaacattgaggtgggaaggtttctgaaacccctggaaccacgcctaatgaaggcgatcgataagctgtgggggtccaccacagctattaaggggtacacggttgagagagtcggggctatcatgaatgagaaagctaacagatttcgtgagcctgtgtttgtgggtttagatgcctctcggtttgaccaacattgttctgccgaggcccttagatgggaacacagtgtttacaacgacatctttcgatctgagtatctcgcaacactcttacagtggcaggtcaacaatagagggactgcctacactaaagagggtactgtgagttacaaggtagaagggtgccgtatgtctggggacatgaacacgtcgatgggaaattatttaatcatgtcctgcttgatctatgccttttgccgggaagttagactgaaagcggaattggctaactgtggtgacgattgcgtgctgtttttggagaaagaggatcttcacaagcttggcactttaccgcagtggtttgtacgtatgggatatacgatgaaggtggaggagccggtgtatgaggtggagcacattgagttctgccaaatgcgccccattcgcacctccagaggatgggtcatggtcaggcgtccggacactgttctaacaaaggattgttgtgttgtcaggggaggaatgactgaggagcggttgaagggatggcttggtagtatgcgcgatggcggtctcagccttgctggggacgtacccatattgggtgccttctaccggtccttcccatcatacgcttctcaggaagcttccgagtacagcgccccacacaagttccgggcgggtaagcagtacggcgctgtcacagacgagagccggtattccttttggctggcgtttgggctcacacccgacgaccagcttgctgtggagagtgaattgtcaaagatggcgtttcatactcgtccggagcaaaaaggaccgtaccagccctcgctacttgactactgcactagaacctgaccagttcaccagccaccttgagctcatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctccggactcagatctcgatagggccggccatggcgaggaagaagcggagcaaccaggtacagacgggacagggagtgaggcgagcagcaggggctgtcattacagctcctgtagctaggacccgacaagtgagggcccggccacctaaggtcgaggcgttagcgggcggtggttttcgggtcacccatagggagttgatcactaccattgccaactcggctacataccaggcgaacgggggtattgctggattaaagtacaggatgaatccgacgtacggctccaccttgacgtggtgtccggccttggcatccaacttcgaccagtatgtcttccgcaaattgaccttggaatacgtgccgacgtgtgggacaacggagacggggagggtgggcatctggttcgatagggactctgaagatgacccgcctgctgaccgagtggaattggctagtatgggggtacttgtggagactgctccatggagcggtgtcacactacaggtacccacggacaacaccaagagattctgcctcggcgctggtggcaacacggatgccaaactgatagaccttggtcaaatcggttttagtacgtacgcgggagctgggacgaacgctgtcggtgatctattcgccgagtatgtcgtggatctacactgcccgcaaccgtctggcgcattagtccaaacgttgcgaatcactagtgctggggtgcgaggacctgaagttggaccactatactacaacatgacaaaggcagcaactctcattgacctgacgttcttcacaccaggcacatttctgatctcaataggctgcgcagctacttcgtatacttcggagctagtcctgggaggagccacgctgaactcacgaacactcactgccacaggagccgggttttccgggtcctttaacgtcactgtgaccaagcccttagatggcttacgcatacaaggaaccggattcggtgactgtatgacgtttgctgtccgcgcgagggtggccaactctgttactgtctagctgtggctggctggaggataagaagctaaccacttcatgtcgataatcagtcttgacggagagtttgattgtcctccttatcaacccacctcatcccgctttcacttcactcacaaaacgcgcaagtctgctatttgtatcggtccttctactttcggcaaattatggcgagtcccgagggctgggtattacaccccaaccgatgtgacctttgtggttacgccacatatctccgagaaagctggcgttatggcgactgtcaaactcatagacgcatccgacatgagcccatcccgagtgctgttcgagaccaaggcgttcaaccttggccatgggacggtactggaggggtctcaattgccgttttgcctgccaatcggggaatatcctatacacttcgaggtcacggtgtcacgatcacagtttcggggagaacggacaatgtactcaacatcactcgagtggcaaatgatgtgttctcccaccccgttatccagggttcgatctgtgttcgcggttgcgcaccaaccagtgttggatgcggtcccgaatttctcaatgaaaaccaaaaagaagtctagcgtcctgtccggtggtaagggtcaagcgacagaaaagaggattttggctggtggtggtacggcccggggagtggttcccccgggatgcgtagcgccagctgaaggaatcccagtaatcgccactatagaagaccactaggacagcatgtactccacgcttcggcggggctataaggagtacatgatacccccccctatctttcacccagcttgctggggtagcccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgc

SEQ ID NO: 151 - DNA, 10959 bp, Artificial Sequence
  Probe
atcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagcaacgagatcatgagccaatcaaagaggagtgatgtagacctaaagcaataatggagccatgacgtaagggcttacgcccatacgaaataattaaaggctgatgtgacctgtcggtctctcagaacctttactttttatgtttggcgtgtatttttaaatttccacggcaatgacgatgtgaccgtcgacccactaaaacattgctttgtcaaaagctaaaaaagatgatgcccgacagccacttgtgtgaagcatgagaagccggtccctccactaagaaaattagtgaagcatcttccagtggtccctccactcacagctcaatcagtgagcaacaggacgaaggaaatgacgtaagccatgacgtctaatcccattcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccagaatacctcctggatctaaccaatccgtgagagttggccatggccttggctagaggtgttctctcccagcgcgtcgtgacggcggcagttgacgttacttttggtagtgttgactacagtgacccacgcattgtggcagcactgtgtgatgggggtttgaaggggcgggcgaccgtaaggcgtcaaattgtaactgcgctcaaatggctagtgatggtgctcacttggcccgtaaggatgcccgcgatggcgatcgtgtggtgtctgacatgggtagcactgatggtcactcgaaccaccaggaagatctgctgtgtcgttagcaggttgtactccgagtcctccgccttagtccgtgcatactggcgtgtgtacaataaaaggactagggccgtggcttgcactggcctggtgggttccctggcactgtacggccctgctgctgtgttggtgtgggtgtgtcttctagtggtgttcgtcttttgtacactaccggctgatgcccgatactacatcaaattggccaagaaaatacaggatgcttgggacgcggttgaggaggatgacagcatcaccccagccgctgatggtggaccactggaggttcgctccgggcggaaccggttcgcgtgccgactggcagcgagggcaatcagtcgtgtgggcttgttgaagcccactaaggcaaacgctctcgtgtaccagaaggttatcctcgacgagatgaaagtgctcaacgtccggttcggtgaccgagtacgagtgctgccacttgccgtggtcgcgtgtctggaacggcccgatgctgtggatagggttgagggggtcattgacgccctcacctgtctgcctggcagcctctagggaggccttgtccgccgtgaagggtgcgacaccgacactgaccgcacaaaatttgatctatcagcggttcagggggtgacacgcatggagggaatcacggtacggacagggacctcagccaaaggtgggagaacttggtactcgttcaactcaccggcaacgacatatgagtacattgtccacaactcatcacttaagaacgtagtcaggggacttgtcgagcgggtcttctgtgttgtggacaagaaaactggtgaactggtccggcccccaaaacctgttaaggggctattcaccaagaagctcggtgacgtcggtcaagtagtgagtcaactcgttggttattgcccccactggacacgtcaagaattcttggcgtcttacaatgggccgcgaaaagccagttacgagcgggctgcgctaacgctagacactctgcccttgcgtgaggaggatgcgcatctgagcacctttgtaaaggcggagaagatcaacgtcactctgaaacctgatcctgccccacgagtgattcagccgcgtggacagcggtacaacattgaggtgggaaggtttctgaaacccctggaaccacgcctaatgaaggcgatcgataagctgtgggggtccaccacagctattaaggggtacacggttgagagagtcggggctatcatgaatgagaaagctaacagatttcgtgagcctgtgtttgtgggtttagatgcctctcggtttgaccaacattgttctgccgaggcccttagatgggaacacagtgtttacaacgacatctttcgatctgagtatctcgcaacactcttacagtggcaggtcaacaatagagggactgcctacactaaagagggtactgtgagttacaaggtagaagggtgccgtatgtctggggacatgaacacgtcgatgggaaattatttaatcatgtcctgcttgatctatgccttttgccgggaagttagactgaaagcggaattggctaactgtggtgacgattgcgtgctgtttttggagaaagaggatcttcacaagcttggcactttaccgcagtggtttgtacgtatgggatatacgatgaaggtggaggagccggtgtatgaggtggagcacattgagttctgccaaatgcgccccattcgcacctccagaggatgggtcatggtcaggcgtccggacactgttctaacaaaggattgttgtgttgtcaggggaggaatgactgaggagcggttgaagggatggcttggtagtatgcgcgatggcggtctcagccttgctggggacgtacccatattgggtgccttctaccggtccttcccatcatacgcttctcaggaagcttccgagtacagcgccccacacaagttccgggcgggtaagcagtacggcgctgtcacagacgagagccggtattccttttggctggcgtttgggctcacacccgacgaccagcttgctgtggagagtgaattgtcaaagatggcgtttcatactcgtccggagcaaaaaggaccgtaccagccctcgctacttgactactgcactagaacctgaccagttcaccagccaccttgactactgcactagaacctgaccagttcaccagccatggcgaggaagaagcggagcaaccaggtacagacgggacagggagtgaggcgagcagcaggggctgtcattacagctcctgtagctaggacccgacaagtgagggcccggccacctaaggtcgaggcgttagcgggcggtggttttcgggtcacccatagggagttgatcactaccattgccaactcggctacataccaggcgaacgggggtattgctggattaaagtacaggatgaatccgacgtacggctccaccttgacgtggtgtccggccttggcatccaacttcgaccagtatgtcttccgcaaattgaccttggaatacgtgccgacgtgtgggacaacggagacggggagggtgggcatctggttcgatagggactctgaagatgacccgcctgctgaccgagtggaattggctagtatgggggtacttgtggagactgctccatggagcggtgtcacactacaggtacccacggacaacaccaagagattctgcctcggcgctggtggcaacacggatgccaaactgatagaccttggtcaaatcggttttagtacgtacgcgggagctgggacgaacgctgtcggtgatctattcgccgagtatgtcgtggatctacactgcccgcaaccgtctggcgcattagtccaaacgttgcgaatcactagtgctggggtgcgaggacctgaagttggaccactatactacaacatgacaaaggcagcaactctcattgacctgacgttcttcacaccaggcacatttctgatctcaataggctgcgcagctacttcgtatacttcggagctagtcctgggaggagccacgctgaactcacgaacactcactgccacaggagccgggttttccgggtcctttaacgtcactgtgaccaagcccttagatggcttacgcatacaaggaaccggattcggtgactgtatgacgtttgctgtccgcgcgagggtggccaactctgttactgtctagctgtggctggctggaggataagaagctaacgcgttcatgtcgataatcagtcttgacggagagtttgattgtcctccttatcaacccacctcatcccgctttcacttcactcacaaaacgcgcaagtctgctatttgtatcggtccttctactttcggcaaattatggcgagtcccgagggctgggtattacaccccaaccgatgtgacctttgtggttacgccacatatctccgagaaagctggcgttatggcgactgtcaaactcatagacgcatccgacatgagcccatcccgagtgctgttcgagaccaaggcgttcaaccttggccatgggacggtactggaggggtctcaattgccgttttgcctgccaatcggggaatatcctatacacttcgaggtcacggtgtcacgatcacagtttcggggagaacggacaatgtactcaacatcactcgagtggcaaatgatgtgttctcccaccccgttatccagggttcgatctgtgttcgcggttgcgcaccaaccagtgttggatgcggtcccgaatttctcaatgaaaaccaaaaagaagtctagcgtcctgtccggtggtaagggtcaagcgacagaaaagaggattttggctggtggtggtacggcccggggagtggttcccccgggatgcgtagcgccagctgaaggaatcccagtaatcgccactatagaagaccactaggagctcatgtactccacgcttcggcggggctataaggagtacatgatacccccccctatctttcacccagcttgctggggccggccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattatcgatgggccccggccgaagctggccgcgggcatgtggtacctaagggcccataggcgcgcccggtgcatgcaagcttgcttcaagggcccgtttgtatcaagtttgtacaaaaaagcaggctggccagaatggcccggaccggcggccgcctgcagctctagagaaacttcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctagggatatttctgctagaaagactcttaatcgttctgaacactttcttgaaagttgcggctgaccaccgtacaggaattctctcgcactagtcgggtttgaagcgcgggtgtatctaggagggtaagcctagagcataaattgtaactaccgcgaataaggtcatggccacccagctcacaacgagagctagaagggcaactcgggtttctcgtaagggatcccagcctgcttctaagcaggacgtgaaacaagttgtcaagtccatccttggacaaagcctggaacacaagagagctaacctactcctgcctcccaccgtggttaacactacagggaacatttactgcctgacgcagtttgtgattgagggcgacggcattagccaaaggaccggtcgtgtcattaacttggagcagatggtgttgcgctatcggcgcactctggacaccacatctgcaaactccgggttcctgcgctatatagtgttccttgatactcagaaccaaggcacacttccggcaataacggacgtgctgtcatcccttgacgtatcatctggatacgaggttctgaatgcacagcagaatagatttaagttcctacttgatgaggttgaatcactgtgtgccagtgctaccaacctatccaaggcctccactctgaccttcaatcagaaggtgcaggttcactatgggggcgctgctgatgcggcaacttcaaaccggcgcaatgccgtgttcttcttggagttgtctgacaaggttgccacggggcctcagacgcgcttgggtgtacagctcaagttcactgatgcctagtcattctctgagtgaccgcctacctggttggggtaagacaccaggaacccctctacgaaatgttcagtcggaagctgagaacctcccggtgcatactgacattgtgagggttcggtaggaagttggccaaaggtttccggatataagccacccggttactgtctaactatccccaaattcggccgtgtctgtcgaaagacagctataggatactctggtgaagccaggaaatgttggagcagggatgtttcagcggtccactggctagcccttgcatggttcttgcatggtcctatagcggtgatgtaacggattccatccactctattattagagctacacgccacacccgagctcgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgc

(19) United States
(12) Patent Application Publication
(10) Pub. No.: US 2020/0165626 A1
(43) Pub. Date: May 28, 2020

(54) VIRUS-INDUCED GENE SILENCING TECHNOLOGY FOR INSECT CONTROL IN MAIZE

(71) Applicant: PIONEER HI-BRED INTERNATIONAL, INC., JOHNSTON, IA (US)

(72) Inventors: JIMENA CARRILLO-TRIPP, JOHNSTON, IA (US); XU HU, JOHNSTON, IA (US)

(73) Assignee: PIONEER HI-BRED INTERNATIONAL, INC., JOHNSTON, IA (US)

(21) Appl. No.: 16/633,314

(22) PCT Filed: Sep. 11, 2018
(86) PCT No.: PCT/US2018/050368

§ 371 Date: Jan. 23, 2020

Related U.S. Application Data
(60) Provisional application No. 62/572,215, filed on Oct. 13, 2017.

(51) Int. Cl. C12N 15/82

(52) U.S. Cl. CPC C12N 15/8286; C12N 15/8203; C12N 15/8218

(57) ABSTRACT

The present invention relates generally to methods of molecular biology and gene silencing to control pests.

### CROSS REFERENCE TO RELATED APPLICATIONS

This application claims benefit of International Application No. PCT/US Serial No. 18/050368 filed on Sep. 11, 2018, which claims priority to U.S. Provisional Application No. 62/572,215, filed Oct. 13, 2017, each of which is hereby incorporated herein in its entirety by reference.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named “5880_SequenceList.txt” created on Oct. 11, 2017, and having a size of 221 kilobytes and is filed concurrently with the specification. The sequence listing contained in this ASCII formatted document is part of the specification and is herein incorporated by reference in its entirety.

### FIELD

The present invention relates generally to methods of molecular biology and gene silencing to control pests.

### BACKGROUND

Plant insect pests are a serious problem in agriculture. They destroy millions of acres of staple crops such as corn, soybeans, peas, and cotton. Yearly, plant insect pests cause over $100 billion dollars in crop damage in the U.S. alone. In an ongoing seasonal battle, farmers must apply billions of gallons of synthetic pesticides to combat these pests. Other methods employed in the past delivered insecticidal activity by microorganisms or genes derived from microorganisms expressed in transgenic plants. For example, certain species of microorganisms of the genus Bacillus are known to possess pesticidal activity against a broad range of insect pests including Lepidoptera, Diptera, Coleoptera, Hemiptera, and others. In fact, microbial pesticides, particularly those obtained from Bacillus strains, have played an important role in agriculture as alternatives to chemical pest control. Agricultural scientists have developed crop plants with enhanced insect resistance by genetically engineering crop plants to produce insecticidal proteins from Bacillus. For example, corn and cotton plants genetically engineered to produce Cry toxins (see, e.g., Aronson (2002) Cell Mol. Life Sci. 59(3):417-425; Schnepf et al. (1998) Microbiol. Mol. Biol. Rev. 62(3):775-806) are now widely used in American agriculture and have provided the farmer with an alternative to traditional insect-control methods. However, in some instances these Bt insecticidal proteins may only protect plants from a relatively narrow range of pests. Thus, novel insect control compositions and methods remain desirable.

### BRIEF SUMMARY

Methods and compositions are provided which employ a silencing element in combination with virus induced gene silencing (VIGS) principle that, when ingested by a plant insect pest, such as a Coleopteran plant pest including a Diabrotica plant pest, is capable of decreasing the expression of a target sequence in the pest. In specific embodiments, the decrease in expression of the target sequence controls the pest and thereby the methods and compositions are capable of limiting damage to a plant, wherein the virus or a modified virus protects the silencing element from nuclease activity or other degradation. Described herein are various target polynucleotides, wherein a decrease in expression of one or more of the sequences in the target pest controls the pest (i.e., has insecticidal activity). Further provided are silencing elements, which when ingested by the pest, decrease the level of expression of one or more of the target polynucleotides. Also described herein are various maize white line mosaic virus (MWLMV) viruses, modified MWLMV viruses, MWLMV satellites, johnsongrass chlorotic stripe mosaic virus (JCSMV), and modified JCSMV viruses. In one embodiment, the MWLMV or modified MWLMV may include a MWLMV coat polypeptide, a MWLMV suppressor of RNA silencing, a satellite MWLMV coat polypeptide, a movement polypeptide, and/or a RNA-directed RNA polymerase polypeptide, and one or more of the polynucleotides encoding the polypeptides set forth in SEQ ID NOS.: 117-122 and 140-144. In some embodiments, the polynucleotides set forth in SEQ ID NOS.: 1-14 encode the polypeptides set forth in SEQ ID NOS.: 117-122 and 140-144. In another embodiment, methods and compositions employ a DNA construct or expression cassette comprising a silencing element and a modified MWLMV virus and/or an MWLMV RNA-dependent RNA polymerase. In some embodiments, a DNA construct of the methods and compositions comprises one of more of the sequences set forth in SEQ ID NOS.: 1-22.

Plants, plant parts, seed, plant cells, bacteria and other host cells comprising the silencing elements, an active variant or fragment thereof and a MWLMV or JCSMV virus or modified MWLMV or JCSMV virus, are also provided. Also provided are formulations of sprayable silencing elements and a MWLMV or JCSMV virus or modified MWLMV or JCSMV virus, for topical applications to pest insects or substrates where pest insects may be found. In another embodiment, the Sprayable formulation comprises a silencing element and a MWLMV or JCSMV virus or modified MWLMV or JCSMV virus expressed in a bacterial host cell. In another embodiment, the formulations and compositions may be applied to a seed as a seed treatment.

In another embodiment, a method for controlling a plant insect pest, such as a Coleopteran plant pest or a Diabrotica plant pest, is provided. In another embodiment, a method for controlling a plant insect pest, such as a Lepidopteran plant pest or a Spodoptera frugiperda plant pest, is provided. In one embodiment, the method comprises feeding to a plant insect pest a composition comprising a silencing element and a MWLMV or JCSMV virus or modified MWLMV or JCSMV virus, wherein the silencing element, when ingested by the pest, reduces the level of a target sequence in the pest and thereby controls the pest. Further provided are methods to protect a plant from a plant insect pest. Such methods comprise introducing into the plant or plant part a disclosed silencing element and a MWLMV or JCSMV virus or modified MWLMV or JCSMV virus. When the plant expressing the silencing element and a MWLMV or JCSMV virus or modified MWLMV or JCSMV virus, is ingested by the pest, the level of the target sequence is decreased and the pest is controlled. Further provided, are methods of using bacteria host cells comprising a silencing element and a MWLMV or JCSMV virus or modified MWLMV or JCSMV virus, for insect for controlling a plant pest.

In another embodiment, a method protects the silencing element from nuclease activity or other degradation, including from the midgut environment of an insect. In another embodiment, methods for screening novel silencing elements are provided. The method comprises feeding to a plant insect a composition comprising a silencing element and a MWLMV or JCSMV virus or modified MWLMV or JCSMV virus, when ingested by the pest, reduces the level of a target sequence in the pest and thereby controls the pest and wherein the composition has increased resistance to nuclease activity and midgut extract. In another embodiment, the method comprises feeding to a plant insect a composition comprising a silencing element and a MWLMV or JCSMV virus or modified MWLMV or JCSMV virus in a host bacterial cell when ingested by the pest, reduces the level of a target sequence in the pest and thereby controls the pest and wherein the composition has increased resistance to nuclease activity and midgut extract. The method may further comprise feeding a different second composition comprising a silencing element and a MWLMV or JCSMV virus or modified MWLMV or JCSMV virus in a host bacterial cell when ingested by the pest, reduces the level of a target sequence in the pest and thereby controls the pest, and comparing the first composition to the first composition to determine the efficacy of a silencing element.

In another embodiment, a method for the production of double stranded RNA is provided. The method comprises using a host cell, such as a bacteria cell, expressing a silencing element and a MWLMV or JCSMV virus or modified MWLMV or JCSMV virus at large scale during fermentation.

### BRIEF DESCRIPTION OF THE DRAWINGS

FIG. 1. Expression cassettes of MWLMV virus for plant expression. A. Diagram of Vector-1 containing wildtype of MWLMV described in Table 2. B. Diagram of Vector-2 containing wildtype of MWLMV satellite virus described in Table 2.

FIG. 2. Modified expression cassettes of MWLMV virus for target expression. A. Diagram of a Vector Design A containing modifications of MWLMV described in Table 2 (Vectors 3 to 9). B. Modified spacer-1 region of vector-6 in Table 2. Silencing element gene of interest target can be inserted between SacI and FseI restriction sites.

FIG. 3. Modified expression cassettes of MWLMV virus for target expression. A. Diagram of a Vector Design B containing modifications of MWLMV and satellite MWLMV described in Table 2 (Vectors 10 to 15). B. Diagram of a Vector Design C containing modifications of MWLMV and satellite MWLMV described in Table 2 (Vectors 16 to 19).

FIG. 4. In vitro transcripts (IVT) of MWLMV and satellite MWLMV. The full genome of MWLMV and satellite were amplified by PCR and used as a template for in vitro transcription. IVT products were analyzed by denaturing agarose electrophoresis. RiboRuler RNA ladder (Thermo Scientific # SM1821) is shown as a size reference.

FIG. 5. Characteristic symptoms induced by MWLMV virus. A, B. Plant inoculated with wt virus (ATCC-PV-489) 35 dpi and 50 dpi respectively. C. A transgenic plant expressing MWLMV. D. Plant inoculated with material concentrated from transgenic plant depicted in C, 15 dpi.

FIG. 6. Western blots of polyclonal antibodies for MWLMV CP and SV-CP. Peptides (MWL-cp-1: MARKKRSNQVQTGQC (SEQ ID NO:124), and Sv-1-1: RVSRKGSQPASKQDC; (SEQ ID NO: 125)) were prepared as KLH conjugates to generate polyclonal antibodies in rabbit. Samples from plants transgenic for MWLMV (plant IDs 2970, 2966, 2998 and 3004) and from a control plant infected with MWLMV and satellite MWLMV (control) concentrated by ultracentrifugation to isolate viral particles are presented. Reference of molecular weight in kDa is shown (MagicMark™ XP Western Protein Standard).

FIG. 7. The expression level in transgenic plants. Quantification of RNA levels in transgenic plants (MWLMV or satellite) compared to infected plants and to transgenic plants expressing a silencing element targeting a gene of interest (SSJ1 Frag1; SEQ ID NO: 24). Two plants transgenic for MWLMV genome or transgenic for satellite were tested independently. Three plants transgenic for MWLMV and satellite were quantified separately. Two plants infected with MWLMV+satellite were tested separately (Error bars, std dev of 3 replicates). The average of 25 plants transgenic for SSJ1 Frag1 tested independently is shown (Error bars, std dev of 25 plants).

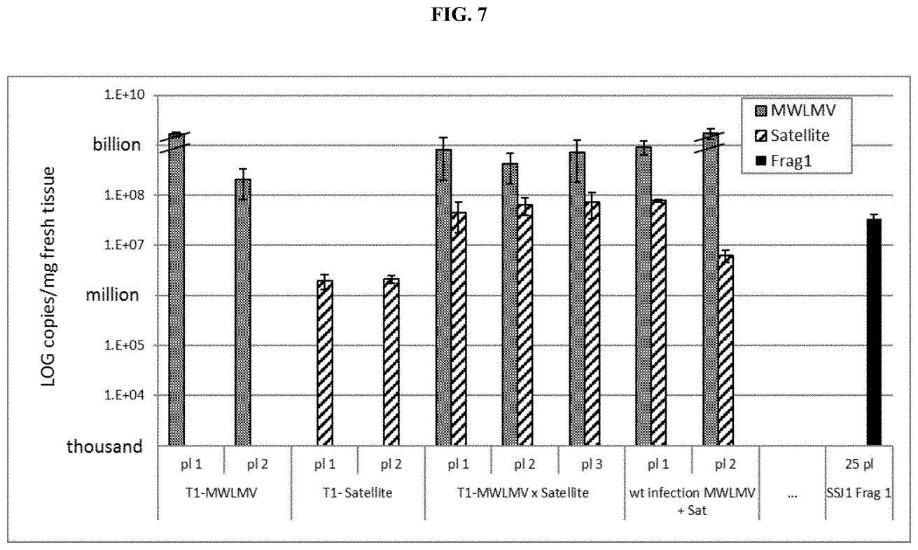

FIG. 8. Shows a sequence alignment of two spacer regions (MWLMV spacer-1 (SEQ ID NO: 145) and JCSMV spacer-1 (SEQ ID NO: 147); and MWLMV spacer-2 (SEQ ID NO: 146) and JCSMV spacer-2 (SEQ ID NO: 148)) between open-reading frames of MWLMV and JCSMV RNA genomes.

### DETAILED DESCRIPTION

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

### I. Overview

The virus induced gene silencing (“VIGS”) principle is based on antiviral responses that target RNAs for degradation and is triggered by the accumulation of double-stranded RNAs (dsRNA) appearing in the infection cycle. By inserting sequence fragments derived from a target “gene-of-interest” (GOI) into a VIGS vector, the corresponding target mRNAs are selectively degraded during virus infection to result in silencing of the targeted gene. There are several possible advantages of VIGS over gene-silencing method involving transgenic plants with inverted repeat construct. (1) The constructs can be assembled by direct cloning in the virus vector and do not involve assembly of inverted repeats that maybe unstable during propagation in the bacterial host or in transformed plants. (2) The procedure is fast, and easy-virus vector constructs can be assembled in a few days and VIGS phenotype developed within 1 or 2 weeks. It is feasible to carry out high-throughput VIGS of many genes in host-pest assay systems. In addition, VIGS may be used as transient seed treatment through Agrobacterium infiltration or direct infection providing rootworm protection in the root.

VIGS can be used as tools for several biotechnological applications. Modified viral genomes known as “viral vectors” have the capacity to copy themselves at high level (“replicons”) in the host cells and to express foreign sequences of interest (Gleba, Tuse, and Giritch, 2014). These characteristics have been exploited in combination with the ability of the virus to induce the RNAi response in the host to develop VIGS vectors. VIGS vectors have been used extensively for plant functional genomics (Velasquez, Chakravarthy, and Martin, 2009; Lu et al. 2003) as well as for the control of plant pests such as insects (Kumar, Pandit, and Baldwin, 2012) and nematodes (Valentine et al., 2007).

Examples of the use of viral vectors for the expression of proteins of interest in planta include the production of fluorescent protein markers (Casper and Holt, 1996); antigens or antibodies (Sainsbury, Liu, and Lomonossoff, 2009). The encapsulation of molecules of interest by viral vectors is done inside the cell, but it can also be achieved outside the cell in vitro systems to package specific drugs (Brown et al., 2002), toxins (Wu, Brown, and Stockley, 1995), or nanomaterials (Douglas and Young, 1998).

“Armored RNA” has been used for producing recombinant virus-like particles that are noninfectious and contain predefined exogenous RNA. This “Armored RNA” has been widely used as controls, standards, or calibrators for the detection of human viruses using reverse transcription-PCR (RT-PCR), real-time RT-PCR, and branched DNA assays. Recently, long RNA has been successfully made with more than 2000 bp ssRNA using a similar MS2 virus-like particle (VLP) expression strategy (Zhan, et al., Journal of Clinical Microbiology, 2009).

Maize white line mosaic virus (MWLMV) belongs to Aureusvirus genus in Tombusviridae family of plant viruses. Its genome consists of linear single-stranded RNA (ssRNA) 4293 nt long (SEQ ID NO: 1), encoding 5 proteins. Open Reading Frame (ORF) 1 (SEQ ID NO: 2) codes for a pre-readthrough of the RNA directed-RNA polymerase (Pre-RNAP) with a predicted molecular weight of 30 kDa. ORF 2 (SEQ ID NO: 3) codes for the viral replicase, RNA directed-RNA polymerase (RNAP) predicted to be 89 kDa. Pre-RNAP and RNAP are involved in replication of viral genome. ORF 3 (SEQ ID NO: 4) codes for the viral coat protein (CP) of 35 kDa. 180 units of CP encapsulate the viral genome to form the MWLMV viral particle of 35 nm diameter. ORF 4 (SEQ ID NO: 5) encodes a movement protein (MP) with a predicted weight of 25 kDa which helps to transport viral genome inside the plant for local and systemic spread. ORF 5 (SEQ ID NO: 6) codes for a putative viral suppressor of RNA silencing (SP) of 15 kDa (Russo M. et al 2008). The genome of Satellite virus (sv) of MWLMV (SEQ ID NO: 7) consists of a linear ssRNA 1168 nt long with a single ORF (SEQ ID NO: 8) which codes for the satellite coat protein (sv-CP) with a predicted molecular weight of 24 kDa (Gingery R. E. and Raymond L. 1985). Sv-CP has no serological no sequence relationship with MWLMV-CP (Zhang L. et al. 1991). 60 units of sv-CP cover the satellite genome to form a satellite particle of ca. 17 nm in diameter (Scholthof, K.-B., et al. 1999).

Johnsongrass chlorotic stripe mosaic virus (JCSMV) is the closest relative of MWLMV reported to this date. It was originally isolated from stunt johnsongrass plants (Sorghum halepense) showing chlorotic stripes (Izadpanah, K. 1998). Virus particles of 30 nm diameter were isolated from symptomatic tissue (Izadpanah, K. 1993). JCSMV belongs to Aureusvirus genus in Tombusviridae family. Its genome consists of linear single-stranded RNA (ssRNA) 4421 nt long (SEQ ID NO: 9, NCBI GenBank Accession No. AJ557804.1), encoding 5 proteins in same order and arrangement than MWLMV. Open Reading Frame ORF 1 (SEQ ID NO: 10) codes for a pre-readthrough of the RNA directed-RNA polymerase (Pre-RNAP) with a predicted molecular weight of 30.5 kDa. ORF 2 (SEQ ID NO: 11) codes for the viral replicase, RNA directed-RNA polymerase (RNAP) predicted to be 89.2 kDa. Pre-RNAP and RNAP are involved in replication of viral genome. ORF 3 (SEQ ID NO: 12) codes for the viral coat protein (CP) of 39 kDa. ORF 4 (SEQ ID NO: 13) encodes a movement protein (MP) of 23.8 kDa predicted to transport viral genome inside the plant. ORF 5 (SEQ ID NO: 14) codes for a small protein of 15.3 kDa, a putative viral suppressor of RNA silencing (SP).

Delivery of a silencing element, such as a double stranded RNA, to a target pest is a prerequisite to developing RNAi as an insect control strategy. The environment of insect midguts can be hostile for a silencing element, where the gut nucleases and pH play a major role among other associated factors. The strong nuclease activities on the dsRNA present in the insect midgut is an important issue to be resolved (Katoch and Thakur, International Journal of Biochemistry and Biotechnology, 2012). It has been reported that nuclease in saliva of Lygus lineolaris digests double stranded ribonucleic acids (Allen and Walker, Journal of Insect Physiology, 2012). It is a technical challenge but very attractive strategy to express various forms of silencing elements inside viral coat proteins for RNAi applications.

As such, methods and compositions are provided which employ one or more silencing elements and a MWLMV or JCSMV virus or modified MWLMV or JCSMV virus, that, when ingested by a plant insect pest, such as a Coleopteran plant pest or a Diabrotica plant pest, is capable of decreasing the expression of a target sequence in the pest and wherein the composition has increased resistance to nuclease activity and midgut extract. In specific embodiments, the decrease in expression of the target sequence controls the pest and thereby the methods and compositions are capable of limiting damage to a plant or plant part. Silencing elements comprising sequences, complementary sequences, active fragments or variants of target polynucleotides are provided which, when ingested by or when contacting the pest, decrease the expression of one or more of the target sequences and thereby controls the pest (i.e., has insecticidal activity). In another embodiment, methods and compositions are provided which employ one or more silencing elements and at least one MWLMV or JCSMV virus or modified MWLMV or JCSMV virus, wherein the MWLMV or JCSMV virus or modified MWLMV or JCSMV virus increases the concentration of the silencing element in a cell. The increased concentration in a cell, when ingested by a plant pest may increase activity of the silencing element towards the plant pest. In certain embodiments, methods and compositions comprise one or more silencing elements and a MWLMV RNA-directed RNA polymerase, wherein the RNAP increases the concentration of the silencing element in a cell. Also disclosed herein are MWLMV or JCSMV virus or modified MWLMV or JCSMV virus encoded by the polynucleotides set forth in SEQ ID NOs: 1-22. The MWLMV or JCSMV virus or modified MWLMV or JCSMV virus may comprise a MWLMV virus, a modified MWLMV virus, a MWLMV satellite, a MWLMV coat polypeptide, a MWLMV suppressor of RNA silencing, a satellite MWLMV coat polypeptide, a MWLMV movement polypeptide, a MWMLV RNA-directed RNA polymerase polypeptide, a JCSMV virus, a modified JCSMV virus, a JCSMV coat polypeptide, a JCSMV suppressor of RNA silencing, a JCSMV movement polypeptide, a JCSMV RNA-directed RNA polymerase polypeptide, and/or any one of the polypeptides set forth in SEQ ID NOS.: 117-122 and 140-144.

In certain embodiments, methods and compositions comprising a VIGS system comprising a MWLMV or JCSMV virus or modified MWLMV or JCSMV virus and a silencing element, wherein the MWLMV or JCSMV virus or modified MWLMV or JCSMV virus comprises a MWLMV, modified MWLMV, and MWLMV satellite, a MWLMV coat polypeptide, a MWLMV suppressor of RNA silencing, a satellite MWLMV coat polypeptide, a movement polypeptide, a MWMLV RNA-directed RNA polymerase polypeptide, a JCSMV, a modified JCSMV, a JCSMV coat polypeptide, a JCSMV suppressor of RNA silencing, a JCSMV movement polypeptide, a JCSMV RNA-directed RNA polymerase polypeptide, and the polypeptides set forth in SEQ ID NOS.: 117-122 and 140-144. In one embodiment, the VIGS system may be used to assess plant functional genomics.

In another embodiment, a silencing element comprises a long dsRNA. The long dsRNA may be at least 50, 100, 150, 200, 250, 300, 350, 400, or 500 nucleotides in length. In another embodiment, the long dsRNA comprises at least 2 different target polynucleotides. In another embodiment, the dsRNA comprises at least 2 different target polynucleotides that target at least 2 different organisms.

As used herein, by “controlling a plant insect pest” or “controls a plant insect pest” is intended any effect on a plant insect pest that results in limiting the damage that the pest causes. Controlling a plant insect pest includes, but is not limited to, killing the pest, inhibiting development of the pest, altering fertility or growth of the pest in such a manner that the pest provides less damage to the plant, or in a manner for decreasing the number of offspring produced, producing less fit pests, producing pests more susceptible to predator attack, other insecticidal proteins or deterring the pests from eating the plant.

Reducing the level of expression of the target polynucleotide or the polypeptide encoded thereby, in the pest results in the suppression, control, and/or killing the invading pest. Reducing the level of expression of the target sequence of the pest will reduce the pest damage by at least about 2% to at least about 6%, at least about 5% to about 50%, at least about 10% to about 60%, at least about 30% to about 70%, at least about 40% to about 80%, or at least about 50% to about 90% or greater. Hence, methods disclosed herein can be utilized to control pests, including but not limited to, Coleopteran plant insect pests or a Diabrotica plant pest.

Certain assays measuring the control of a plant insect pest are commonly known in the art, as are methods to record nodal injury score. See, for example, Oleson et al. (2005) J. Econ. Entomol. 98:1-8. See, for example, the examples below.

Disclosed herein are compositions and methods for protecting plants from a plant insect pest, or inducing resistance in a plant to a plant insect pest, such as Coleopteran plant pests or Diabrotica plant pests or other plant insect pests. Plant insect pests include insects selected from the orders Coleoptera, Diptera, Hymenoptera, Lepidoptera, Mallophaga, Homoptera, Hemiptera Orthroptera, Thysanoptera, Dermaptera, Isoptera, Anoplura, Siphonaptera, Trichoptera, etc., particularly Lepidoptera and Coleoptera.

Those skilled in the art will recognize that not all compositions are equally effective against all pests. Disclosed compositions, including the silencing elements and a MWLMV or JCSMV virus or modified MWLMV or JCSMV virus as disclosed herein, display activity against plant insect pests, which may include economically important agronomic, forest, greenhouse, nursery ornamentals, food and fiber, public and animal health, domestic and commercial structure, household and stored product pests.

As used herein “Coleopteran plant pest” is used to refer to any member of the Coleoptera order. Other plant insect pests that may be targeted by the methods and compositions disclosed herein, but are not limited to Mexican Bean Beetle (Epilachna varivestis), and Colorado potato beetle (Leptinotarsa decemlineata).

As used herein, the term “Diabrotica plant pest” is used to refer to any member of the Diabrotica genus. Accordingly, the compositions and methods may also be useful in protecting plants against any Diabrotica plant pest including, for example, Diabrotica adelpha; Diabrotica amecameca; Diabrotica balteata; Diabrotica barberi; Diabrotica biannularis; Diabrotica cristata; Diabrotica decempunctata; Diabrotica dissimilis; Diabrotica lemniscata; Diabrotica limitata (including, for example, Diabrotica limitata quindecimpuncata); Diabrotica longicornis; Diabrotica nummularis; Diabrotica porracea; Diabrotica scutellata; Diabrotica sexmaculata; Diabrotica speciosa (including, for example, Diabrotica speciosa speciosa); Diabrotica tibialis; Diabrotica undecimpunctata (including, for example, Southern corn rootworm (Diabrotica undecimpunctata), Diabrotica undecimpunctata duodecimnotata; Diabrotica undecimpunctata howardi (spotted cucumber beetle); Diabrotica undecimpunctata undecimpunctata (western spotted cucumber beetle)); Diabrotica virgifera (including, for example, Diabrotica virgifera virgifera (western corn rootworm) and Diabrotica virgifera zeae (Mexican corn rootworm)); Diabrotica viridula; Diabrotica wartensis; Diabrotica sp. JJG335; Diabrotica sp. JJG336; Diabrotica sp. JJG341; Diabrotica sp. JJG356, Diabrotica sp. JJG362; and, Diabrotica sp. JJG365.

In specific embodiments, the Diabrotica plant pest comprises D. virgifera virgifera, D. barberi, D. virgifera zeae, D. speciosa, or D. undecimpunctata howardi.

Larvae of the order Lepidoptera include, but are not limited to, armyworms, cutworms, loopers and heliothines in the family Noctuidae Spodoptera frugiperda JE Smith (fall armyworm); S. exigua Hübner (beet armyworm); S. litura Fabricius (tobacco cutworm, cluster caterpillar); Mamestra configurata Walker (bertha armyworm); M. brassicae Linnaeus (cabbage moth); Agrotis ipsilon Hufnagel (black cutworm); A. orthogonia Morrison (western cutworm); A. subterranea Fabricius (granulate cutworm); Alabama argillacea Hübner (cotton leaf worm); Trichoplusia ni Hübner (cabbage looper); Pseudoplusia includens Walker (soybean looper); Anticarsia gemmatalis Hfibner (velvetbean caterpillar); Hypena scabra Fabricius (green cloverworm); Heliothis virescens Fabricius (tobacco budworm); Pseudaletia unipuncta Haworth (armyworm); Athetis mindara Barnes and Mcdunnough (rough skinned cutworm); Euxoa messoria Harris (darksided cutworm); Earias insulana Boisduval (spiny bollworm); E. vittella Fabricius (spotted bollworm); Helicoverpa armigera Hübner (American bollworm); H. zea Boddie (corn earworm or cotton bollworm); Melanchra picta Harris (zebra caterpillar); Egira (Xylomyges) curialis Grote (citrus cutworm); borers, casebearers, webworms, coneworms, and skeletonizers from the family Pyralidae Ostrinia nubilalis Hübner (European corn borer); Amyelois transitella Walker (naval orangeworm); Anagasta kuehniella Zeller (Mediterranean flour moth); Cadra cautella Walker (almond moth); Chilo suppressalis Walker (rice stem borer); C. partellus, (sorghum borer); Corcyra cephalonica Stainton (rice moth); Crambus caliginosellus Clemens (corn root webworm); C. teterrellus Zincken (bluegrass webworm); Cnaphalocrocis medinalis Guenee (rice leaf roller); Desmia funeralis Hübner (grape leaffolder); Diaphania hyalinata Linnaeus (melon worm); D. nitidalis Stoll (pickleworm); Diatraea grandiosella Dyar (southwestern corn borer), D. saccharalis Fabricius (surgarcane borer); Eoreuma loftini Dyar (Mexican rice borer); Ephestia elutella Hübner (tobacco (cacao) moth); Galleria mellonella Linnaeus (greater wax moth); Herpetogramma licarsisalis Walker (sod webworm); Homoeosoma electellum Hulst (sunflower moth); Elasmopalpus lignosellus Zeller (lesser cornstalk borer); Achroia grisella Fabricius (lesser wax moth); Loxostege sticticalis Linnaeus (beet webworm); Orthaga thyrisalis Walker (tea tree web moth); Maruca testulalis Geyer (bean pod borer); Plodia interpunctella Hübner (Indian meal moth); Scirpophaga incertulas Walker (yellow stem borer); Udea rubigalis Guenee (celery leaftier); and leafrollers, budworms, seed worms and fruit worms in the family Tortricidae Acleris gloverana Walsingham (Western blackheaded budworm); A. variana Fernald (Eastern blackheaded budworm); Archips argyrospila Walker (fruit tree leaf roller); A. rosana Linnaeus (European leaf roller); and other Archips species, Adoxophyes orana Fischer von Rosslerstamm (summer fruit tortrix moth); Cochylis hospes Walsingham (banded sunflower moth); Cydia latiferreana Walsingham (filbertworm); C. pomonella Linnaeus (coding moth); Platynota flavedana Clemens (variegated leafroller); P. stultana Walsingham (omnivorous leafroller); Lobesia botrana Denis & Schiffermuller (European grape vine moth); Spilonota ocellana Denis & Schiffermuller (eyespotted bud moth); Endopiza viteana Clemens (grape berry moth); Eupoecilia ambiguella Hubner (vine moth); Bonagota salubricola Meyrick (Brazilian apple leafroller); Grapholita molesta Busck (oriental fruit moth); Suleima helianthana Riley (sunflower bud moth); Argyrotaenia spp.; Choristoneura spp.

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guerin-Meneville (Chinese Oak Tussah Moth); Bombyx mori Linnaeus (Silkworm); Bucculatrix thurberiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hubner (elm spanworm); Erannis tiliaria Harris (linden looper); Euproctis chrysorrhoea Linnaeus (browntail moth); Harrisina americana Guerin-Meneville (grapeleaf skeletonizer); Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Manduca quinquemaculata Haworth (five spotted hawk moth, tomato hornworm); M. sexta Haworth (tomato hornworm, tobacco hornworm); Operophtera brumata Linnaeus (winter moth); Paleacrita vernata Peck (spring cankerworm); Papilio cresphontes Cramer (giant swallowtail orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval and Leconte (Southern cabbageworm); Sabulodes aegrotata Guenee (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tuta absoluta Meyrick (tomato leafminer); Yponomeuta padella Linnaeus (ermine moth); Heliothis subflexa Guenee; Malacosoma spp. and Orgyia spp.

Of interest are larvae and adults of the order Coleoptera including weevils from the families Anthribidae, Bruchidae and Curculionidae (including, but not limited to: Anthonomus grandis Boheman (boll weevil); Lissorhoptrus oryzophilus Kuschel (rice water weevil); Sitophilus granarius Linnaeus (granary weevil); S. oryzae Linnaeus (rice weevil); Hypera punctata Fabricius (clover leaf weevil); Cylindrocopturus adspersus LeConte (sunflower stem weevil); Smicronyx fulvus LeConte (red sunflower seed weevil); S. sordidus LeConte (gray sunflower seed weevil); Sphenophorus maidis Chittenden (maize billbug)); flea beetles, cucumber beetles, rootworms, leaf beetles, potato beetles and leafminers in the family Chrysomelidae (including, but not limited to: Leptinotarsa decemlineata Say (Colorado potato beetle); Diabrotica virgifera virgifera LeConte (western corn rootworm); D. barberi Smith and Lawrence (northern corn rootworm); D. undecimpunctata howardi Barber (southern corn rootworm); Chaetocnema pulicaria Melsheimer (corn flea beetle); Phyllotreta cruciferae Goeze (Crucifer flea beetle); Phyllotreta striolata (stripped flea beetle); Colaspis brunnea Fabricius (grape colaspis); Oulema melanopus Linnaeus (cereal leaf beetle); Zygogramma exclamationis Fabricius (sunflower beetle)); beetles from the family Coccinellidae (including, but not limited to: Epilachna varivestis Mulsant (Mexican bean beetle)); chafers and other beetles from the family Scarabaeidae (including, but not limited to: Popillia japonica Newman (Japanese beetle); Cyclocephala borealis Arrow (northern masked chafer, white grub); C. immaculata Olivier (southern masked chafer, white grub); Rhizotrogus majalis Razoumowsky (European chafer); Phyllophaga crinita Burmeister (white grub); Ligyrus gibbosus De Geer (carrot beetle)); carpet beetles from the family Dermestidae; wireworms from the family Elateridae, Eleodes spp., Melanotus spp.; Conoderus spp.; Limonius spp.; Agriotes spp.; Ctenicera spp.; Aeolus spp.; bark beetles from the family Scolytidae and beetles from the family Tenebrionidae.

Adults and immatures of the order Diptera are of interest, including leafminers Agromyza parvicornis Loew (corn blotch leafminer); midges (including, but not limited to: Contarinia sorghicola Coquillett (sorghum midge); Mayetiola destructor Say (Hessian fly); Sitodiplosis mosellana Gehin (wheat midge); Neolasioptera murtfeldtiana Felt, (sunflower seed midge)); fruit flies (Tephritidae), Oscinella frit Linnaeus (fruit flies); maggots (including, but not limited to: Delia platura Meigen (seedcorn maggot); D. coarctata Fallen (wheat bulb fly) and other Delia spp., Meromyza americana Fitch (wheat stem maggot); Musca domestica Linnaeus (house flies); Fannia canicularis Linnaeus, F. femoralis Stein (lesser house flies); Stomoxys calcitrans Linnaeus (stable flies)); face flies, horn flies, blow flies, Chrysomya spp.; Phormia spp. and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds) and other Brachycera, mosquitoes Aedes spp.; Anopheles spp.; Culex spp.; black flies Prosimulium spp.; Simulium spp.; biting midges, sand flies, sciarids, and other Nematocera.

Included as insects of interest are adults and nymphs of the orders Hemiptera and Homoptera such as, but not limited to, adelgids from the family Adelgidae, plant bugs from the family Miridae, cicadas from the family Cicadidae, leafhoppers, Empoasca spp.; from the family Cicadellidae, planthoppers from the families Cixiidae, Flatidae, Fulgoroidea, Issidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Asterolecanidae, Coccidae, Dactylopiidae, Diaspididae, Eriococcidae Ortheziidae, Phoenicococcidae and Margarodidae, lace bugs from the family Tingidae, stink bugs from the family Pentatomidae, cinch bugs, Blissus spp.; and other seed bugs from the family Lygaeidae, spittlebugs from the family Cercopidae squash bugs from the family Coreidae and red bugs and cotton stainers from the family Pyrrhocoridae.

Agronomically important members from the order Homoptera further include, but are not limited to: Acyrthisiphon pisum Harris (pea aphid); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacorthum solani Kaltenbach (foxglove aphid); Chaetosiphon fragaefolii Cockerell (strawberry aphid); Diuraphis noxia Kurdjumov/Mordvilko (Russian wheat aphid); Dysaphis plantaginea Paaserini (rosy apple aphid); Eriosoma lanigerum Hausmann (woolly apple aphid); Brevicoryne brassicae Linnaeus (cabbage aphid); Hyalopterus pruni Geoffroy (mealy plum aphid); Lipaphis erysimi Kaltenbach (turnip aphid); Metopolophium dirrhodum Walker (cereal aphid); Macrosiphum euphorbiae Thomas (potato aphid); Myzus persicae Sulzer (peach-potato aphid, green peach aphid); Nasonovia ribisnigri Mosley (lettuce aphid); Pemphigus spp. (root aphids and gall aphids); Rhopalosiphum maidis Fitch (corn leaf aphid); R. padi Linnaeus (bird cherry-oat aphid); Schizaphis graminum Rondani (greenbug); Sipha flava Forbes (yellow sugarcane aphid); Sitobion avenae Fabricius (English grain aphid); Therioaphis maculata Buckton (spotted alfalfa aphid); Toxoptera aurantii Boyer de Fonscolombe (black citrus aphid) and T. citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly); B. argentifolii Bellows & Perring (silverleaf whitefly); Dialeurodes citri Ashmead (citrus whitefly); Trialeurodes abutiloneus (bandedwinged whitefly) and T. vaporariorum Westwood (greenhouse whitefly); Empoasca fabae Harris (potato leafhopper); Laodelphax striatellus Fallen (smaller brown planthopper); Macrolestes quadrilineatus Forbes (aster leafhopper); Nephotettix cinticeps Uhler (green leafhopper); N. nigropictus Stl (rice leafhopper); Nilaparvata lugens Stl (brown planthopper); Peregrinus maidis Ashmead (corn planthopper); Sogatella furcifera Horvath (white-backed planthopper); Sogatodes orizicola Muir (rice delphacid); Typhlocyba pomaria McAtee (white apple leafhopper); Erythroneoura spp. (grape leafhoppers); Magicicada septendecim Linnaeus (periodical cicada); Icerya purchasi Maskell (cottony cushion scale); Quadraspidiotus perniciosus Comstock (San Jose scale); Planococcus citri Risso (citrus mealybug); Pseudococcus spp. (other mealybug complex); Cacopsylla pyricola Foerster (pear psylla); Trioza diospyri Ashmead (persimmon psylla).

Agronomically important species of interest from the order Hemiptera include, but are not limited to: Acrosternum hilare Say (green stink bug); Anasa tristis De Geer (squash bug); Blissus leucopterus leucopterus Say (chinch bug); Corythuca gossypii Fabricius (cotton lace bug); Cyrtopeltis modesta Distant (tomato bug); Dysdercus suturellus Herrich-Schaffer (cotton stainer); Euschistus servus Say (brown stink bug); E. variolarius Palisot de Beauvois (one-spotted stink bug); Graptostethus spp. (complex of seed bugs); Leptoglossus corculus Say (leaf-footed pine seed bug); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Lygocoris pabulinus Linnaeus (common green capsid); Nezara viridula Linnaeus (southern green stink bug); Oebalus pugnax Fabricius (rice stink bug); Oncopeltus fasciatus Dallas (large milkweed bug); Pseudatomoscelis seriatus Reuter (cotton fleahopper).

Furthermore, embodiments may be effective against Hemiptera such, Calocoris norvegicus Gmelin (strawberry bug); Orthops campestris Linnaeus; Plesiocoris rugicollis Fallen (apple capsid); Cyrtopeltis modestus Distant (tomato bug); Cyrtopeltis notatus Distant (suckfly); Spanagonicus albofasciatus Reuter (whitemarked fleahopper); Diaphnocoris chlorionis Say (honeylocust plant bug); Labopidicola allii Knight (onion plant bug); Pseudatomoscelis seriatus Reuter (cotton fleahopper); Adelphocoris rapidus Say (rapid plant bug); Poecilocapsus lineatus Fabricius (four-lined plant bug); Nysius ericae Schilling (false chinch bug); Nysius raphanus Howard (false chinch bug); Nezara viridula Linnaeus (Southern green stink bug); Eurygaster spp.; Coreidae spp.; Pyrrhocoridae spp.; Tinidae spp.; Blostomatidae spp.; Reduviidae spp. and Cimicidae spp.

Also included are adults and larvae of the order Acari (mites) such as Aceria tosichella Keifer (wheat curl mite); Petrobia latens Muiller (brown wheat mite); spider mites and red mites in the family Tetranychidae, Panonychus ulmi Koch (European red mite); Tetranychus urticae Koch (two spotted spider mite); (T. mcdanieli McGregor (McDaniel mite); T. cinnabarinus Boisduval (carmine spider mite); T. turkestani Ugarov & Nikolski (strawberry spider mite); flat mites in the family Tenuipalpidae, Brevipalpus lewisi McGregor (citrus flat mite); rust and bud mites in the family Eriophyidae and other foliar feeding mites and mites important in human and animal health, i.e., dust mites in the family Epidermoptidae, follicle mites in the family Demodicidae, grain mites in the family Glycyphagidae, ticks in the order Ixodidae. Ixodes scapularis Say (deer tick); I. holocyclus Neumann (Australian paralysis tick); Dermacentor variabilis Say (American dog tick); Amblyomma americanum Linnaeus (lone star tick) and scab and itch mites in the families Psoroptidae, Pyemotidae and Sarcoptidae.

Insect pests of the order Thysanura are of interest, such as Lepisma saccharina Linnaeus (silverfish); Thermobia domestica Packard (firebrat).

Insect pest of interest include the superfamily of stink bugs and other related insects including but not limited to species belonging to the family Pentatomidae (Nezara viridula, Halyomorpha halys, Piezodorus guildini, Euschistus servus, Acrosternum hilare, Euschistus heros, Euschistus tristigmus, Acrosternum hilare, Dichelops furcatus, Dichelops melacanthus, and Bagrada hilaris (Bagrada Bug)), the family Plataspidae (Megacopta cribraria—Bean plataspid) and the family Cydnidae (Scaptocoris castanea—Root stink bug) and Lepidoptera species including but not limited to: diamond-back moth, e.g., Helicoverpa zea Boddie; soybean looper, e.g., Pseudoplusia includens Walker and velvet bean caterpillar e.g., Anticarsia gemmatalis Hübner.

### II. Target Sequences

As used herein, a “target sequence” or “target polynucleotide” comprises any sequence in the pest that one desires to reduce the level of expression thereof. In specific embodiments, decreasing the level of the target sequence in the pest controls the pest. For instance, the target sequence may be essential for growth and development. In another embodiment, the target sequence may influence fecundity or reproduction. While the target sequence can be expressed in any tissue of the pest, in specific embodiments, the sequences targeted for suppression in the pest are expressed in cells of the gut tissue of the pest, cells in the midgut of the pest, and cells lining the gut lumen or the midgut. Such target sequences may be involved in, for example, gut cell metabolism, growth or differentiation. As exemplified elsewhere herein, decreasing the level of expression of one or more of these target sequences in a Coleopteran plant pest or a Diabrotica plant pest controls the pest.

### III. Silencing Elements

By “silencing element” is intended a polynucleotide which when contacted by or ingested by a plant insect pest, is capable of reducing or eliminating the level or expression of a target polynucleotide or the polypeptide encoded thereby. Accordingly, it is to be understood that “silencing element,” as used herein, comprises polynucleotides such as RNA constructs, double stranded RNA (dsRNA), hairpin RNA, siRNA, miRNA, amiRNA, and sense and/or antisense RNA. In certain embodiments, the silencing element is complementary to the target sequence. In one embodiment, the silencing element employed can reduce or eliminate the expression level of the target sequence by influencing the level of the target RNA transcript or, alternatively, by influencing translation and thereby affecting the level of the encoded polypeptide. Methods to assay for functional silencing elements that are capable of reducing or eliminating the level of a sequence of interest are disclosed elsewhere herein. A single polynucleotide employed in the disclosed methods can comprise one or more silencing elements to the same or different target polynucleotides. The silencing element can be produced in vivo (i.e., in a host cell such as a plant or microorganism) or in vitro.

In certain embodiments, a silencing element may comprise a chimeric construction molecule comprising two or more disclosed sequences or portions thereof. For example, the chimeric construction may be a hairpin or dsRNA as disclosed herein. A chimera may comprise two or more disclosed sequences or portions thereof. In one embodiment, a chimera contemplates two complementary sequences set forth herein, or portions thereof, having some degree of mismatch between the complementary sequences such that the two sequences are not perfect complements of one another. Providing at least two different sequences in a single silencing element may allow for targeting multiple genes using one silencing element and/or for example, one expression cassette. Targeting multiple genes may allow for slowing or reducing the possibility of resistance by the pest. In addition, providing multiple targeting abilities in one expressed molecule may reduce the expression burden of the transformed plant or plant product, or provide topical treatments that are capable of targeting multiple hosts with one application.

In certain embodiments, while the silencing element controls pests, preferably the silencing element has no effect on the normal plant or plant part.

As discussed in further detail below, silencing elements can include, but are not limited to, a sense suppression element, an antisense suppression element, a double stranded RNA, a siRNA, an amiRNA, a miRNA, or a hairpin suppression element. In an embodiment, silencing elements may comprise a chimera where two or more disclosed sequences or active fragments or variants, or complements thereof, are found in the silencing element. In various embodiments, a disclosed sequence or active fragment or variant, or complement thereof, may be present as more than one copy in a DNA construct, silencing element, DNA molecule or RNA molecule. In a hairpin or dsRNA molecule, the location of a sense or antisense sequence in the molecule, for example, in which sequence is transcribed first or is located on a particular terminus of the RNA molecule, is not limiting to the disclosed sequences, and the dsRNA is not to be limited by disclosures herein of a particular location for such a sequence. The silencing element can further comprise additional sequences that advantageously effect transcription and/or the stability of a resulting transcript. For example, the silencing elements can comprise at least one thymine residue at the 3′ end. This can aid in stabilization. Thus, the silencing elements can have at least 1, 2, 3, 4, 5, 6, 7, 8, 9, 10 or more thymine residues at the 3′ end. As discussed in further detail below, enhancer suppressor elements can also be employed in conjunction with the silencing elements disclosed herein.

By “reduces” or “reducing” the expression level of a polynucleotide or a polypeptide encoded thereby is intended to mean, the polynucleotide or polypeptide level of the target sequence is statistically lower than the polynucleotide level or polypeptide level of the same target sequence in an appropriate control pest which is not exposed to (i.e., has not ingested or come into contact with) the silencing element. In particular embodiments, methods and/or compositions disclosed herein reduce the polynucleotide level and/or the polypeptide level of the target sequence in a plant insect pest to less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control pest. In some embodiments, a silencing element has substantial sequence identity to the target polynucleotide, typically greater than about 65% sequence identity, greater than about 85% sequence identity, about 90%, 91%, 92%, 93%, 94%, 95%, 96%, 97%, 98% or 99% sequence identity. Furthermore, a silencing element can be complementary to a portion of the target polynucleotide. Generally, target sequences of at least 15, 16, 17, 18, 19, 20, 22, 25, 50, 100, 200, 300, 400, 450 continuous nucleotides or greater of the sequence may be used. Methods to assay for the level of the RNA transcript, the level of the encoded polypeptide, or the activity of the polynucleotide or polypeptide are discussed elsewhere herein.

i. Sense Suppression Elements

As used herein, a “sense suppression element” comprises a polynucleotide designed to express an RNA molecule corresponding to at least a part of a target messenger RNA in the “sense” orientation. Expression of the RNA molecule comprising the sense suppression element reduces or eliminates the level of the target polynucleotide or the polypeptide encoded thereby. The polynucleotide comprising the sense suppression element may correspond to all or part of the sequence of the target polynucleotide, all or part of the 5′ and/or 3′ untranslated region of the target polynucleotide, all or part of the coding sequence of the target polynucleotide, or all or part of both the coding sequence and the untranslated regions of the target polynucleotide.

Typically, a sense suppression element has substantial sequence identity to the target polynucleotide, typically greater than about 65% sequence identity, greater than about 85% sequence identity, about 90%, 91%, 92%, 93%, 94%, 95%, 96%, 97%, 98% or 99% sequence identity. See, U.S. Pat. Nos. 5,283,184 and 5,034,323. The sense suppression element can be any length so long as it allows for the suppression of the targeted sequence. The sense suppression element can be, for example, 15, 16, 17, 18, 19, 20, 22, 25, 30, 50, 100, 150, 200, 250, 300, 350, 400, 450, 500, 600, 700, 900, 1000, 1100, 1200, 1300 nucleotides or longer. In other embodiments, the sense suppression element can be, for example, about 15-25, 19-35, 19-50, 25-100, 100-150, 150-200, 200-250, 250-300, 300-350, 350-400, 450-500, 500-550, 550-600, 600-650, 650-700, 700-750, 750-800, 800-850, 850-900, 900-950, 950-1000, 1000-1050, 1050-1100, 1100-1200, 1200-1300, 1300-1400, 1400-1500, 1500-1600, 1600-1700, 1700-1800 nucleotides or longer of the target polynucleotides.

ii. Antisense Suppression Elements

As used herein, an “antisense suppression element” comprises a polynucleotide which is designed to express an RNA molecule complementary to all or part of a target messenger RNA. Expression of the antisense RNA suppression element reduces or eliminates the level of the target polynucleotide. The polynucleotide for use in antisense suppression may correspond to all or part of the complement of the sequence encoding the target polynucleotide, all or part of the complement of the 5′ and/or 3′ untranslated region of the target polynucleotide, all or part of the complement of the coding sequence of the target polynucleotide, or all or part of the complement of both the coding sequence and the untranslated regions of the target polynucleotide. In addition, the antisense suppression element may be fully complementary (i.e., 100% identical to the complement of the target sequence) or partially complementary (i.e., less than 100% identical to the complement of the target sequence) to the target polynucleotide. In certain embodiments, the antisense suppression element comprises at least 85%, 90%, 91%, 92%, 93%, 94%, 95%, 96%, 97%, 98%, or 99% sequence complementarity to the target polynucleotide. Antisense suppression may be used to inhibit the expression of multiple proteins in the same plant. See, for example, U.S. Pat. No. 5,942,657. Furthermore, the antisense suppression element can be complementary to a portion of the target polynucleotide. Generally, sequences of at least 15, 16, 17, 18, 19, 20, 22, 25, 50, 100, 200, 300, 400, 450 nucleotides or greater of the sequence may be used. Methods for using antisense suppression to inhibit the expression of endogenous genes in plants are described, for example, in Liu et al (2002) Plant Physiol. 129:1732-1743 and U.S. Pat. No. 5,942,657.

iii. Double Stranded RNA Suppression Element

A “double stranded RNA” or “dsRNA,” comprises at least one transcript that is capable of forming a dsRNA either before or after ingestion by a plant insect pest. Thus, a “dsRNA silencing element” includes a dsRNA, a transcript or polyribonucleotide capable of forming a dsRNA or more than one transcript or polyribonucleotide capable of forming a dsRNA. “Double stranded RNA” or “dsRNA” refers to a polyribonucleotide structure formed either by a single self-complementary RNA molecule or a polyribonucleotide structure formed by the expression of at least two distinct RNA strands. The dsRNA molecule(s) employed in the disclosed methods and compositions mediate the reduction of expression of a target sequence, for example, by mediating RNA interference “RNAi” or gene silencing in a sequence-specific manner. In various embodiments, the dsRNA is capable of reducing or eliminating the level or expression of a target polynucleotide or the polypeptide encoded thereby in a plant insect pest.

The dsRNA can reduce or eliminate the expression level of the target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). For example, see Verdel et al. (2004) Science 303:672-676; Pa1-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237. Methods to assay for functional dsRNA that are capable of reducing or eliminating the level of a sequence of interest are disclosed elsewhere herein. Accordingly, as used herein, the term “dsRNA” is meant to encompass other terms used to describe nucleic acid molecules that are capable of mediating RNA interference or gene silencing, including, for example, short-interfering RNA (siRNA), double-stranded RNA (dsRNA), micro-RNA (miRNA), hairpin RNA, short hairpin RNA (shRNA), post-transcriptional gene silencing RNA (ptgsRNA), and others.

In certain embodiments, at least one strand of the duplex or double-stranded region of the dsRNA shares sufficient sequence identity or sequence complementarity to the target polynucleotide to allow the dsRNA to reduce the level of expression of the target sequence. In some embodiments, a dsRNA has substantial sequence identity to the target polynucleotide, typically greater than about 65% sequence identity, greater than about 85% sequence identity, about 90%, 91%, 92%, 93%, 94%, 95%, 96%, 97%, 98% or 99% sequence identity. Furthermore, a dsRNA element can be complementary to a portion of the target polynucleotide. Generally, sequences of at least 15, 16, 17, 18, 19, 20, 21, 22, 23, 24, 25, 50, 100, 200, 300, 400, 450 nucleotides or greater of the sequence may be used. As used herein, the strand that is complementary to the target polynucleotide is the “antisense strand” and the strand homologous to the target polynucleotide is the “sense strand.”

In another embodiment, the dsRNA comprises a hairpin RNA. A hairpin RNA comprises an RNA molecule that is capable of folding back onto itself to form a double stranded structure. Multiple structures can be employed as hairpin elements. In certain embodiments, the dsRNA suppression element comprises a hairpin element which comprises in the following order, a first segment, a second segment, and a third segment, where the first and the third segment share sufficient complementarity to allow the transcribed RNA to form a double-stranded stem-loop structure.

The “second segment” of the hairpin comprises a “loop” or a “loop region.” These terms are used synonymously herein and are to be construed broadly to comprise any nucleotide sequence that confers enough flexibility to allow self-pairing to occur between complementary regions of a polynucleotide (i.e., segments 1 and 3 which form the stem of the hairpin). For example, in some embodiments, the loop region may be substantially single stranded and act as a spacer between the self-complementary regions of the hairpin stem-loop. In some embodiments, the loop region can comprise a random or nonsense nucleotide sequence and thus not share sequence identity to a target polynucleotide. In other embodiments, the loop region comprises a sense or an antisense RNA sequence or fragment thereof that shares identity to a target polynucleotide. See, for example, International Patent Publication No. WO 02/00904. In certain embodiments, the loop sequence can include an intron sequence, a sequence derived from an intron sequence, a sequence homologous to an intron sequence, or a modified intron sequence. The intron sequence can be one found in the same or a different species from which segments 1 and 3 are derived. In certain embodiments, the loop region can be optimized to be as short as possible while still providing enough intramolecular flexibility to allow the formation of the base-paired stem region. Accordingly, the loop sequence is generally less than 1000, 900, 800, 700, 600, 500, 400, 300, 200, 100, 50, 25, 20, 19, 18, 17, 16, 15, 10 nucleotides or less.

The “first” and the “third” segment of the hairpin RNA molecule comprise the base-paired stem of the hairpin structure. The first and the third segments are inverted repeats of one another and share sufficient complementarity to allow the formation of the base-paired stem region. In certain embodiments, the first and the third segments are fully complementary to one another. Alternatively, the first and the third segment may be partially complementary to each other so long as they are capable of hybridizing to one another to form a base-paired stem region. The amount of complementarity between the first and the third segment can be calculated as a percentage of the entire segment. Thus, the first and the third segment of the hairpin RNA generally share at least 50%, 60%, 70%, 80%, 85%, 90%, 91%, 92%, 93%, 94%, 95%, 96%, 97%, 98%, 99%, up to and including 100% complementarity.

The first and the third segment are at least about 1000, 500, 475, 450, 425, 400, 375, 350, 325, 300, 250, 225, 200, 175, 150, 125, 100, 75, 60, 50, 40, 30, 25, 22, 21, 20, 19, 18, 17, 16, 15 or 10 nucleotides in length. In certain embodiments, the length of the first and/or the third segment is about 10-100 nucleotides, about 10 to about 75 nucleotides, about 10 to about 50 nucleotides, about 10 to about 40 nucleotides, about 10 to about 35 nucleotides, about 10 to about 30 nucleotides, about 10 to about 25 nucleotides, about 10 to about 19 nucleotides, about 10 to about 20 nucleotides, about 19 to about 50 nucleotides, about 50 nucleotides to about 100 nucleotides, about 100 nucleotides to about 150 nucleotides, about 100 nucleotides to about 300 nucleotides, about 150 nucleotides to about 200 nucleotides, about 200 nucleotides to about 250 nucleotides, about 250 nucleotides to about 300 nucleotides, about 300 nucleotides to about 350 nucleotides, about 350 nucleotides to about 400 nucleotides, about 400 nucleotide to about 500 nucleotides, about 600 nt, about 700 nt, about 800 nt, about 900 nt, about 1000 nt, about 1100 nt, about 1200 nt, 1300 nt, 1400 nt, 1500 nt, 1600 nt, 1700 nt, 1800 nt, 1900 nt, 2000 nt or longer. In other embodiments, the length of the first and/or the third segment comprises at least 10-19 nucleotides, 10-20 nucleotides; 19-35 nucleotides, 20-35 nucleotides; 30-45 nucleotides; 40-50 nucleotides; 50-100 nucleotides; 100-300 nucleotides; about 500-700 nucleotides; about 700-900 nucleotides; about 900-1100 nucleotides; about 1300-1500 nucleotides; about 1500-1700 nucleotides; about 1700-1900 nucleotides; about 1900-2100 nucleotides; about 2100-2300 nucleotides; or about 2300-2500 nucleotides. See, for example, International Publication No. WO 02/00904.

The disclosed hairpin molecules or double-stranded RNA molecules may have more than one disclosed sequence or active fragments or variants, or complements thereof, found in the same portion of the RNA molecule. For example, in a chimeric hairpin structure, the first segment of a hairpin molecule comprises two polynucleotide sections, each with a different disclosed sequence. For example, reading from one terminus of the hairpin, the first segment is composed of sequences from two separate genes (A followed by B). This first segment is followed by the second segment, the loop portion of the hairpin. The loop segment is followed by the third segment, where the complementary strands of the sequences in the first segment are found (B* followed by A*) in forming the stem-loop, hairpin structure, the stem contains SeqA-A* at the distal end of the stem and SeqB-B* proximal to the loop region.

In certain embodiments, the first and the third segment comprise at least 20 nucleotides having at least 85% complementary to the first segment. In still other embodiments, the first and the third segments which form the stem-loop structure of the hairpin comprise 3′ or 5′ overhang regions having unpaired nucleotide residues.

In certain embodiments, the sequences used in the first, the second, and/or the third segments comprise domains that are designed to have sufficient sequence identity to a target polynucleotide of interest and thereby have the ability to decrease the level of expression of the target polynucleotide. The specificity of the inhibitory RNA transcripts is therefore generally conferred by these domains of the silencing element. Thus, in some embodiments, the first, second and/or third segment of the silencing element comprise a domain having at least 10, at least 15, at least 19, at least 20, at least 21, at least 22, at least 23, at least 24, at least 25, at least 30, at least 40, at least 50, at least 100, at least 200, at least 300, at least 500, at least 1000, or more than 1000 nucleotides that share sufficient sequence identity to the target polynucleotide to allow for a decrease in expression levels of the target polynucleotide when expressed in an appropriate cell. In other embodiments, the domain is between about 15 to 50 nucleotides, about 19-35 nucleotides, about 20-35 nucleotides, about 25-50 nucleotides, about 19 to 75 nucleotides, about 20 to 75 nucleotides, about 40-90 nucleotides about 15-100 nucleotides, 10-100 nucleotides, about 10 to about 75 nucleotides, about 10 to about 50 nucleotides, about 10 to about 40 nucleotides, about 10 to about 35 nucleotides, about 10 to about 30 nucleotides, about 10 to about 25 nucleotides, about 10 to about 20 nucleotides, about 10 to about 19 nucleotides, about 50 nucleotides to about 100 nucleotides, about 100 nucleotides to about 150 nucleotides, about 150 nucleotides to about 200 nucleotides, about 200 nucleotides to about 250 nucleotides, about 250 nucleotides to about 300 nucleotides, about 300 nucleotides to about 350 nucleotides, about 350 nucleotides to about 400 nucleotides, about 400 nucleotide to about 500 nucleotides or longer. In other embodiments, the length of the first and/or the third segment comprises at least 10-20 nucleotides, at least 10-19 nucleotides, 20-35 nucleotides, 30-45 nucleotides, 40-50 nucleotides, 50-100 nucleotides, or about 100-300 nucleotides.

In certain embodiments, a domain of the first, the second, and/or the third segment has 100% sequence identity to the target polynucleotide. In other embodiments, the domain of the first, the second and/or the third segment having homology to the target polynucleotide have at least 50%, 60%, 70%, 80%, 85%, 90%, 91%, 92%, 93%, 94%, 95%, 96%, 97%, 98%, 99%, or greater sequence identity to a region of the target polynucleotide. The sequence identity of the domains of the first, the second and/or the third segments complementary to a target polynucleotide need only be sufficient to decrease expression of the target polynucleotide of interest. See, for example, Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. Patent Publication No. 20030175965. A transient assay for the efficiency of hpRNA constructs to silence gene expression in vivo has been described by Panstruga et al. (2003)Mol. Biol. Rep. 30:135-140.

The amount of complementarity shared between the first, second, and/or third segment and the target polynucleotide or the amount of complementarity shared between the first segment and the third segment (i.e., the stem of the hairpin structure) may vary depending on the organism in which gene expression is to be controlled. Some organisms or cell types may require exact pairing or 100% identity, while other organisms or cell types may tolerate some mismatching. In some cells, for example, a single nucleotide mismatch in the targeting sequence abrogates the ability to suppress gene expression. In these cells, the disclosed suppression cassettes can be used to target the suppression of mutant genes, for example, oncogenes whose transcripts comprise point mutations and therefore they can be specifically targeted using the methods and compositions disclosed herein without altering the expression of the remaining wild-type allele. In other organisms, holistic sequence variability may be tolerated as long as some 22 nt region of the sequence is represented in 100% homology between target polynucleotide and the suppression cassette.

Any region of the target polynucleotide can be used to design a domain of the silencing element that shares sufficient sequence identity to allow expression of the hairpin transcript to decrease the level of the target polynucleotide. For instance, a domain may be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof. In certain embodiments, a domain of the silencing element shares sufficient identity, homology, or is complementary to at least about 15, 16, 17, 18, 19, 20, 22, 25 or 30 consecutive nucleotides from about nucleotides 1-50, 25-75, 75-125, 50-100, 125-175, 175-225, 100-150, 150-200, 200-250, 225-275, 275-325, 250-300, 325-375, 375-425, 300-350, 350-400, 425-475, 400-450, 475-525, 450-500, 525-575, 575-625, 550-600, 625-675, 675-725, 600-650, 625-675, 675-725, 650-700, 725-825, 825-875, 750-800, 875-925, 925-975, 850-900, 925-975, 975-1025, 950-1000, 1000-1050, 1025-1075, 1075-1125, 1050-1100, 1125-1175, 1100-1200, 1175-1225, 1225-1275, 1200-1300, 1325-1375, 1375-1425, 1300-1400, 1425-1475, 1475-1525, 1400-1500, 1525-1575, 1575-1625, 1625-1675, 1675-1725, 1725-1775, 1775-1825, 1825-1875, 1875-1925, 1925-1975, 1975-2025, 2025-2075, 2075-2125, 2125-2175, 2175-2225, 1500-1600, 1600-1700, 1700-1800, 1800-1900, 1900-2000 of the target sequence. In some instances, to optimize the siRNA sequences employed in the hairpin, the synthetic oligodeoxyribonucleotide/RNAse H method can be used to determine sites on the target mRNA that are in a conformation that is susceptible to RNA silencing. See, for example, Vickers et al. (2003) J. Biol. Chem 278:7108-7118 and Yang et al. (2002) Proc. Natl. Acad. Sci. USA 99:9442-9447. These studies indicate that there is a significant correlation between the RNase-H-sensitive sites and sites that promote efficient siRNA-directed mRNA degradation.

The hairpin silencing element may also be designed such that the sense sequence or the antisense sequence do not correspond to a target polynucleotide. In this embodiment, the sense and antisense sequence flank a loop sequence that comprises a nucleotide sequence corresponding to all or part of the target polynucleotide. Thus, it is the loop region that determines the specificity of the RNA interference. See, for example, WO 02/00904.

In addition, transcriptional gene silencing (TGS) may be accomplished through use of a hairpin suppression element where the inverted repeat of the hairpin shares sequence identity with the promoter region of a target polynucleotide to be silenced. See, for example, Aufsatz et al. (2002) PNAS 99 (Suppl. 4): 16499-16506 and Mette et al. (2000) EMBO J 19(19):5194-5201.

In other embodiments, the silencing element can comprise a small RNA (sRNA). sRNAs can comprise both micro RNA (miRNA) and short-interfering RNA (siRNA) (Meister and Tuschl (2004) Nature 431:343-349 and Bonetta et al. (2004) Nature Methods 1:79-86). miRNAs are regulatory agents comprising about 19 to about 24 ribonucleotides in length which are highly efficient at inhibiting the expression of target polynucleotides. See, for example Javier et al. (2003) Nature 425: 257-263. For miRNA interference, the silencing element can be designed to express a dsRNA molecule that forms a hairpin structure or partially base-paired structure containing a 19, 20, 21, 22, 23, 24 or 25 nucleotide sequence that is complementary to the target polynucleotide of interest. The miRNA can be synthetically made, or transcribed as a longer RNA which is subsequently cleaved to produce the active miRNA. Specifically, the miRNA can comprise 19 nucleotides of the sequence having homology to a target polynucleotide in sense orientation and 19 nucleotides of a corresponding antisense sequence that is complementary to the sense sequence. The miRNA can be an “artificial miRNA” or “amiRNA” which comprises a miRNA sequence that is synthetically designed to silence a target sequence.

When expressing a miRNA the final (mature) miRNA is present in a duplex in a precursor backbone structure, the two strands being referred to as the miRNA (the strand that will eventually base pair with the target) and miRNA*(star sequence). It has been demonstrated that miRNAs can be transgenically expressed and target genes of interest for efficient silencing (Highly specific gene silencing by artificial microRNAs in Arabidopsis Schwab R, Ossowski S, Riester M, Warthmann N, Weigel D. Plant Cell. 2006 May; 18(5):1121-33. Epub 2006 Mar. 10; and Expression of artificial microRNAs in transgenic Arabidopsis thaliana confers virus resistance. Niu Q W, Lin S S, Reyes J L, Chen K C, Wu H W, Yeh S D, Chua N H. Nat Biotechnol. 2006 November; 24(11): 1420-8. Epub 2006 Oct. 22. Erratum in: Nat Biotechnol. 2007 February; 25(2):254.).

The silencing element for miRNA interference comprises a miRNA primary sequence. The miRNA primary sequence comprises a DNA sequence having the miRNA and star sequences separated by a loop as well as additional sequences flanking this region that are important for processing. When expressed as an RNA, the structure of the primary miRNA is such as to allow for the formation of a hairpin RNA structure that can be processed into a mature miRNA. In some embodiments, the miRNA backbone comprises a genomic or cDNA miRNA precursor sequence, wherein said sequence comprises a native primary in which a heterologous (artificial) mature miRNA and star sequence are inserted.

As used herein, a “star sequence” is the sequence within a miRNA precursor backbone that is complementary to the miRNA and forms a duplex with the miRNA to form the stem structure of a hairpin RNA. In some embodiments, the star sequence can comprise less than 100% complementarity to the miRNA sequence. Alternatively, the star sequence can comprise at least 99%, 98%, 97%, 96%, 95%, 90%, 85%, 80% or lower sequence complementarity to the miRNA sequence as long as the star sequence has sufficient complementarity to the miRNA sequence to form a double stranded structure. In still further embodiments, the star sequence comprises a sequence having 1, 2, 3, 4, 5 or more mismatches with the miRNA sequence and still has sufficient complementarity to form a double stranded structure with the miRNA sequence resulting in the production of miRNA and suppression of the target sequence.

The miRNA precursor backbones can be from any plant. In some embodiments, the miRNA precursor backbone is from a monocot. In other embodiments, the miRNA precursor backbone is from a dicot. In further embodiments, the backbone is from maize or soybean. MicroRNA precursor backbones have been described previously. For example, US20090155910A1 (WO 2009/079532) discloses the following soybean miRNA precursor backbones: 156c, 159, 166b, 168c, 396b and 398b, and US20090155909A1 (WO 2009/079548) discloses the following maize miRNA precursor backbones: 159c, 164h, 168a, 169r, and 396h.

Thus, the primary miRNA can be altered to allow for efficient insertion of heterologous miRNA and star sequences within the miRNA precursor backbone. In such instances, the miRNA segment and the star segment of the miRNA precursor backbone are replaced with the heterologous miRNA and the heterologous star sequences, designed to target any sequence of interest, using a PCR technique and cloned into an expression construct. It is recognized that there could be alterations to the position at which the artificial miRNA and star sequences are inserted into the backbone. Detailed methods for inserting the miRNA and star sequence into the miRNA precursor backbone are described in, for example, US Patent Applications 20090155909A1 and US20090155910A1.

When designing a miRNA sequence and star sequence, various design choices can be made. See, for example, Schwab R, et al. (2005) Dev Cell 8: 517-27. In non-limiting embodiments, the miRNA sequences disclosed herein can have a “U” at the 5′-end, a “C” or “G” at the 19th nucleotide position, and an “A” or “U” at the 10th nucleotide position. In other embodiments, the miRNA design is such that the miRNA have a high free delta-G as calculated using the ZipFold algorithm (Markham, N. R. & Zuker, M. (2005) Nucleic Acids Res. 33: W577-W581.) Optionally, a one base pair change can be added within the 5′ portion of the miRNA so that the sequence differs from the target sequence by one nucleotide.

The methods and compositions disclosed herein employ DNA constructs that when transcribed “form” a silencing element, such as a dsRNA molecule. The methods and compositions also may comprise a host cell comprising the DNA construct encoding a silencing element. In another embodiment, the methods and compositions also may comprise a transgenic plant comprising the DNA construct encoding a silencing element. Accordingly, the heterologous polynucleotide being expressed need not form the dsRNA by itself, but can interact with other sequences in the plant cell or in the pest gut after ingestion to allow the formation of the dsRNA. For example, a chimeric polynucleotide that can selectively silence the target polynucleotide can be generated by expressing a chimeric construct comprising the target sequence for a miRNA or siRNA to a sequence corresponding to all or part of the gene or genes to be silenced. In this embodiment, the dsRNA is “formed” when the target for the miRNA or siRNA interacts with the miRNA present in the cell. The resulting dsRNA can then reduce the level of expression of the gene or genes to be silenced. See, for example, US Application Publication 2007-0130653, entitled “Methods and Compositions for Gene Silencing”. The construct can be designed to have a target for an endogenous miRNA or alternatively, a target for a heterologous and/or synthetic miRNA can be employed in the construct. If a heterologous and/or synthetic miRNA is employed, it can be introduced into the cell on the same nucleotide construct as the chimeric polynucleotide or on a separate construct. As discussed elsewhere herein, any method can be used to introduce the construct comprising the heterologous miRNA.

### IV. Variants and Fragments

By “fragment” is intended a portion of the polynucleotide or a portion of the amino acid sequence and hence protein encoded thereby. Fragments of a polynucleotide may encode protein fragments that retain the biological activity of the native protein. Alternatively, fragments of a polynucleotide that are useful as a silencing element do not need to encode fragment proteins that retain biological activity. Thus, fragments of a nucleotide sequence may range from at least about 10, about 15, about 16, about 17, about 18, about 19, nucleotides, about 20 nucleotides, about 21 nucleotides, about 22 nucleotides, about 50 nucleotides, about 75 nucleotides, about 100 nucleotides, 200 nucleotides, 300 nucleotides, 400 nucleotides, 500 nucleotides, 600 nucleotides, 700 nucleotides and up to and including one nucleotide less than the full-length polynucleotide employed. Alternatively, fragments of a nucleotide sequence may range from 1-50, 25-75, 75-125, 50-100, 125-175, 175-225, 100-150, 100-300, 150-200, 200-250, 225-275, 275-325, 250-300, 325-375, 375-425, 300-350, 350-400, 425-475, 400-450, 475-525, 450-500, 525-575, 575-625, 550-600, 625-675, 675-725, 600-650, 625-675, 675-725, 650-700, 725-825, 825-875, 750-800, 875-925, 925-975, 850-900, 925-975, 975-1025, 950-1000, 1000-1050, 1025-1075, 1075-1125, 1050-1100, 1125-1175, 1100-1200, 1175-1225, 1225-1275, 1200-1300, 1325-1375, 1375-1425, 1300-1400, 1425-1475, 1475-1525, 1400-1500, 1525-1575, 1575-1625, 1625-1675, 1675-1725, 1725-1775, 1775-1825, 1825-1875, 1875-1925, 1925-1975, 1975-2025, 2025-2075, 2075-2125, 2125-2175, 2175-2225, 1500-1600, 1600-1700, 1700-1800, 1800-1900, 1900-2000. Methods to assay for the activity of a desired silencing element are described elsewhere herein.

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. A variant of a polynucleotide that is useful as a silencing element will retain the ability to reduce expression of the target polynucleotide and, in some embodiments, thereby control a plant insect pest of interest. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the disclosed polypeptides. Variant polynucleotides also include synthetically derived polynucleotide, such as those generated, for example, by using site-directed mutagenesis, but continue to retain the desired activity. Generally, variants of a particular disclosed polynucleotide (i.e., a silencing element) will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular disclosed polynucleotide (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of disclosed polynucleotides employed is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. To determine the percent identity of two amino acid sequences or of two nucleic acid sequences, the sequences are aligned for optimal comparison purposes. The percent identity between the two sequences is a function of the number of identical positions shared by the sequences (e.g i.e., percent identity of query sequence=number of identical positions between query and subject sequences/total number of positions of query sequence (e.g., overlapping positions)×100).

A method is further provided for identifying a silencing element. Such methods comprise obtaining a candidate fragment, which is of sufficient length to act as a silencing element and thereby reduce the expression of the target polynucleotide and/or control a desired pest; expressing said candidate silencing element and a polynucleotide encoding a MWLMV or JCSMV virus or modified MWLMV or JCSMV virus, such as the sequences set forth in SEQ ID NOS.: 1-22, or variants and fragments thereof, in an appropriate expression cassette to produce the candidate silencing element and a MWLMV or JCSMV virus or modified MWLMV or JCSMV virus, and determining if said candidate polynucleotide fragment has the activity of a silencing element and thereby reduce the expression of the target polynucleotide and/or controls a desired pest. Further, the method may comprise comparing the candidate to a silencing element known to reduce the expression of the target polynucleotide and/or controls a desired pest. Methods of identifying such candidate fragments based on the desired pathway for suppression are known. For example, various bioinformatics programs can be employed to identify the region of the target polynucleotides that could be exploited to generate a silencing element. See, for example, Elbahir et al. (2001) Genes and Development 15:188-200, Schwartz et al. (2003) Cell 115:199-208, Khvorova et al. (2003) Cell 115:209-216. See also, siRNA at Whitehead (jura.wi.mit.edu/bioc/siRNAext/) which calculates the binding energies for both sense and antisense siRNAs. See also, genscript.com/ssl-bin/app/rnai?op=known; Block-iT™ RNAi designer from Invitrogen and GenScript siRNA Construct Builder.

### V. DNA Constructs

The use of the term “polynucleotide” is not intended to be limiting to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The disclosed polynucleotides also encompass all forms of sequences including, but not limited to, single-stranded forms, double-stranded forms, hairpins, stem-and-loop structures, and the like.

The polynucleotide encoding the silencing element and a polynucleotide encoding a MWLMV or JCSMV virus or modified MWLMV or JCSMV virus, or in specific embodiments, employed in the disclosed methods and compositions can be provided in expression cassettes for expression in a plant or organism of interest. In one embodiment, a DNA construct comprises a polynucleotide encoding a silencing element and a a polynucleotide encoding a MWLMV virus, modified MWLMV virus, and MWLMV satellite, a MWLMV coat polypeptide, a MWLMV suppressor of RNA silencing, a satellite MWLMV coat polypeptide, a movement polypeptide, and/or a RNA-directed RNA polymerase polypeptide. In some embodiments, a DNA construct comprises a a polynucleotide encoding silencing element and a polynucleotide encoding a MWLMV virus as set forth in SEQ ID NOS: 1-29.

In another embodiment, the a silencing element may be expressed from a first DNA construct, and a polynucleotide encoding a MWLMV or JCSMV virus or modified MWLMV or JCSMV virus, such as SEQ ID NOs: 1-22, may be expressed in a second DNA construct. These two constructs may be transformed and expressed in one host cell or transformed and expressed in separate host cells. It is recognized that multiple silencing elements including multiple identical silencing elements, multiple silencing elements targeting different regions of the target sequence, or multiple silencing elements from different target sequences can be used. In this embodiment, it is recognized that each polynucleotide encoding silencing element and each polynucleotide encoding a MWLMV or JCSMV virus or modified MWLMV or JCSMV virus may be contained in a single or separate cassette, DNA construct, or vector. As discussed, any means of providing the silencing element and a MWLMV or JCSMV virus or modified MWLMV or JCSMV virus is contemplated. A plant or plant cell can be transformed with a single cassette comprising DNA encoding one or more silencing elements and one or more MWLMV or JCSMV viruses or modified MWLMV or JCSMV viruses or separate cassettes comprising each polynucleotide encoding silencing element and each polynucleotide encoding a MWLMV or JCSMV virus or modified MWLMV or JCSMV virus may be used to transform a plant or plant cell, bacterial cell, or host cell. Likewise, a plant transformed with one component can be subsequently transformed with the second component. One or more polynucleotides encoding silencing elements and one or more polynucleotides encoding MWLMV or JCSMV viruses or modified MWLMV or JCSMV viruses can also be brought together by sexual crossing. That is, a first plant comprising one component is crossed with a second plant comprising the second component. Progeny plants from the cross will comprise both components.

The expression cassette can include 5′ and 3′ regulatory sequences operably linked to the polynucleotide of the invention. “Operably linked” is intended to mean a functional linkage between two or more elements. For example, an operable linkage between a polynucleotide of the invention and a regulatory sequence (i.e., a promoter) is a functional link that allows for expression of the polynucleotide of the invention. Operably linked elements may be contiguous or non-contiguous. When used to refer to the joining of two protein coding regions, by operably linked is intended that the coding regions are in the same reading frame. The cassette may additionally contain at least one additional polynucleotide to be cotransformed into the organism. Alternatively, the additional polypeptide(s) can be provided on multiple expression cassettes. Expression cassettes can be provided with a plurality of restriction sites and/or recombination sites for insertion of the polynucleotide to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a polynucleotide encoding the silencing element and a polynucleotide encoding a MWLMV or JCSMV virus or modified MWLMV or JCSMV virus, which may include a MWLMV, modified MWLMV, and MWLMV satellite, a MWLMV coat polypeptide, a MWLMV suppressor of RNA silencing, a satellite MWLMV coat polypeptide, a movement polypeptide, and/or a RNA-directed RNA polymerase polypeptide, employed in the methods and compositions of provided herein, and a transcriptional and translational termination region (i.e., termination region) functional in plants. In another embodiment, the double stranded RNA and the MWLMV or JCSMV virus or modified MWLMV or JCSMV virus are expressed from a suppression cassette. Such a cassette may comprise two convergent promoters that drive transcription of an operably linked silencing element. “Convergent promoters” refers to promoters that are oriented on either terminus of the operably linked silencing element such that each promoter drives transcription of the silencing element in opposite directions, yielding two transcripts. In such embodiments, the convergent promoters allow for the transcription of the sense and anti-sense strand and thus allow for the formation of a dsRNA. Such a cassette may also comprise two divergent promoters that drive transcription of one or more operably linked silencing elements. “Divergent promoters” refers to promoters that are oriented in opposite directions of each other, driving transcription of the one or more silencing elements in opposite directions. In such embodiments, the divergent promoters allow for the transcription of the sense and antisense strands and allow for the formation of a dsRNA. In such embodiments, the divergent promoters also allow for the transcription of at least two separate hairpin RNAs. In another embodiment, one cassette comprising two or more silencing elements under the control of two separate promoters in the same orientation is present in a construct. In another embodiment, two or more individual cassettes, each comprising at least one silencing element under the control of a promoter, are present in a construct in the same orientation.

The regulatory regions (i.e., promoters, transcriptional regulatory regions, and translational termination regions) and/or the polynucleotides employed in the invention may be native/analogous to the host cell or to each other. Alternatively, the regulatory regions and/or the polynucleotide employed in the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

The termination region may be native with the transcriptional initiation region, may be native with the operably linked polynucleotide encoding the silencing element and MWLMV or JCSMV virus or modified MWLMV or JCSMV virus, may be native with the plant host, or may be derived from another source (i.e., foreign or heterologous) to the promoter, the polynucleotide comprising silencing element, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the present embodiments. The polynucleotide encoding the silencing element can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

An inducible promoter, for instance, a pathogen-inducible promoter could also be employed. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) PlantMol. Biol. 22:783-792; Eckelkamp et al. (1993) FEBS Letters 323:73-76); MPI gene (Corderok et al. (1994) Plant J. 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where the application of the chemical induces gene expression, or a chemical-repressible promoter, where the application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991)Mol. Gen. Genet. 227:229-237, and U.S. Pat. Nos. 5,814,618 and 5,789,156), herein incorporated by reference.

Tissue-preferred promoters can be utilized to target enhanced expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) TransgenicRes. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6): 1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6): 1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1): 11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptll (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

In an embodiment, the plant-expressed promoter is a vascular-specific promoter such as a phloem-specific promoter. A “vascular-specific” promoter, as used herein, is a promoter which is at least expressed in vascular cells, or a promoter which is preferentially expressed in vascular cells. Expression of a vascular-specific promoter need not be exclusively in vascular cells, expression in other cell types or tissues is possible. A “phloem-specific promoter” as used herein, is a plant-expressible promoter which is at least expressed in phloem cells, or a promoter which is preferentially expressed in phloem cells.

Expression of a phloem-specific promoter need not be exclusively in phloem cells, expression in other cell types or tissues, e.g., xylem tissue, is possible. In one embodiment of this invention, a phloem-specific promoter is a plant-expressible promoter at least expressed in phloem cells, wherein the expression in non-phloem cells is more limited (or absent) compared to the expression in phloem cells. Examples of suitable vascular-specific or phloem-specific promoters in accordance with this invention include but are not limited to the promoters selected from the group consisting of: the SCSV3, SCSV4, SCSV5, and SCSV7 promoters (Schunmann et al. (2003) Plant Functional Biology 30:453-60; the rolC gene promoter of Agrobacterium rhizogenes(Kiyokawa et al. (1994) Plant Physiology 104:801-02; Pandolfini et al. (2003) BioMedCentral (BMC) Biotechnology 3:7, (www.biomedcentral.com/1472-6750/3/7); Graham et al. (1997) Plant Mol. Biol. 33:729-35; Guivarc'h et al. (1996); Almon et al. (1997) Plant Physiol. 115:1599-607; the rolA gene promoter of Agrobacterium rhizogenes (Dehio et al. (1993) Plant Mol. Biol. 23:1199-210); the promoter of the Agrobacterium tumefaciens T-DNA gene 5 (Korber et al. (1991) EMBO J. 10:3983-91); the rice sucrose synthase RSsl gene promoter (Shi et al. (1994) J. Exp. Bot. 45:623-31); the CoYMV or Commelina yellow mottle badnavirus promoter (Medberry et al. (1992) Plant Cell 4:185-92; Zhou et al. (1998) Chin. J. Biotechnol. 14:9-16); the CFDV or coconut foliar decay virus promoter (Rohde et al. (1994) Plant Mol. Biol. 27:623-28; Hehn and Rhode (1998) J. Gen. Virol. 79:1495-99); the RTBV or rice tungro bacilliform virus promoter (Yin and Beachy (1995) Plant J. 7:969-80; Yin et al. (1997) Plant J. 12:1179-80); the pea glutamin synthase GS3A gene (Edwards et al. (1990) Proc. Natl. Acad. Sci. USA 87:3459-63; Brears et al. (1991) Plant J. 1:235-44); the inv CD111 and inv CD141 promoters of the potato invertase genes (Hedley et al. (2000) J. Exp. Botany 51:817-21); the promoter isolated from Arabidopsis shown to have phloem-specific expression in tobacco by Kertbundit et al. (1991) Proc. Natl. Acad. Sci. USA 88:5212-16); the VAHOX1 promoter region (Tornero et al. (1996) Plant J. 9:639-48); the pea cell wall invertase gene promoter (Zhang et al. (1996) Plant Physiol. 112:1111-17); the promoter of the endogenous cotton protein related to chitinase of US published patent application 20030106097, an acid invertase gene promoter from carrot (Ramloch-Lorenz et al. (1993) The Plant J. 4:545-54); the promoter of the sulfate transporter geneSultrl; 3 (Yoshimoto et al. (2003) Plant Physiol. 131:1511-17); a promoter of a sucrose synthase gene (Nolte and Koch (1993) Plant Physiol. 101:899-905); and the promoter of a tobacco sucrose transporter gene (Kuhn et al. (1997) Science 275-1298-1300).

Possible promoters also include the Black Cherry promoter for Prunasin Hydrolase (PH DL1.4 PRO) (U.S. Pat. No. 6,797,859), Thioredoxin H promoter from cucumber and rice (Fukuda A et al. (2005). Plant Cell Physiol. 46(11): 1779-86), Rice (RSsl) (Shi, T. Wang et al. (1994). J. Exp. Bot. 45(274): 623-631) and maize sucrose synthese-1 promoters (Yang., N-S. et al. (1990) PNAS 87:4144-4148), PP2 promoter from pumpkin Guo, H. et al. (2004) Transgenic Research 13:559-566), At SUC2 promoter (Truernit, E. et al. (1995) Planta 196(3):564-70., At SAM-1 (S-adenosylmethionine synthetase) (Mijnsbrugge K V. et al. (1996) Planr. Cell. Physiol. 37(8): 1108-1115), and the Rice tungro bacilliform virus (RTBV) promoter (Bhattacharyya-Pakrasi et al. (1993) Plant J. 4(1):71-79).

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as 3-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004)J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990)Mol. Cell. Biol. 10:3343-3356; Zambretti et al. (1992)Proc. Natl. Acad Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not meant to be limiting. Any selectable marker gene can be used in the present invention.

### VI. Proteins and Variants and Fragments Thereof

One aspect of the disclosure is MWLMV or JCSMV virus or modified MWLMV or JCSMV virus polypeptides. A MWLMV, modified MWLMV, and MWLMV satellite, a MWLMV coat polypeptide, a MWLMV suppressor of RNA silencing, a satellite MWLMV coat polypeptide, a movement polypeptide, and/or a RNA-directed RNA polymerase polypeptide are encompassed by the disclosure. In some embodiments, a MWLMV or JCSMV virus or modified MWLMV or JCSMV virus or a polypeptide sufficiently homologous to any one of the polypeptides, fragments, or variants of SEQ ID NOs: 117-122 and 140-144 are provided. A variety of MWLMV or JCSMV virus or modified MWLMV or JCSMV virus polypeptides are contemplated. One source of a MWLMV or JCSMV virus or modified MWLMV or JCSMV virus polypeptide or related proteins is a viral strain that contains the polynucleotide of SEQ ID NOs: 1-14 that encode the polypeptides of SEQ ID NOs: 117-122 and 140-144 (See Table 2 and Table 8). In some embodiments a MWLMV or JCSMV virus or modified MWLMV or JCSMV virus polypeptide is sufficiently identical to an amino acid sequence of SEQ ID NOs: 117-122 and 140-144. “Sufficiently identical” is used herein to refer to an amino acid sequence that has at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to a reference sequence using one of the alignment programs described herein using standard parameters. One of skill in the art will recognize that these values can be appropriately adjusted to determine corresponding homology of proteins taking into account amino acid similarity and the like.

In some embodiments a MWLMV or JCSMV virus or modified MWLMV or JCSMV virus polypeptide has at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NOs: 117-122 and 140-144.

As used herein, the terms “protein,” “peptide molecule,” or “polypeptide” includes any molecule that comprises five or more amino acids. It is well known in the art that protein, peptide or polypeptide molecules may undergo modification, including post-translational modifications, such as, but not limited to, disulfide bond formation, glycosylation, phosphorylation or oligomerization. Thus, as used herein, the terms “protein,” “peptide molecule” or “polypeptide” includes any protein that is modified by any biological or non-biological process. The terms “amino acid” and “amino acids” refer to all naturally occurring L-amino acids.

In some embodiments the polypeptides of the disclosure have a modified physical property. As used herein, the term “physical property” refers to any parameter suitable for describing the physical-chemical characteristics of a protein. As used herein, “physical property of interest” and “property of interest” are used interchangeably to refer to physical properties of proteins that are being investigated and/or modified. Examples of physical properties include, but are not limited to net surface charge and charge distribution on the protein surface, net hydrophobicity and hydrophobic residue distribution on the protein surface, surface charge density, surface hydrophobicity density, total count of surface ionizable groups, surface tension, protein size and its distribution in solution, melting temperature, heat capacity, and second virial coefficient. Examples of physical properties also include, but are not limited to solubility, folding, stability, and digestibility. In some embodiments the polypeptides of the disclosure have increased digestibility of proteolytic fragments in an insect gut. Models for digestion by simulated gastric fluids are known to one skilled in the art (Fuchs, R. L. and J. D. Astwood. Food Technology 50: 83-88, 1996; Astwood, J. D., et al Nature Biotechnology 14: 1269-1273, 1996; Fu T J et al J. Agric Food Chem. 50: 7154-7160, 2002).

In some embodiments variants include polypeptides that differ in amino acid sequence due to mutagenesis. Variant proteins encompassed by the disclosure are biologically active, that is they continue to possess the desired biological activity (i.e. pesticidal activity) of the native protein. In some embodiment the variant will have at least about 10%, at least about 30%, at least about 50%, at least about 70%, at least about 80% or more of the activity of the native protein. In some embodiments, the variants may have improved activity over the native protein.

In another aspect fusion proteins are provided that include within its amino acid sequence an amino acid sequence comprising a polypeptide of the disclosure. Methods for design and construction of fusion proteins, and polynucleotides encoding the same, are known to those of skill in the art. Polynucleotides encoding a MWLMV or JCSMV virus or modified MWLMV or JCSMV virus polypeptide of the disclosure may be fused to signal sequences which will direct the localization of the MWLMV or JCSMV virus or modified MWLMV or JCSMV virus polypeptide of the disclosure to a MWLMV or JCSMV virus or modified MWLMV or JCSMV virus polypeptide of the embodiments from a prokaryotic or eukaryotic cell. For example, in E. coli, one may wish to direct the expression of the protein to the periplasmic space. Examples of signal sequences or proteins (or fragments thereof) to which the polypeptide of the disclosure may be fused in order to direct the expression of the polypeptide to the periplasmic space of bacteria include, but are not limited to, the pelB signal sequence, the maltose binding protein (MBP) signal sequence, MBP, the ompA signal sequence, the signal sequence of the periplasmic E. coli heat-labile enterotoxin B-subunit and the signal sequence of alkaline phosphatase. Several vectors are commercially available for the construction of fusion proteins which will direct the localization of a protein, such as the pMAL series of vectors (particularly the pMAL-p series) available from New England Biolabs® (240 County Road, Ipswich, Mass. 01938-2723). In a specific embodiment, the polypeptide of the disclosure may be fused to the pelB pectate lyase signal sequence to increase the efficiency of expression and purification of such polypeptides in Gram-negative bacteria (see, U.S. Pat. Nos. 5,576,195 and 5,846,818). Plant plastid transit peptide/polypeptide fusions are well known in the art (see, U.S. Pat. No. 7,193,133). Apoplast transit peptides such as rice or barley alpha-amylase secretion signal are also well known in the art. The plastid transit peptide is generally fused N-terminal to the polypeptide to be targeted (e.g., the fusion partner). In one embodiment, the fusion protein consists essentially of the plastid transit peptide and the polypeptide of the disclosure to be targeted. In another embodiment, the fusion protein comprises the plastid transit peptide and the polypeptide to be targeted. In such embodiments, the plastid transit peptide is preferably at the N-terminus of the fusion protein. However, additional amino acid residues may be N-terminal to the plastid transit peptide providing that the fusion protein is at least partially targeted to a plastid. In a specific embodiment, the plastid transit peptide is in the N-terminal half, N-terminal third or N-terminal quarter of the fusion protein. Most or all of the plastid transit peptide is generally cleaved from the fusion protein upon insertion into the plastid. The position of cleavage may vary slightly between plant species, at different plant developmental stages, as a result of specific intercellular conditions or the particular combination of transit peptide/fusion partner used. In one embodiment, the plastid transit peptide cleavage is homogenous such that the cleavage site is identical in a population of fusion proteins. In another embodiment, the plastid transit peptide is not homogenous, such that the cleavage site varies by 1-10 amino acids in a population of fusion proteins. The plastid transit peptide can be recombinantly fused to a second protein in one of several ways. For example, a restriction endonuclease recognition site can be introduced into the nucleotide sequence of the transit peptide at a position corresponding to its C-terminal end and the same or a compatible site can be engineered into the nucleotide sequence of the protein to be targeted at its N-terminal end. Care must be taken in designing these sites to ensure that the coding sequences of the transit peptide and the second protein are kept “in frame” to allow the synthesis of the desired fusion protein. In some cases, it may be preferable to remove the initiator methionine codon of the second protein when the new restriction site is introduced. The introduction of restriction endonuclease recognition sites on both parent molecules and their subsequent joining through recombinant DNA techniques may result in the addition of one or more extra amino acids between the transit peptide and the second protein. This generally does not affect targeting activity as long as the transit peptide cleavage site remains accessible and the function of the second protein is not altered by the addition of these extra amino acids at its N-terminus. Alternatively, one skilled in the art can create a precise cleavage site between the transit peptide and the second protein (with or without its initiator methionine) using gene synthesis (Stemmer, et al., (1995) Gene 164:49-53) or similar methods. In addition, the transit peptide fusion can intentionally include amino acids downstream of the cleavage site. The amino acids at the N-terminus of the mature protein can affect the ability of the transit peptide to target proteins to plastids and/or the efficiency of cleavage following protein import. This may be dependent on the protein to be targeted. See, e.g., Comai, et al., (1988) J. Biol. Chem. 263(29):15104-9.

In another aspect chimeric MWLMV or JCSMV virus or modified MWLMV or JCSMV virus polypeptides are provided that are created by joining two or more portions of MWLMV or JCSMV virus or modified MWLMV or JCSMV virus polypeptide genes of disclosure, which originally encoded separate MWLMV or JCSMV virus or modified MWLMV or JCSMV virus proteins to create a chimeric gene. The translation of the chimeric gene results in a single chimeric polypeptide with regions, motifs or domains derived from each of the original polypeptides.

It is recognized that DNA sequences may be altered by various methods, and that these alterations may result in DNA sequences encoding proteins with amino acid sequences different than that encoded by the wild-type (or native) protein. In some embodiments a polypeptide of the disclosure may be altered in various ways including amino acid substitutions, deletions, truncations and insertions of one or more amino acids, including up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or more amino acid substitutions, deletions and/or insertions or combinations thereof compared to any one of SEQ ID NOs: 117-122 and 140-144. In some embodiments a polypeptide of the disclosure comprises the deletion of 1, 2, 3, 4, 5, 6, 7, 8, 9, 10, 11, 12, 13, 14, 15 or more amino acids from the N-terminus and/or C-terminus of the polypeptide of the disclosure.

Methods for such manipulations are generally known in the art. For example, amino acid sequence variants of an polypeptide of the disclosure can be prepared by mutations in the DNA. This may also be accomplished by one of several forms of mutagenesis and/or in directed evolution. In some aspects, the changes encoded in the amino acid sequence will not substantially affect the function of the protein. Such variants will possess the desired activity. However, it is understood that the ability of a polypeptide of the disclosure to confer activity may be improved by the use of such techniques upon the compositions of this disclosure.

For example, conservative amino acid substitutions may be made at one or more, predicted, nonessential amino acid residues. A “nonessential” amino acid residue is a residue that can be altered from the wild-type sequence of an polypeptide of the disclosure without altering the biological activity. A “conservative amino acid substitution” is one in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); polar, negatively charged residues and their amides (e.g., aspartic acid, asparagine, glutamic acid, glutamine; uncharged polar side chains (e.g., glycine, asparagine, glutamine, serine, threonine, tyrosine, cysteine); small aliphatic, nonpolar or slightly polar residues (e.g., Alanine, serine, threonine, proline, glycine); nonpolar side chains (e.g., alanine, valine, leucine, isoleucine, proline, phenylalanine, methionine, tryptophan); large aliphatic, nonpolar residues (e.g., methionine, leucine, isoleucine, valine, cysteine); beta-branched side chains (e.g., threonine, valine, isoleucine); aromatic side chains (e.g., tyrosine, phenylalanine, tryptophan, histidine); large aromatic side chains (e.g., tyrosine, phenylalanine, tryptophan).

Amino acid substitutions may be made in nonconserved regions that retain function. In general, such substitutions would not be made for conserved amino acid residues or for amino acid residues residing within a conserved motif, where such residues are essential for protein activity. Examples of residues that are conserved and that may be essential for protein activity include, for example, residues that are identical between all proteins contained in an alignment of similar or related toxins to the sequences of the embodiments (e.g., residues that are identical in an alignment of homologs). Examples of residues that are conserved but that may allow conservative amino acid substitutions and still retain activity include, for example, residues that have only conservative substitutions between all proteins contained in an alignment of similar or related MWLMV or JCSMV viruses or modified MWLMV or JCSMV viruses to the sequences of the embodiments (e.g., residues that have only conservative substitutions between all proteins contained in the alignment of the homologs). However, one of skill in the art would understand that functional variants may have minor conserved or nonconserved alterations in the conserved residues. Guidance as to appropriate amino acid substitutions that do not affect biological activity of the protein of interest may be found in the model of Dayhoff, et al., (1978) Atlas of Protein Sequence and Structure (Natl. Biomed. Res. Found., Washington, D.C.), herein incorporated by reference.

In making such changes, the hydropathic index of amino acids may be considered. The importance of the hydropathic amino acid index in conferring interactive biologic function on a protein is generally understood in the art (Kyte and Doolittle, (1982) J Mol Biol. 157(1):105-32). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It is known in the art that certain amino acids may be substituted by other amino acids having a similar hydropathic index or score and still result in a protein with similar biological activity, i.e., still obtain a biological functionally equivalent protein. Each amino acid has been assigned a hydropathic index on the basis of its hydrophobicity and charge characteristics (Kyte and Doolittle, ibid).

It is also understood in the art that the substitution of like amino acids can be made effectively on the basis of hydrophilicity. U.S. Pat. No. 4,554,101, states that the greatest local average hydrophilicity of a protein, as governed by the hydrophilicity of its adjacent amino acids, correlates with a biological property of the protein.

Alternatively, alterations may be made to the protein sequence of many proteins at the amino or carboxy terminus without substantially affecting activity. This can include insertions, deletions or alterations introduced by modern molecular methods, such as PCR, including PCR amplifications that alter or extend the protein coding sequence by virtue of inclusion of amino acid encoding sequences in the oligonucleotides utilized in the PCR amplification. Alternatively, the protein sequences added can include entire protein-coding sequences, such as those used commonly in the art to generate protein fusions. Such fusion proteins are often used to (1) increase expression of a protein of interest (2) introduce a binding domain, enzymatic activity or epitope to facilitate either protein purification, protein detection or other experimental uses known in the art (3) target secretion or translation of a protein to a subcellular organelle, such as the periplasmic space of Gram-negative bacteria, mitochondria or chloroplasts of plants or the endoplasmic reticulum of eukaryotic cells, the latter of which often results in glycosylation of the protein.

Variant nucleotide and amino acid sequences of the disclosure also encompass sequences derived from mutagenic and recombinogenic procedures such as DNA shuffling.

With such a procedure, one or more different polypeptides of the disclosure coding regions can be used to create a new polypeptide of possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, (1994) Proc. Natl. Acad. Sci. USA 91:10747-10751; Stemmer, (1994) Nature 370:389-391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291; and U.S. Pat. Nos. 5,605,793 and 5,837,458.

Antibodies to a MWLMV or JCSMV virus or modified MWLMV or JCSMV virus polypeptide of the embodiments or to variants or fragments thereof are also encompassed. The antibodies of the disclosure include polyclonal and monoclonal antibodies as well as fragments thereof which retain their ability to bind to a MWLMV or JCSMV virus or modified MWLMV or JCSMV virus polypeptide. An antibody, monoclonal antibody or fragment thereof is said to be capable of binding a molecule if it is capable of specifically reacting with the molecule to thereby bind the molecule to the antibody, monoclonal antibody or fragment thereof. The term “antibody” (Ab) or “monoclonal antibody” (Mab) is meant to include intact molecules as well as fragments or binding regions or domains thereof (such as, for example, Fab and F(ab).sub.2 fragments) which are capable of binding hapten. Such fragments are typically produced by proteolytic cleavage, such as papain or pepsin. Alternatively, hapten-binding fragments can be produced through the application of recombinant DNA technology or through synthetic chemistry. Methods for the preparation of the antibodies of the present disclosure are generally known in the art. For example, see, Antibodies, A Laboratory Manual, Ed Harlow and David Lane (eds.) Cold Spring Harbor Laboratory, N.Y. (1988), as well as the references cited therein. Standard reference works setting forth the general principles of immunology include: Klein, J. Immunology: The Science of Cell-Noncell Discrimination, John Wiley & Sons, N.Y. (1982); Dennett, et al., Monoclonal Antibodies, Hybridoma: A New Dimension in Biological Analyses, Plenum Press, N.Y. (1980) and Campbell, “Monoclonal Antibody Technology,” In Laboratory Techniques in Biochemistry and Molecular Biology, Vol. 13, Burdon, et al., (eds.), Elsevier, Amsterdam (1984). See also, U.S. Pat. Nos. 4,196,265; 4,609,893; 4,713,325; 4,714,681; 4,716,111; 4,716,117 and 4,720,459. Antibodies against MWLMV or JCSMV virus or modified MWLMV or JCSMV virus polypeptides or antigen-binding portions thereof can be produced by a variety of techniques, including conventional monoclonal antibody methodology, for example the standard somatic cell hybridization technique of Kohler and Milstein, (1975) Nature 256:495. Other techniques for producing monoclonal antibody can also be employed such as viral or oncogenic transformation of B lymphocytes. An animal system for preparing hybridomas is a murine system. Immunization protocols and techniques for isolation of immunized splenocytes for fusion are known in the art. Fusion partners (e.g., murine myeloma cells) and fusion procedures are also known. The antibody and monoclonal antibodies of the disclosure can be prepared by utilizing a MWLMV or JCSMV virus or modified MWLMV or JCSMV virus polypeptide as antigens.

A kit for detecting the presence of a MWLMV or JCSMV virus or modified MWLMV or JCSMV virus polypeptide or detecting the presence of a nucleotide sequence encoding a MWLMV or JCSMV virus or modified MWLMV or JCSMV virus polypeptide in a sample is provided. In one embodiment, the kit provides antibody-based reagents for detecting the presence of a MWLMV or JCSMV virus or modified MWLMV or JCSMV virus polypeptide in a tissue sample. In another embodiment, the kit provides labeled nucleic acid probes useful for detecting the presence of one or more polynucleotides encoding a MWLMV or JCSMV virus or modified MWLMV or JCSMV virus polypeptide. The kit is provided along with appropriate reagents and controls for carrying out a detection method, as well as instructions for use of the kit.

### VII. Compositions Comprising a Silencing Elements and a MWLMV or JCSMV Virus or Modified MWLMV or JCSMV Virus

A silencing element and a MWLMV, modified MWLMV, and MWLMV satellite VP, a JCSMV, a modified JCSMV, a MWLMV coat polypeptide, a MWLMV suppressor of RNA silencing, a satellite MWLMV coat polypeptide, a movement polypeptide, and/or a RNA-directed RNA polymerase polypeptide, as set forth in SEQ ID NOs: 117-122 and 140-144, may be provided as an external composition such as a spray or powder to the plant, plant part, seed, a plant insect pest, or an area of cultivation. In another example, a plant is transformed with a DNA construct or expression cassette for expression of a silencing element and a MWLMV or JCSMV virus or modified MWLMV or JCSMV virus. In another composition, a silencing element and a MWLMV or JCSMV virus or modified MWLMV or JCSMV virus, when ingested by an insect, can reduce the level of a target pest sequence and thereby control the pest (i.e., a Coleopteran plant pest including a Diabrotica plant pest, such as, D. virgifera virgifera, D. barberi, D. virgifera zeae, D. speciosa, or D. undecimpunctata howardi). It is recognized that the composition can comprise a cell (such as plant cell or a bacterial cell), in which the a polynucleotide encoding a silencing element and a polynucleotide encoding a MWLMV or JCSMV virus or modified MWLMV or JCSMV virus are stably incorporated into the genome and operably linked to promoters active in the cell. Compositions comprising a mixture of cells, some cells expressing at least one silencing element are also encompassed. In other embodiments, compositions comprising the silencing elements and the MWLMV or JCSMV virus or modified MWLMV or JCSMV virus are not contained in a cell. In such embodiments, the composition can be applied to an area inhabited by a plant insect pest. In one embodiment, the composition is applied externally to a plant (i.e., by spraying a field or area of cultivation) to protect the plant from the pest. Methods of applying nucleotides in such a manner are known to those of skill in the art.

The composition may further be formulated as bait. In this embodiment, the compositions comprise a food substance or an attractant which enhances the attractiveness of the composition to the pest.

The composition comprising the silencing element and a MWLMV or JCSMV virus or modified MWLMV or JCSMV virus may be formulated in an agriculturally suitable and/or environmentally acceptable carrier. Such carriers may be any material that the animal, plant or environment to be treated can tolerate. Furthermore, the carrier must be such that the composition remains effective at controlling a plant insect pest. Examples of such carriers include water, saline, Ringer's solution, dextrose or other sugar solutions, Hank's solution, and other aqueous physiologically balanced salt solutions, phosphate buffer, bicarbonate buffer and Tris buffer. In addition, the composition may include compounds that increase the half-life of a composition. Various insecticidal formulations can also be found in, for example, US Publications 2008/0275115, 2008/0242174, 2008/0027143, 2005/0042245, and 2004/0127520, each of which is herein incorporated by reference.

It is recognized that the polynucleotides comprising sequences encoding the silencing element and a MWLMV or JCSMV virus or modified MWLMV or JCSMV virus may be used to transform organisms to provide for host organism production of these components, and subsequent application of the host organism to the environment of the target pest(s). Such host organisms include baculoviruses, bacteria, and the like. In this manner, the combination of polynucleotides encoding the silencing element may be introduced via a suitable vector into a microbial host, and said host applied to the environment, or to plants or animals.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be stably incorporated into the genome of the cell (e.g., chromosome, plasmid, plastid, or mitochondrial DNA), converted into an autonomous replicon, or transiently expressed (e.g., transfected mRNA).

Microbial hosts that are known to occupy the “phytosphere” (phylloplane, phyllosphere, rhizosphere, and/or rhizoplana) of one or more crops of interest may be selected.

These microorganisms are selected so as to be capable of successfully competing in the particular environment with the wild-type microorganisms, provide for stable maintenance and expression of the sequences encoding the silencing element, and desirably, provide for improved protection of the components from environmental degradation and inactivation.

Such microorganisms include bacteria, algae, and fungi. Of particular interest are microorganisms such as bacteria, e.g., Pseudomonas, Erwinia, Serratia, Klebsiella, Xanthomonas, Streptomyces, Rhizobium, Rhodopseudomonas, Methylius, Agrobacterium, Acetobacter, Lactobacillus, Arthrobacter, Azotobacter, Leuconostoc, and Alcaligenes, fungi, particularly yeast, e.g., Saccharomyces, Cryptococcus, Kluyveromyces, Sporobolomyces, Rhodotorula, and Aureobasidium. Of particular interest are such phytosphere bacterial species as Pseudomonas syringae, Pseudomonas fluorescens, Serratia marcescens, Acetobacter xylinum, Agrobacteria, Rhodopseudomonas spheroides, Xanthomonas campestris, Rhizobium melioti, Alcaligenes entrophus, Clavibacter xyli and Azotobacter vinlandir, and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces rosues, S. odorus, Kluyveromyces veronae, and Aureobasidium pollulans. Of particular interest are the pigmented microorganisms.

A number of ways are available for introducing a polynucleotide encoding a silencing element and a polynucleotide encoding a MWLMV or JCSMV virus or modified MWLMV or JCSMV virus into the microbial host under conditions that allow for stable maintenance and expression of such nucleotide encoding sequences. For example, expression cassettes can be constructed which include the nucleotide constructs of interest operably linked with the transcriptional and translational regulatory signals for expression of the nucleotide constructs, and a nucleotide sequence homologous with a sequence in the host organism, whereby integration will occur, and/or a replication system that is functional in the host, whereby integration or stable maintenance will occur.

E. coli strain HT115 (DE3) is an RNaseII mutant bacterial host harboring a λDE3 lysogen, a source of T7 polymerase. Since E. coli is not naturally transformable, the ability to take up DNA or competency must be induced by chemical methods using divalent and multivalent cations, such as calcium, magnesium, manganese, rubidium, or hexamine cobalt (Maniatis, T., E. F. Fritsch, and J. Sambrook. Molecular Cloning, a Laboratory Manual, 1982) or an electrical shock method (Ausubel, et. al. Short Protocols in Molecular Biology, 5th Ed 2002). Timmons et. al (Gene. 2001) showed that ingestion of bacterially expressed dsRNAs can produce specific and potent genetic interference in Caenorhabditis elegans.

Transcriptional and translational regulatory signals include, but are not limited to, promoters, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (2000); Molecular Cloning: A Laboratory Manual (3^{rd} ed.; Cold Spring Harbor Laboratory Press, Plainview, N.Y.); Davis et al. (1980) Advanced Bacterial Genetics (Cold Spring Harbor Laboratory, Cold Spring Harbor, N.Y.); and the references cited therein.

Suitable host cells include the prokaryotes and the lower eukaryotes, such as fungi. Illustrative prokaryotes, both Gram-negative and Gram-positive, include Enterobacteriaceae, such as Escherichia, Erwinia, Shigella, Salmonella, and Proteus; Bacillaceae; Rhizobiceae, such as Rhizobium; Spirillaceae, such as photobacterium, Zymomonas, Serratia, Aeromonas, Vibrio, Desulfovibrio, Spirillum; Lactobacillaceae; Pseudomonadaceae, such as Pseudomonas and Acetobacter; Azotobacteraceae and Nitrobacteraceae. Among eukaryotes are fungi, such as Phycomycetes and Ascomycetes, which includes yeast, such as Saccharomyces and Schizosaccharomyces; and Basidiomycetes yeast, such as Rhodotorula, Aureobasidium, Sporobolomyces, and the like.

Characteristics of particular interest in selecting a host cell may include ease of introducing the coding sequence into the host, availability of expression systems, efficiency of expression, stability in the host, and the presence of auxiliary genetic capabilities. Characteristics of interest for use as a pesticide microcapsule include protective qualities, such as thick cell walls, pigmentation, and intracellular packaging or formation of inclusion bodies; leaf affinity; lack of mammalian toxicity; attractiveness to pests for ingestion; and the like. Other considerations include ease of formulation and handling, economics, storage stability, and the like.

Host organisms of particular interest include yeast, such as Rhodotorula spp., Aureobasidium spp., Saccharomyces spp., and Sporobolomyces spp., phylloplane organisms such as Pseudomonas spp., Erwinia spp., and Flavobacterium spp., and other such organisms, including Pseudomonas aeruginosa, Pseudomonas fluorescens, Saccharomyces cerevisiae, Bacillus thuringiensis, Escherichia coli, Bacillus subtilis, and the like.

The sequences encoding a silencing element and a MWLMV or JCSMV virus or modified MWLMV or JCSMV virus disclosed herein may be introduced into microorganisms that multiply on plants (epiphytes) to deliver these components to potential target pests. Epiphytes, for example, can be gram-positive or gram-negative bacteria.

The silencing element and a MWLMV or JCSMV virus or modified MWLMV or JCSMV virus may be fermented in a bacterial host and the resulting bacteria processed and used as a microbial spray in the same manner that Bacillus thuringiensis strains have been used as insecticidal sprays. Any suitable microorganism can be used for this purpose. By way of example, Pseudomonas has been used to express Bacillus thuringiensis endotoxins as encapsulated proteins and the resulting cells processed and sprayed as an insecticide Gaertner et al. (1993), in Advanced Engineered Pesticides, ed. L. Kim (Marcel Decker, Inc.).

Alternatively, the components are produced by introducing heterologous genes into a cellular host. Expression of the heterologous sequences results, directly or indirectly, in the intracellular production of the silencing element and a MWLMV or JCSMV virus or modified MWLMV or JCSMV virus. These compositions may then be formulated in accordance with conventional techniques for application to the environment hosting a target pest, e.g., soil, water, and foliage of plants. See, for example, EPA 0192319, and the references cited therein.

In one embodiment, a transformed microorganism can be formulated with an acceptable carrier into separate or combined compositions that are, for example, a suspension, a solution, an emulsion, a dusting powder, a dispersible granule, a wettable powder, and an emulsifiable concentrate, an aerosol, an impregnated granule, an adjuvant, a coatable paste, and also encapsulations in, for example, polymer substances.

Such compositions disclosed above may be obtained by the addition of a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors, or other preparations that influence plant growth. One or more agrochemicals including, but not limited to, herbicides, insecticides, fungicides, bactericides, nematicides, molluscicides, acaracides, plant growth regulators, harvest aids, and fertilizers, can be combined with carriers, surfactants or adjuvants customarily employed in the art of formulation or other components to facilitate product handling and application for particular target pests. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients of the composition (i.e., at least one silencing element) are normally applied in the form of compositions and can be applied to the crop area, plant, or seed to be treated. For example, the compositions may be applied to grain in preparation for or during storage in a grain bin or silo, etc. The compositions may be applied simultaneously or in succession with other compounds. Methods of applying an active ingredient or a composition that contains a silencing element and a MWLMV or JCSMV virus or modified MWLMV or JCSMV virus include, but are not limited to, foliar application, seed coating, and soil application. The number of applications and the rate of application depend on the intensity of infestation by the corresponding pest.

Suitable surface-active agents include, but are not limited to, anionic compounds such as a carboxylate of, for example, a metal; carboxylate of a long chain fatty acid; an N-acylsarcosinate; mono- or di-esters of phosphoric acid with fatty alcohol ethoxylates or salts of such esters; fatty alcohol sulfates such as sodium dodecyl sulfate, sodium octadecyl sulfate, or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate or dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g., polyoxyethylene sorbitan fatty acid esters, block copolymers of ethylene oxide and propylene oxide, acetylenic glycols such as 2,4,7,9-tetraethyl-5-decyn-4,7-diol, or ethoxylated acetylenic glycols. Examples of a cationic surface-active agent include, for instance, an aliphatic mono-, di-, or polyamine such as an acetate, naphthenate or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials include, but are not limited to, inorganic minerals such as kaolin, phyllosilicates, carbonates, sulfates, phosphates, or botanical materials such as cork, powdered corncobs, peanut hulls, rice hulls, and walnut shells.

The compositions comprising a silencing element and a MWLMV or JCSMV virus or modified MWLMV or JCSMV virus may be in a suitable form for direct application or as a concentrate of primary composition that requires dilution with a suitable quantity of water or other dilutant before application.

The compositions (including the transformed microorganisms) may be applied to the environment of an insect pest (such as a Coleoptera plant pest or a Diabrotica plant pest) by, for example, spraying, atomizing, dusting, scattering, coating or pouring, introducing into or on the soil, introducing into irrigation water, by seed treatment or general application or dusting at the time when the pest has begun to appear or before the appearance of pests as a protective measure. For example, the composition(s) and/or transformed microorganism(s) may be mixed with grain to protect the grain during storage. It is generally important to obtain good control of pests in the early stages of plant growth, as this is the time when the plant can be most severely damaged. The compositions can conveniently contain another insecticide if this is thought necessary. In an embodiment, the composition(s) is applied directly to the soil, at a time of planting, in granular form of a composition of a carrier and dead cells of a Bacillus strain or transformed microorganism of the invention. Another embodiment is a granular form of a composition comprising an agrochemical such as, for example, an herbicide, an insecticide, a fertilizer, in an inert carrier, and dead cells of a Bacillus strain or transformed microorganism of the invention.

IX. Plants, Plant Parts, and Methods of Introducing Sequences into Plants

In one embodiment, the methods involve introducing a polynucleotide into a plant. “Introducing” is intended to mean presenting to the plant the polynucleotide in such a manner that the sequence gains access to the interior of a cell of the plant. The methods disclosed herein do not depend on a particular method for introducing a sequence into a plant, only that the polynucleotide or polypeptides gains access to the interior of at least one cell of the plant. Methods for introducing polynucleotides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lecl transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro CellDev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990)Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, a silencing element and a MWLMV or JCSMV virus or modified MWLMV or JCSMV virus polynucleotides may be provided to a plant using a variety of transient transformation methods. Such transient transformation methods include, but are not limited to, the introduction of the protein or variants or fragments thereof directly into the plant or the introduction of the transcript into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, polynucleotides can be transiently transformed into the plant using techniques known in the art. Such techniques include viral vector systems and the precipitation of the polynucleotide in a manner that precludes subsequent release of the DNA. Thus, the transcription from the particle-bound DNA can occur, but the frequency with which it is released to become integrated into the genome is greatly reduced. Such methods include the use of particles coated with polyethylimine (PEI; Sigma # P3143).

In other embodiments, the polynucleotides disclosed herein may be introduced into plants by contacting plants with a virus or viral nucleic acids. Generally, such methods involve incorporating a nucleotide construct of the invention within a viral DNA or RNA molecule. Further, it is recognized that promoters also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of interest can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of interest, for example, an expression cassette of disclosed herein, stably incorporated into their genome.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the embodiments, provided that these parts comprise the introduced polynucleotides.

The present embodiments may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbiapulcherrima), and chrysanthemum.

Conifers that may be employed in practicing the present embodiments include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants and sugarcane plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

### X. Stacking of Traits in Transgenic Plant

Transgenic plants may comprise a stack of a polynucleotide encoding a silencing element and a polynucleotide encoding a MWLMV or JCSMV virus or modified MWLMV or JCSMV virus, such as the sequences as set forth in SEQ ID NOS.: 1-22, or variants or fragments thereof, or complements thereof, as disclosed herein with one or more additional polynucleotides resulting in the production or suppression of multiple polypeptide sequences. In one embodiment, the transgenic plant may comprise the stack with a polynucleotide encoding a MWLMV or JCSMV virus or modified MWLMV or JCSMV virus. Transgenic plants comprising stacks of polynucleotide sequences may be obtained by either or both of traditional breeding methods or through genetic engineering methods. These methods include, but are not limited to, breeding individual lines each comprising a polynucleotide of interest, transforming a transgenic plant comprising an expression construct comprising a polynucleotide encoding a MWLMV or JCSMV virus or modified MWLMV or JCSMV virus and various silencing elements with a subsequent gene and co-transformation of genes into a single plant cell. As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. Co-transformation of polynucleotides can be carried out using single transformation vectors comprising multiple polynucleotides or polynucleotides carried separately on multiple vectors. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. It is further recognized that polynucleotide sequences can be stacked at a desired genomic location using a site-specific recombination system. See, for example, WO 1999/25821, WO 1999/25854, WO 1999/25840, WO 1999/25855 and WO 1999/25853.

Transgenes useful for stacking include but are not limited to: transgenes that confer resistance to a herbicide; transgenes that confer or contribute to an altered grain characteristic; genes that control male-sterility; genes that create a site for site specific dna integration; genes that affect abiotic stress resistance; genes that confer increased yield genes that confer plant digestibility; and transgenes that confer resistance to insects or disease.

In some embodiments the various target polynucleotides, alone or stacked with one or more additional insect resistance traits can be stacked with one or more additional input traits (e.g., herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, and the like). Thus, the polynucleotide embodiments can be used to provide a complete agronomic package of improved crop quality with the ability to flexibly and cost effectively control any number of agronomic pests.

Examples of transgenes that confer resistance to insects include genes encoding a Bacillus thuringiensis protein, a derivative thereof or a synthetic polypeptide modeled thereon. See, for example, Geiser, et al., (1986) Gene 48:109, who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC® Accession Numbers 40098, 67136, 31995 and 31998. Other non-limiting examples of Bacillus thuringiensis transgenes being genetically engineered are given in the following patents and patent applications and hereby are incorporated by reference for this purpose: U.S. Pat. Nos. 5,188,960; 5,689,052; 5,880,275; 5,986,177; 6,023,013, 6,060,594, 6,063,597, 6,077,824, 6,620,988, 6,642,030, 6,713,259, 6,893,826, 7,105,332; 7,179,965, 7,208,474; 7,227,056, 7,288,643, 7,323,556, 7,329,736, 7,449,552, 7,468,278, 7,510,878, 7,521,235, 7,544,862, 7,605,304, 7,696,412, 7,629,504, 7,705,216, 7,772,465, 7,790,846, 7,858,849 and WO 1991/14778; WO 1999/31248; WO 2001/12731; WO 1999/24581 and WO 1997/40162.

Genes encoding pesticidal proteins may also be stacked including but are not limited to: insecticidal proteins from Pseudomonas sp. such as PSEEN3174 (Monalysin, (2011) PLoS Pathogens, 7:1-13), from Pseudomonas protegens strain CHAO and Pf-5 (previously fluorescens) (Pechy-Tarr, (2008) Environmental Microbiology 10:2368-2386: GenBank Accession No. EU400157); from Pseudomonas Taiwanensis (Liu, et al., (2010) J Agric. Food Chem. 58:12343-12349) and from Pseudomonas pseudoalcligenes (Zhang, et al., (2009) Annals of Microbiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxinology Journal 3:101-118 and Morgan, et al., (2001) Applied and Envir. Micro. 67:2062-2069), U.S. Pat. Nos. 6,048,838, and 6,379,946; a PIP-1 polypeptide of US Patent Publication US20140007292; an AfIP-1A and/or AflP-1B polypeptide of US Patent Publication US20140033361; a PHI-4 polypeptide of US Patent Publication US20140274885 and US20160040184; a PIP-47 polypeptide of PCT Publication Number WO2015/023846, a PIP-72 polypeptide of PCT Publication Number WO2015/038734; a PtlP-50 polypeptide and a PtlP-65 polypeptide of PCT Publication Number WO2015/120270; a PtIP-83 polypeptide of PCT Publication Number WO2015/120276; a PtIP-96 polypeptide of PCT Serial Number PCT/US 15/55502; an IPD079 polypeptide of U.S. Ser. No. 62/201,977; an IPD082 polypeptide of U.S. Ser. No. 62/269,482; and 6-endotoxins including, but not limited to, the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry 51 and Cry55 classes of 6-endotoxin genes and the B. thuringiensis cytolytic Cyt1 and Cyt2 genes. Members of these classes of B. thuringiensis insecticidal proteins include, but are not limited to Cry1Aa1 (Accession # AAA22353); Cry1Aa2 (Accession # Accession # AAA22552); Cry1Aa3 (Accession # BAA00257); Cry1Aa4 (Accession # CAA31886); Cry1Aa5 (Accession # BAA04468); Cry1Aa6 (Accession # AAA86265); Cry1Aa7 (Accession # AAD46139); Cry1Aa8 (Accession #126149); Cry1Aa9 (Accession # BAA77213); Cry1Aa10 (Accession # AAD55382); Cry1Aa1l (Accession # CAA70856); Cry1Aa12 (Accession # AAP80146); Cry1Aa13 (Accession # AAM44305); Cry1Aa14 (Accession # AAP40639); Cry1Aa15 (Accession # AAY66993); Cry1Aa16 (Accession # HQ439776); Cry1Aa17 (Accession # HQ439788); Cry1Aa18 (Accession # HQ439790); Cry1Aa19 (Accession # HQ685121); Cry1Aa20 (Accession # JF340156); Cry1Aa21 (Accession # JN651496); Cry1Aa22 (Accession # KC158223); Cry1Ab1 (Accession # AAA22330); Cry1Ab2 (Accession # AAA22613); Cry1Ab3 (Accession # AAA22561); Cry1Ab4 (Accession # BAA00071); Cry1Ab5 (Accession # CAA28405); Cry1Ab6 (Accession # AAA22420); Cry1Ab7 (Accession # CAA31620); Cry1Ab8 (Accession # AAA22551); Cry1Ab9 (Accession # CAA38701); Cry1Ab10 (Accession # A29125); Cry1Ab11 (Accession # I12419); Cry1Ab12 (Accession # AAC64003); Cry1Ab13 (Accession # AAN76494); Cry1Ab14 (Accession # AAG16877); Cry1Ab15 (Accession # AA013302); Cry1Ab16 (Accession # AAK55546); Cry1Ab17 (Accession # AAT46415); Cry1Ab18 (Accession # AAQ88259); Cry1Ab19 (Accession # AAW31761); Cry1Ab20 (Accession # ABB72460); Cry1Ab21 (Accession # ABS18384); Cry1Ab22 (Accession # ABW87320); Cry1Ab23 (Accession # HQ439777); Cry1Ab24 (Accession # HQ439778); Cry1Ab25 (Accession # HQ685122); Cry1Ab26 (Accession # HQ847729); Cry1Ab27 (Accession # JN135249); Cry1Ab28 (Accession # JN135250); Cry1Ab29 (Accession # JN135251); Cry1Ab30 (Accession # JN135252); Cry1Ab31 (Accession # JN135253); Cry1Ab32 (Accession # JN135254); Cry1Ab33 (Accession # AAS93798); Cry1Ab34 (Accession # KC156668); Cry1Ab-like (Accession # AAK14336); Cry1Ab-like (Accession # AAK14337); Cry1Ab-like (Accession # AAK14338); Cry1Ab-like (Accession # ABG88858); Cry1Ac1 (Accession # AAA22331); Cry1Ac2 (Accession # AAA22338); Cry1Ac3 (Accession # CAA38098); Cry1Ac4 (Accession # AAA73077); Cry1Ac5 (Accession # AAA22339); Cry1Ac6 (Accession # AAA86266); Cry1Ac7 (Accession # AAB46989); Cry1Ac8 (Accession # AAC44841); Cry1Ac9 (Accession # AAB49768); Cry1Ac10 (Accession # CAA05505); Cry1Ac11 (Accession # CAA10270); Cry1Ac12 (Accession #112418); Cry1Ac13 (Accession # AAD38701); Cry1Ac14 (Accession # AAQ06607); Cry1Ac15 (Accession # AAN07788); Cry1Ac16 (Accession # AAU87037); Cry1Ac17 (Accession # AAX18704); Cry1Ac18 (Accession # AAY88347); Cry1Ac19 (Accession # ABD37053); Cry1Ac20 (Accession # ABB89046); Cry1Ac21 (Accession # AAY66992); Cry1Ac22 (Accession # ABZ01836); Cry1Ac23 (Accession # CAQ30431); Cry1Ac24 (Accession # ABL01535); Cry1Ac25 (Accession # FJ513324); Cry1Ac26 (Accession # FJ617446); Cry1Ac27 (Accession # FJ617447); Cry1Ac28 (Accession # ACM90319); Cry1Ac29 (Accession # DQ438941); Cry1Ac30 (Accession # GQ227507); Cry1Ac31 (Accession # GU446674); Cry1Ac32 (Accession # HM061081); Cry1Ac33 (Accession # GQ866913); Cry1Ac34 (Accession # HQ230364); Cry1Ac35 (Accession # JF340157); Cry1Ac36 (Accession # JN387137); Cry1Ac37 (Accession # JQ317685); Cry1Ad1 (Accession # AAA22340); Cry1Ad2 (Accession # CAA01880); Cry1Ae1 (Accession # AAA22410); Cry1Af1 (Accession # AAB82749); Cry1Ag1 (Accession # AAD46137); Cry1Ah1 (Accession # AAQ14326); Cry1Ah2 (Accession # ABB76664); Cry1Ah3 (Accession # HQ439779); Cry1Ai1 (Accession # AA039719); Cry1Ai2 (Accession # HQ439780); Cry1A-like (Accession # AAK14339); Cry1Ba1 (Accession # CAA29898); Cry1Ba2 (Accession # CAA65003); Cry1Ba3 (Accession # AAK63251); Cry1Ba4 (Accession # AAK51084); Cry1Ba5 (Accession # AB020894); Cry1Ba6 (Accession # ABL60921); Cry1Ba7 (Accession # HQ439781); Cry1Bb1 (Accession # AAA22344); Cry1Bb2 (Accession # HQ439782); Cry1Bc1 (Accession # CAA86568); Cry1Bd1 (Accession # AAD10292); Cry1Bd2 (Accession # AAM93496); Cry1Be1 (Accession # AAC32850); Cry1Be2 (Accession # AAQ52387); Cry1Be3 (Accession # ACV96720); Cry1Be4 (Accession # HM070026); Cry1Bf1 (Accession # CAC50778); Cry1Bf2 (Accession # AAQ52380); Cry1Bg1 (Accession # AA039720); Cry1Bh1 (Accession # HQ589331); Cry1Bi1 (Accession # KC156700); Cry1Ca1 (Accession # CAA30396); Cry1Ca2 (Accession # CAA31951); Cry1Ca3 (Accession # AAA22343); Cry1Ca4 (Accession # CAA01886); Cry1Ca5 (Accession # CAA65457); Cry1Ca6 [1] (Accession # AAF37224); Cry1Ca7 (Accession # AAG50438); Cry1Ca8 (Accession # AAM00264); Cry1Ca9 (Accession # AAL79362); Cry1Ca10 (Accession # AAN16462); Cry1Ca11 (Accession # AAX53094); Cry1Ca12 (Accession # HM070027); Cry1Ca13 (Accession # HQ412621); Cry1Ca14 (Accession # JN651493); Cry1Cb1 (Accession # M97880); Cry1Cb2 (Accession # AAG35409); Cry1Cb3 (Accession # ACD50894); Cry1Cb-like (Accession # AAX63901); Cry1Da1 (Accession # CAA38099); Cry1Da2 (Accession #176415); Cry1Da3 (Accession # HQ439784); Cry1Db1 (Accession # CAA80234); Cry1Db2 (Accession # AAK48937); Cry1Dc1 (Accession # ABK35074); Cry1Ea1 (Accession # CAA37933); Cry1Ea2 (Accession # CAA39609); Cry1Ea3 (Accession # AAA22345); Cry1Ea4 (Accession # AAD04732); Cry1Ea5 (Accession # A15535); Cry1Ea6 (Accession # AAL50330); Cry1Ea7 (Accession # AAW72936); Cry1Ea8 (Accession # ABX11258); Cry1Ea9 (Accession # HQ439785); Cry1Ea10 (Accession # ADR00398); Cry1Ea11 (Accession # JQ652456); Cry1Eb1 (Accession # AAA22346); Cry1Fa1 (Accession # AAA22348); Cry1Fa2 (Accession # AAA22347); Cry1Fa3 (Accession # HM070028); Cry1Fa4 (Accession # HM439638); Cry1Fb1 (Accession # CAA80235); Cry1Fb2 (Accession # BAA25298); Cry1Fb3 (Accession # AAF21767); Cry1Fb4 (Accession # AAC10641); Cry1Fb5 (Accession # AAO13295); Cry1Fb6 (Accession # ACD50892); Cry1Fb7 (Accession # ACD50893); Cry1Ga1 (Accession # CAA80233); Cry1Ga2 (Accession # CAA70506); Cry1Gb1 (Accession # AAD10291); Cry1Gb2 (Accession # AA013756); Cry1Gc1 (Accession # AAQ52381); Cry1Ha1 (Accession # CAA80236); Cry1Hb1 (Accession # AAA79694); Cry1Hb2 (Accession # HQ439786); Cry1H-like (Accession # AAF01213); Cry1Ia1 (Accession # CAA44633); Cry1Ia2 (Accession # AAA22354); Cry1Ia3 (Accession # AAC36999); Cry1Ia4 (Accession # AAB00958); Cry1Ia5 (Accession # CAA70124); Cry1Ia6 (Accession # AAC26910); Cry1Ia7 (Accession # AAM73516); Cry1Ia8 (Accession # AAK66742); Cry1Ia9 (Accession # AAQ08616); Cry1Ia10 (Accession # AAP86782); Cry1Ia11 (Accession # CAC85964); Cry1Ia12 (Accession # AAV53390); Cry1Ia13 (Accession # ABF83202); Cry1Ia14 (Accession # ACG63871); Cry1Ia15 (Accession # FJ617445); Cry1Ia16 (Accession # FJ617448); Cry1Ia17 (Accession # GU989199); Cry1Ia18 (Accession # ADK23801); Cry1Ia19 (Accession # HQ439787); Cry1Ia20 (Accession #JQ228426); Cry1Ia21 (Accession # JQ228424); Cry1Ia22 (Accession # JQ228427); Cry1Ia23 (Accession # JQ228428); Cry1Ia24 (Accession # JQ228429); Cry1Ia25 (Accession # JQ228430); Cry1Ia26 (Accession # JQ228431); Cry1Ia27 (Accession # JQ228432); Cry1Ia28 (Accession # JQ228433); Cry1Ia29 (Accession # JQ228434); Cry1Ia30 (Accession # JQ317686); Cry1Ia31 (Accession # JX944038); Cry1Ia32 (Accession # JX944039); Cry1Ia33 (Accession # JX944040); Cry1Ib1 (Accession # AAA82114); Cry1Ib2 (Accession # ABW88019); Cry1Ib3 (Accession # ACD75515); Cry1Ib4 (Accession # HM051227); Cry1Ib5 (Accession # HM070028); Cry1Ib6 (Accession # ADK38579); Cry1Ib7 (Accession # JN571740); Cry1Ib8 (Accession # JN675714); Cry1Ib9 (Accession # JN675715); Cry1Ib10 (Accession # JN675716); Cry1Ib11 (Accession # JQ228423); Cry1Ic1 (Accession # AAC62933); Cry1Ic2 (Accession # AAE71691); Cry1Id1 (Accession # AAD44366); Cry1Id2 (Accession # JQ228422); Cry1Ie1 (Accession # AAG43526); Cry1Ie2 (Accession # HM439636); Cry1Ie3 (Accession # KC156647); Cry1Ie4 (Accession # KC156681); Cry1If1 (Accession # AAQ52382); Cry1Ig1 (Accession # KC156701); Cry1I-like (Accession # AAC31094); Cry1I-like (Accession # ABG88859); Cry1Ja1 (Accession # AAA22341); Cry1Ja2 (Accession # HM070030); Cry1Ja3 (Accession # JQ228425); Cry1Jb1 (Accession # AAA98959); Cry1Jc1 (Accession # AAC31092); Cry1Jc2 (Accession # AAQ52372); Cry1Jd1 (Accession # CAC50779); Cry1Ka1 (Accession # AAB00376); Cry1Ka2 (Accession # HQ439783); Cry1La1 (Accession # AAS60191); Cry1La2 (Accession # HM070031); Cry1Ma1 (Accession # FJ884067); Cry1Ma2 (Accession # KC156659); Cry1Na1 (Accession # KC156648); Cry1Nb1 (Accession # KC156678); Cry1-like (Accession # AAC31091); Cry2Aa1 (Accession # AAA22335); Cry2Aa2 (Accession # AAA83516); Cry2Aa3 (Accession # D86064); Cry2Aa4 (Accession # AAC04867); Cry2Aa5 (Accession # CAA10671); Cry2Aa6 (Accession # CAA10672); Cry2Aa7 (Accession # CAA10670); Cry2Aa8 (Accession # AA013734); Cry2Aa9 (Accession # AA013750); Cry2Aa10 (Accession # AAQ04263); Cry2Aa11 (Accession # AAQ52384); Cry2Aa12 (Accession # ABI83671); Cry2Aa13 (Accession # ABL01536); Cry2Aa14 (Accession # ACF04939); Cry2Aa15 (Accession # JN426947); Cry2Ab1 (Accession # AAA22342); Cry2Ab2 (Accession # CAA39075); Cry2Ab3 (Accession # AAG36762); Cry2Ab4 (Accession # AA013296); Cry2Ab5 (Accession # AAQ04609); Cry2Ab6 (Accession # AAP59457); Cry2Ab7 (Accession # AAZ66347); Cry2Ab8 (Accession # ABC95996); Cry2Ab9 (Accession # ABC74968); Cry2Ab10 (Accession # EF157306); Cry2Ab11 (Accession # CAM84575); Cry2Ab12 (Accession # ABM21764); Cry2Ab13 (Accession # ACG76120); Cry2Ab14 (Accession #ACG76121); Cry2Ab15 (Accession # HM037126); Cry2Ab16 (Accession # GQ866914); Cry2Ab17 (Accession # HQ439789); Cry2Ab18 (Accession # JN135255); Cry2Ab19 (Accession # JN135256); Cry2Ab20 (Accession # JN135257); Cry2Ab21 (Accession # JN135258); Cry2Ab22 (Accession # JN135259); Cry2Ab23 (Accession # JN135260); Cry2Ab24 (Accession # JN135261); Cry2Ab25 (Accession # JN415485); Cry2Ab26 (Accession # JN426946); Cry2Ab27 (Accession # JN415764); Cry2Ab28 (Accession # JN651494); Cry2Ac1 (Accession # CAA40536); Cry2Ac2 (Accession # AAG35410); Cry2Ac3 (Accession # AAQ52385); Cry2Ac4 (Accession # ABC95997); Cry2Ac5 (Accession # ABC74969); Cry2Ac6 (Accession # ABC74793); Cry2Ac7 (Accession # CAL18690); Cry2Ac8 (Accession # CAM09325); Cry2Ac9 (Accession # CAM09326); Cry2Ac10 (Accession # ABN15104); Cry2Ac1l (Accession # CAM83895); Cry2Ac12 (Accession # CAM83896); Cry2Ad1 (Accession # AAF09583); Cry2Ad2 (Accession # ABC86927); Cry2Ad3 (Accession # CAK29504); Cry2Ad4 (Accession # CAM32331); Cry2Ad5 (Accession # CAO78739); Cry2Ae1 (Accession # AAQ52362); Cry2Af1 (Accession # AB030519); Cry2Af2 (Accession # GQ866915); Cry2Ag1 (Accession # ACH91610); Cry2Ah1 (Accession # EU939453); Cry2Ah2 (Accession # ACL80665); Cry2Ah3 (Accession # GU073380); Cry2Ah4 (Accession # KC156702); Cry2Ai1 (Accession # FJ788388); Cry2Aj (Accession #); Cry2Ak1 (Accession # KC156660); Cry2Ba1 (Accession # KC156658); Cry3Aa1 (Accession # AAA22336); Cry3Aa2 (Accession # AAA22541); Cry3Aa3 (Accession # CAA68482); Cry3Aa4 (Accession # AAA22542); Cry3Aa5 (Accession # AAA50255); Cry3Aa6 (Accession # AAC43266); Cry3Aa7 (Accession # CAB41411); Cry3Aa8 (Accession # AAS79487); Cry3Aa9 (Accession # AAW05659); Cry3Aa10 (Accession # AAU29411); Cry3Aa11 (Accession # AAW82872); Cry3Aa12 (Accession # ABY49136); Cry3Ba1 (Accession # CAA34983); Cry3Ba2 (Accession # CAA00645); Cry3Ba3 (Accession # JQ397327); Cry3Bb1 (Accession # AAA22334); Cry3Bb2 (Accession # AAA74198); Cry3Bb3 (Accession #115475); Cry3Ca1 (Accession # CAA42469); Cry4Aa1 (Accession # CAA68485); Cry4Aa2 (Accession # BAA00179); Cry4Aa3 (Accession # CAD30148); Cry4Aa4 (Accession # AFB18317); Cry4A-like (Accession # AAY96321); Cry4Ba1 (Accession # CAA30312); Cry4Ba2 (Accession # CAA30114); Cry4Ba3 (Accession # AAA22337); Cry4Ba4 (Accession # BAA00178); Cry4Ba5 (Accession # CAD30095); Cry4Ba-like (Accession # ABC47686); Cry4Ca1 (Accession # EU646202); Cry4Cb1 (Accession # FJ403208); Cry4Cb2 (Accession # FJ597622); Cry4Cc1 (Accession # FJ403207); Cry5Aa1 (Accession # AAA67694); Cry5Ab1 (Accession # AAA67693); Cry5Ac1 (Accession #134543); Cry5Ad1 (Accession # ABQ82087); Cry5Ba1 (Accession # AAA68598); Cry5Ba2 (Accession # ABW88931); Cry5Ba3 (Accession # AFJ04417); Cry5Ca1 (Accession # HM461869); Cry5Ca2 (Accession # ZP_04123426); Cry5Da1 (Accession # HM461870); Cry5Da2 (Accession # ZP_04123980); Cry5Ea1 (Accession #5 HM485580); Cry5Ea2 (Accession # ZP_04124038); Cry6Aa1 (Accession # AAA22357); Cry6Aa2 (Accession # AAM46849); Cry6Aa3 (Accession # ABH03377); Cry6Ba1 (Accession # AAA22358); Cry7Aa1 (Accession # AAA22351); Cry7Ab1 (Accession # AAA21120); Cry7Ab2 (Accession # AAA21121); Cry7Ab3 (Accession # ABX24522); Cry7Ab4 (Accession # EU380678); Cry7Ab5 (Accession # ABX79555); Cry7Ab6 (Accession # ACI44005); Cry7Ab7 (Accession # ADB89216); Cry7Ab8 (Accession # GU145299); Cry7Ab9 (Accession # ADD92572); Cry7Ba1 (Accession # ABB70817); Cry7Bb1 (Accession # KC156653); Cry7Ca1 (Accession # ABR67863); Cry7Cb1 (Accession # KC156698); Cry7Da1 (Accession # ACQ99547); Cry7Da2 (Accession # HM572236); Cry7Da3 (Accession # KC156679); Cry7Ea1 (Accession # HM035086); Cry7Ea2 (Accession # HM132124); Cry7Ea3 (Accession # EEM19403); Cry7Fa1 (Accession # HM035088); Cry7Fa2 (Accession # EEM19090); Cry7Fb1 (Accession # HM572235); Cry7Fb2 (Accession # KC156682); Cry7Ga1 (Accession # HM572237); Cry7Ga2 (Accession # KC156669); Cry7Gb1 (Accession # KC156650); Cry7Gc1 (Accession # KC156654); Cry7Gd1 (Accession # KC156697); Cry7Ha1 (Accession # KC156651); Cry7Ia1 (Accession # KC156665); Cry7Ja1 (Accession # KC156671); Cry7Ka1 (Accession # KC156680); Cry7Kb1 (Accession # BAM99306); Cry7La1 (Accession # BAM99307); Cry8Aa1 (Accession # AAA21117); Cry8Ab1 (Accession # EU044830); Cry8Ac1 (Accession # KC156662); Cry8Ad1 (Accession # KC156684); Cry8Ba1 (Accession # AAA21118); Cry8Bb1 (Accession # CAD57542); Cry8Bc1 (Accession # CAD57543); Cry8Ca1 (Accession # AAA21119); Cry8Ca2 (Accession # AAR98783); Cry8Ca3 (Accession # EU625349); Cry8Ca4 (Accession # ADB54826); Cry8Da1 (Accession # BAC07226); Cry8Da2 (Accession # BD133574); Cry8Da3 (Accession # BD133575); Cry8Db1 (Accession # BAF93483); Cry8Ea1 (Accession # AAQ73470); Cry8Ea2 (Accession # EU047597); Cry8Ea3 (Accession # KC855216); Cry8Fa1 (Accession # AAT48690); Cry8Fa2 (Accession # HQ174208); Cry8Fa3 (Accession # AFH78109); Cry8Ga1 (Accession # AAT46073); Cry8Ga2 (Accession # ABC42043); Cry8Ga3 (Accession # FJ198072); Cry8Ha1 (Accession # AAW81032); Cry8Ia1 (Accession # EU381044); Cry8Ia2 (Accession # GU073381); Cry8Ia3 (Accession # HM044664); Cry8Ia4 (Accession # KC156674); Cry8Ib1 (Accession # GU325772); Cry8Ib2 (Accession # KC156677); Cry8Ja1 (Accession # EU625348); Cry8Ka1 (Accession # FJ422558); Cry8Ka2 (Accession # ACN87262); Cry8Kb1 (Accession # HM123758); Cry8Kb2 (Accession # KC156675); Cry8La1 (Accession # GU325771); Cry8Ma1 (Accession # HM044665); Cry8Ma2 (Accession # EEM86551); Cry8Ma3 (Accession # HM210574); Cry8Na1 (Accession # HM640939); Cry8Pa1 (Accession # HQ388415); Cry8Qa1 (Accession # HQ441166); Cry8Qa2 (Accession # KC152468); Cry8Ra1 (Accession # AFP87548); Cry8Sa1 (Accession # JQ740599); Cry8Ta1 (Accession # KC156673); Cry8-like (Accession # FJ770571); Cry8-like (Accession # ABS53003); Cry9Aa1 (Accession # CAA41122); Cry9Aa2 (Accession # CAA41425); Cry9Aa3 (Accession # GQ249293); Cry9Aa4 (Accession # GQ249294); Cry9Aa5 (Accession # JX174110); Cry9Aa like (Accession # AAQ52376); Cry9Ba1 (Accession # CAA52927); Cry9Ba2 (Accession # GU299522); Cry9Bb1 (Accession # AAV28716); Cry9Ca1 (Accession # CAA85764); Cry9Ca2 (Accession # AAQ52375); Cry9Da1 (Accession # BAA19948); Cry9Da2 (Accession # AAB97923); Cry9Da3 (Accession # GQ249293); Cry9Da4 (Accession # GQ249297); Cry9Db1 (Accession # AAX78439); Cry9Dc1 (Accession # KC156683); Cry9Ea1 (Accession # BAA34908); Cry9Ea2 (Accession # AAO12908); Cry9Ea3 (Accession # ABM21765); Cry9Ea4 (Accession # ACE88267); Cry9Ea5 (Accession # ACF04743); Cry9Ea6 (Accession # ACG63872); Cry9Ea7 (Accession # FJ380927); Cry9Ea8 (Accession # GQ249292); Cry9Ea9 (Accession # JN651495); Cry9Eb1 (Accession # CAC50780); Cry9Eb2 (Accession # GQ249298); Cry9Eb3 (Accession # KC156646); Cry9Ec1 (Accession # AAC63366); Cry9Ed1 (Accession # AAX78440); Cry9Ee1 (Accession # GQ249296); Cry9Ee2 (Accession # KC156664); Cry9Fa1 (Accession # KC156692); Cry9Ga1 (Accession # KC156699); Cry9-like (Accession # AAC63366); Cry10Aa1 (Accession # AAA22614); Cry10Aa2 (Accession # E00614); Cry10Aa3 (Accession # CAD30098); Cry10Aa4 (Accession # AFB18318); Cry10A-like (Accession # DQ167578); Cry11Aa1 (Accession # AAA22352); Cry11Aa2 (Accession # AAA22611); Cry11Aa3 (Accession # CAD30081); Cry11Aa4 (Accession # AFB18319); Cry11Aa-like (Accession # DQ166531); Cry11Ba1 (Accession # CAA60504); Cry11Bb1 (Accession # AAC97162); Cry11Bb2 (Accession # HM068615); Cry12Aa1 (Accession # AAA22355); Cry13Aa1 (Accession # AAA22356); Cry14Aa1 (Accession # AAA21516); Cry14Ab1 (Accession # KC156652); Cry15Aa1 (Accession # AAA22333); Cry16Aa1 (Accession # CAA63860); Cry17Aa1 (Accession # CAA67841); Cry18Aa1 (Accession # CAA67506); Cry18Ba1 (Accession # AAF89667); Cry18Ca1 (Accession # AAF89668); Cry19Aa1 (Accession # CAA68875); Cry19Ba1 (Accession # BAA32397); Cry19Ca1 (Accession # AFM37572); Cry20Aa1 (Accession # AAB93476); Cry20Ba1 (Accession # ACS93601); Cry20Ba2 (Accession # KC156694); Cry20-like (Accession # GQ144333); Cry21Aa1 (Accession #132932); Cry21Aa2 (Accession #166477); Cry21Ba1 (Accession # BAC06484); Cry21Ca1 (Accession # JF521577); Cry21Ca2 (Accession # KC156687); Cry21Da1 (Accession # JF521578); Cry22Aa1 (Accession #134547); Cry22Aa2 (Accession # CAD43579); Cry22Aa3 (Accession # ACD93211); Cry22Ab1 (Accession # AAK50456); Cry22Ab2 (Accession # CAD43577); Cry22Ba1 (Accession # CAD43578); Cry22Bb1 (Accession # KC156672); Cry23Aa1 (Accession # AAF76375); Cry24Aa1 (Accession # AAC61891); Cry24Ba1 (Accession # BAD32657); Cry24Ca1 (Accession # CAJ43600); Cry25Aa1 (Accession # AAC61892); Cry26Aa1 (Accession # AAD25075); Cry27Aa1 (Accession # BAA82796); Cry28Aa1 (Accession # AAD24189); Cry28Aa2 (Accession # AAG00235); Cry29Aa1 (Accession # CAC80985); Cry30Aa1 (Accession # CAC80986); Cry30Ba1 (Accession # BAD00052); Cry30Ca1 (Accession # BAD67157); Cry30Ca2 (Accession # ACU24781); Cry30Da1 (Accession # EF095955); Cry30Db1 (Accession # BAE80088); Cry30Ea1 (Accession # ACC95445); Cry30Ea2 (Accession # FJ499389); Cry30Fa1 (Accession # ACI22625); Cry30Ga1 (Accession # ACG60020); Cry30Ga2 (Accession # HQ638217); Cry31Aa1 (Accession # BAB11757); Cry31Aa2 (Accession # AAL87458); Cry31Aa3 (Accession # BAE79808); Cry31Aa4 (Accession # BAF32571); Cry31Aa5 (Accession # BAF32572); Cry31Aa6 (Accession # BAI44026); Cry31Ab1 (Accession # BAE79809); Cry31Ab2 (Accession # BAF32570); Cry31Ac1 (Accession # BAF34368); Cry31Ac2 (Accession # AB731600); Cry31Ad1 (Accession # BAI44022); Cry32Aa1 (Accession # AAG36711); Cry32Aa2 (Accession # GU063849); Cry32Ab1 (Accession # GU063850); Cry32Ba1 (Accession # BAB78601); Cry32Ca1 (Accession # BAB78602); Cry32Cb1 (Accession # KC156708); Cry32Da1 (Accession # BAB78603); Cry32Ea1 (Accession # GU324274); Cry32Ea2 (Accession # KC156686); Cry32Eb1 (Accession # KC156663); Cry32Fa1 (Accession # KC156656); Cry32Ga1 (Accession # KC156657); Cry32Ha1 (Accession # KC156661); Cry32Hb1 (Accession # KC156666); Cry32Ia1 (Accession # KC156667); Cry32Ja1 (Accession # KC156685); Cry32Ka1 (Accession # KC156688); Cry32La1 (Accession # KC156689); Cry32Ma1 (Accession # KC156690); Cry32Mb1 (Accession # KC156704); Cry32Na1 (Accession # KC156691); Cry32Oa1 (Accession # KC156703); Cry32Pa1 (Accession # KC156705); Cry32Qa1 (Accession # KC156706); Cry32Ra1 (Accession # KC156707); Cry32Sa1 (Accession # KC156709); Cry32Ta1 (Accession # KC156710); Cry32Ua1 (Accession # KC156655); Cry33Aa1 (Accession # AAL26871); Cry34Aa1 (Accession # AAG50341); Cry34Aa2 (Accession # AAK64560); Cry34Aa3 (Accession # AAT29032); Cry34Aa4 (Accession # AAT29030); Cry34Ab1 (Accession # AAG41671); Cry34Ac1 (Accession # AAG50118); Cry34Ac2 (Accession # AAK64562); Cry34Ac3 (Accession # AAT29029); Cry34Ba1 (Accession # AAK64565); Cry34Ba2 (Accession # AAT29033); Cry34Ba3 (Accession # AAT29031); Cry35Aa1 (Accession # AAG50342); Cry35Aa2 (Accession # AAK64561); Cry35Aa3 (Accession # AAT29028); Cry35Aa4 (Accession # AAT29025); Cry35Ab1 (Accession # AAG41672); Cry35Ab2 (Accession # AAK64563); Cry35Ab3 (Accession # AY536891); Cry35Ac1 (Accession # AAG50117); Cry35Ba1 (Accession # AAK64566); Cry35Ba2 (Accession # AAT29027); Cry35Ba3 (Accession # AAT29026); Cry36Aa1 (Accession # AAK64558); Cry37Aa1 (Accession # AAF76376); Cry38Aa1 (Accession # AAK64559); Cry39Aa1 (Accession # BAB72016); Cry40Aa1 (Accession # BAB72018); Cry40Ba1 (Accession # BAC77648); Cry40Ca1 (Accession # EU381045); Cry40Da1 (Accession # ACF15199); Cry41Aa1 (Accession # BAD35157); Cry41Ab1 (Accession # BAD35163); Cry41Ba1 (Accession # HM461871); Cry41Ba2 (Accession # ZP_04099652); Cry42Aa1 (Accession # BAD35166); Cry43Aa1 (Accession # BAD15301); Cry43Aa2 (Accession # BAD95474); Cry43Ba1 (Accession # BAD15303); Cry43Ca1 (Accession # KC156676); Cry43Cb1 (Accession # KC156695); Cry43Cc1 (Accession # KC156696); Cry43-like (Accession # BAD15305); Cry44Aa (Accession # BAD08532); Cry45Aa (Accession # BAD22577); Cry46Aa (Accession # BAC79010); Cry46Aa2 (Accession # BAG68906); Cry46Ab (Accession # BAD35170); Cry47Aa (Accession # AAY24695); Cry48Aa (Accession # CAJ18351); Cry48Aa2 (Accession # CAJ86545); Cry48Aa3 (Accession # CAJ86546); Cry48Ab (Accession # CAJ86548); Cry48Ab2 (Accession # CAJ86549); Cry49Aa (Accession # CAH56541); Cry49Aa2 (Accession # CAJ86541); Cry49Aa3 (Accession # CAJ86543); Cry49Aa4 (Accession # CAJ86544); Cry49Ab1 (Accession # CAJ86542); Cry50Aa1 (Accession # BAE86999); Cry50Ba1 (Accession # GU446675); Cry50Ba2 (Accession # GU446676); Cry51Aa1 (Accession # ABI14444); Cry51Aa2 (Accession # GU570697); Cry52Aa1 (Accession # EF613489); Cry52Ba1 (Accession # FJ361760); Cry53Aa1 (Accession # EF633476); Cry53Ab1 (Accession # FJ361759); Cry54Aa1 (Accession # ACA52194); Cry54Aa2 (Accession # GQ140349); Cry54Ba1 (Accession # GU446677); Cry55Aa1 (Accession # ABW88932); Cry54Ab1 (Accession # JQ916908); Cry55Aa2 (Accession # AAE33526); Cry56Aa1 (Accession # ACU57499); Cry56Aa2 (Accession # GQ483512); Cry56Aa3 (Accession # JX025567); Cry57Aa1 (Accession # ANC87261); Cry58Aa1 (Accession # ANC87260); Cry59Ba1 (Accession # JN790647); Cry59Aa1 (Accession # ACR43758); Cry60Aa1 (Accession # ACU24782); Cry60Aa2 (Accession # EAO57254); Cry60Aa3 (Accession # EEM99278); Cry60Ba1 (Accession # GU810818); Cry60Ba2 (Accession # EAO57253); Cry60Ba3 (Accession # EEM99279); Cry61Aa1 (Accession # HM035087); Cry61Aa2 (Accession # HM132125); Cry61Aa3 (Accession # EEM19308); Cry62Aa1 (Accession # HM054509); Cry63Aa1 (Accession # BAI44028); Cry64Aa1 (Accession # BAJ05397); Cry65Aa1 (Accession # HM461868); Cry65Aa2 (Accession # ZP_04123838); Cry66Aa1 (Accession # HM485581); Cry66Aa2 (Accession # ZP_04099945); Cry67Aa1 (Accession # HM485582); Cry67Aa2 (Accession # ZP_04148882); Cry68Aa1 (Accession # HQ113114); Cry69Aa1 (Accession # HQ401006); Cry69Aa2 (Accession # JQ821388); Cry69Ab1 (Accession # JN209957); Cry70Aa1 (Accession # JN646781); Cry70Ba1 (Accession # ADO51070); Cry70Bb1 (Accession # EEL67276); Cry71Aa1 (Accession # JX025568); Cry72Aa1 (Accession # JX025569).

Examples of δ-endotoxins also include but are not limited to Cry1A proteins of U.S. Pat. Nos. 5,880,275 and 7,858,849; a DIG-3 or DIG-11 toxin (N-terminal deletion of α-helix 1 and/or α-helix 2 variants of Cry proteins such as Cry1A) of U.S. Pat. Nos. 8,304,604 and 8,304,605, Cry1B of U.S. patent application Ser. No. 10/525,318; Cry1C of U.S. Pat. No. 6,033,874; Cry1F of U.S. Pat. Nos. 5,188,960, 6,218,188; Cry1A/F chimeras of U.S. Pat. Nos. 7,070,982; 6,962,705 and 6,713,063); a Cry2 protein such as Cry2Ab protein of U.S. Pat. No. 7,064,249); a Cry3A protein including but not limited to an engineered hybrid insecticidal protein (eHIP) created by fusing unique combinations of variable regions and conserved blocks of at least two different Cry proteins (US Patent Application Publication Number 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,378,499 and 7,462,760; a Cry9 protein such as such as members of the Cry9A, Cry9B, Cry9C, Cry9D, Cry9E, and Cry9F families; a Cry15 protein of Naimov, et al., (2008) Applied and Environmental Microbiology 74:7145-7151; a Cry22, a Cry34Ab 1 protein of U.S. Pat. Nos. 6,127,180, 6,624,145 and 6,340,593; a CryET33 and CryET34 protein of U.S. Pat. Nos. 6,248,535, 6,326,351, 6,399,330, 6,949,626, 7,385,107 and 7,504,229; a CryET33 and CryET34 homologs of US Patent Publication Number 2006/0191034, 2012/0278954, and PCT Publication Number WO 2012/139004; a Cry35Ab1 protein of U.S. Pat. Nos. 6,083,499, 6,548,291 and 6,340,593; a Cry46 protein, a Cry 51 protein, a Cry binary toxin; a TIC901 or related toxin; TIC807 of US 2008/0295207; ET29, ET37, TIC809, TIC810, TIC812, TIC127, TIC128 of PCT US 2006/033867; TIC1100, TIC 860, a TIC867, a TIC868, TIC869, and TIC836 of US Patent Publication Number 2016/0108428. AXMI-027, AXMI-036, and AXMI-038 of U.S. Pat. No. 8,236,757; AXMI-031, AXMI-039, AXMI-040, AXMI-049 of U.S. Pat. No. 7,923,602; AXMI-018, AXMI-020, and AXMI-021 of WO 2006/083891; AXMI-010 of WO 2005/038032; AXMI-003 of WO 2005/021585; AXMI-008 of US 2004/0250311; AXMI-006 of US 2004/0216186; AXMI-007 of US 2004/0210965; AXMI-009 of US 2004/0210964; AXMI-014 of US 2004/0197917; AXMI-004 of US 2004/0197916; AXMI-028 and AXMI-029 of WO 2006/119457; AXMI-007, AXMI-008, AXMI-0080rf2, AXMI-009, AXMI-014 and AXMI-004 of WO 2004/074462; AXMI-150 of U.S. Pat. No. 8,084,416; AXMI-205 of US20110023184; AXMI-011, AXMI-012, AXMI-013, AXMI-015, AXMI-019, AXMI-044, AXMI-037, AXMI-043, AXMI-033, AXMI-034, AXMI-022, AXMI-023, AXMI-041, AXMI-063, and AXMI-064 of US 2011/0263488; AXMI-R1 and related proteins of US 2010/0197592; AXMI221Z, AXMI222z, AXMI223z, AXMI224z and AXMI225z of WO 2011/103248; AXMI218, AXMI219, AXMI220, AXMI226, AXMI227, AXMI228, AXMI229, AXMI230, and AXMI231 of WO11/103247; AXMI-115, AXMI-113, AXMI-005, AXMI-163 and AXMI-184 of U.S. Pat. No. 8,334,431; AXMI-001, AXMI-002, AXMI-030, AXMI-035, and AXMI-045 of US 2010/0298211; AXMI-066 and AXMI-076 of US20090144852; AXMI128, AXMI130, AXMI131, AXMI133, AXMI140, AXMI141, AXMI142, AXMI143, AXMI144, AXMI146, AXMI148, AXMI149, AXMI152, AXMI153, AXMI154, AXMI155, AXMI156, AXMI157, AXMI158, AXMI162, AXMI165, AXMI166, AXMI167, AXMI168, AXMI169, AXMI170, AXMI171, AXMI172, AXMI173, AXMI174, AXMI175, AXMI176, AXMI177, AXMI178, AXMI179, AXMI180, AXMI181, AXMI182, AXMI185, AXMI186, AXMI187, AXMI188, AXMI189 of U.S. Pat. No. 8,318,900; AXMI079, AXMI080, AXMI081, AXMI082, AXMI091, AXMI092, AXMI096, AXMI097, AXMI098, AXMI099, AXMI100, AXMI101, AXMI102, AXMI103, AXMI104, AXMI107, AXMI108, AXMI109, AXMI110, AXMI111, AXMI112, AXMI114, AXMI116, AXMI117, AXMI118, AXMI119, AXMI120, AXMI121, AXMI122, AXMI123, AXMI124, AXMI1257, AXMI1268, AXMI127, AXMI129, AXMI164, AXMI151, AXMI161, AXMI183, AXMI132, AXMI138, AXMI137 of US 2010/0005543; and Cry proteins such as Cry1A and Cry3A having modified proteolytic sites of U.S. Pat. No. 8,319,019; a Cry1Ac, Cry2Aa and Cry1Ca toxin protein from Bacillus thuringiensis strain VBTS 2528 of US Patent Application Publication Number 2011/0064710, and an IP1B of PCT publication number WO 2016/061197. Other Cry proteins are well known to one skilled in the art (see, Crickmore, et al., “Bacillus thuringiensis toxin nomenclature” (2011), at lifesci.sussex.ac.uk/home/Neil_Crickmore/Bt/ which can be accessed on the world-wide web using the “www” prefix). The insecticidal activity of Cry proteins is well known to one skilled in the art (for review, see, van Frannkenhuyzen, (2009) J Invert. Path. 101:1-16). The use of Cry proteins as transgenic plant traits is well known to one skilled in the art and Cry-transgenic plants including but not limited to Cry1Ac, Cry1Ac+Cry2Ab, Cry1Ab, Cry1A.105, Cry1F, Cry1Fa2, Cry1F+Cry1Ac, Cry2Ab, Cry3A, mCry3A, Cry3Bb1, Cry34Ab1, Cry35Ab1, Vip3A, mCry3A, Cry9c and CBI-Bt have received regulatory approval (see, Sanahuja, (2011) Plant Biotech Journal 9:283-300 and the CERA (2010) GM Crop Database Center for Environmental Risk Assessment (CERA), ILSI Research Foundation, Washington D.C. at cera-gmc.org/index.php?action=gm_crop_database which can be accessed on the world-wide web using the “www” prefix). More than one pesticidal proteins well known to one skilled in the art can also be expressed in plants such as Vip3Ab & Cry1Fa (US2012/0317682), Cry1BE & Cry1F (US2012/0311746), Cry1CA & Cry1AB (US2012/0311745), Cry1F & CryCa (US2012/0317681), Cry1DA & Cry1BE (US2012/0331590), Cry1DA & Cry1Fa (US2012/0331589), Cry1AB & Cry1BE (US2012/0324606), and Cry1Fa & Cry2Aa, Cry1I or Cry1E (US2012/0324605)); Cry34Ab/35Ab and Cry6Aa (US20130167269); Cry34Ab/VCry35Ab & Cry3Aa (US20130167268); Cry3A and Cry1Ab or Vip3Aa (US20130116170); and Cry1F, Cry34Ab1, and Cry35Ab1 (PCT/US2010/060818). Pesticidal proteins also include insecticidal lipases including lipid acyl hydrolases of U.S. Pat. No. 7,491,869, and cholesterol oxidases such as from Streptomyces (Purcell et al. (1993) Biochem Biophys Res Commun 15:1406-1413). Pesticidal proteins also include VIP (vegetative insecticidal proteins) toxins of U.S. Pat. Nos. 5,877,012, 6,107,279, 6,137,033, 7,244,820, 7,615,686, and 8,237,020, and the like. Other VIP proteins are well known to one skilled in the art (see, lifesci.sussex.ac.uk/home/Neil_Crickmore/Bt/vip.html which can be accessed on the world-wide web using the “www” prefix). Pesticidal proteins also include toxin complex (TC) proteins, obtainable from organisms such as Xenorhabdus, Photorhabdus and Paenibacillus (see, U.S. Pat. Nos. 7,491,698 and 8,084,418). Some TC proteins have “stand alone” insecticidal activity and other TC proteins enhance the activity of the stand-alone toxins produced by the same given organism. The toxicity of a “stand-alone” TC protein (from Photorhabdus, Xenorhabdus or Paenibacillus, for example) can be enhanced by one or more TC protein “potentiators” derived from a source organism of a different genus. There are three main types of TC proteins. As referred to herein, Class A proteins (“Protein A”) are stand-alone toxins. Class B proteins (“Protein B”) and Class C proteins (“Protein C”) enhance the toxicity of Class A proteins. Examples of Class A proteins are TcbA, TcdA, XptA1 and XptA2. Examples of Class B proteins are TcaC, TcdB, XptB1Xb and XptC1Wi. Examples of Class C proteins are TccC, XptC1Xb and XptB1Wi. Pesticidal proteins also include spider, snake and scorpion venom proteins. Examples of spider venom peptides include but are not limited to lycotoxin-1 peptides and mutants thereof (U.S. Pat. No. 8,334,366).

Further transgenes that confer resistance to insects may down-regulate expression of target genes in insect pest species by interfering ribonucleic acid (RNA) molecules through RNA interference. PCT Publication WO 2007/074405 describes methods of inhibiting expression of target genes in invertebrate pests including Colorado potato beetle. PCT Publication WO 2005/110068 describes methods of inhibiting expression of target genes in invertebrate pests including in particular Western corn rootworm as a means to control insect infestation. Furthermore, PCT Publication WO 2009/091864 describes compositions and methods for the suppression of target genes from insect pest species including pests from the Lygus genus.

RNAi transgenes are provided for targeting the vacuolar ATPase H subunit, useful for controlling a coleopteran pest population and infestation are described in US Patent Application Publication 2012/0198586. PCT Publication WO 2012/055982 describes ribonucleic acid (RNA or double stranded RNA) that inhibits or down regulates the expression of a target gene that encodes: an insect ribosomal protein such as the ribosomal protein L19, the ribosomal protein L40 or the ribosomal protein S27A; an insect proteasome subunit such as the Rpn6 protein, the Pros 25, the Rpn2 protein, the proteasome beta 1 subunit protein or the Pros beta 2 protein; an insect β-coatomer of the COPI vesicle, the γ-coatomer of the COPI vesicle, the β′-coatomer protein or the ζ-coatomer of the COPI vesicle; an insect Tetraspanine 2 A protein which is a putative transmembrane domain protein; an insect protein belonging to the actin family such as Actin 5C; an insect ubiquitin-5E protein; an insect Sec23 protein which is a GTPase activator involved in intracellular protein transport; an insect crinkled protein which is an unconventional myosin which is involved in motor activity; an insect crooked neck protein which is involved in the regulation of nuclear alternative mRNA splicing; an insect vacuolar H+-ATPase G-subunit protein and an insect Tbp-1 such as Tat-binding protein. PCT publication WO 2007/035650 describes ribonucleic acid (RNA or double stranded RNA) that inhibits or down regulates the expression of a target gene that encodes Snf7. US Patent Application publication 2011/0054007 describes polynucleotide silencing elements targeting RPS10. PCT publication WO 2016/205445 describes polynucleotide silencing elements that reduce fecundity, with target polynucleotides, including NCLB, MAEL, BOULE, and VgR. U.S. Patent Application publication 2014/0275208 and US2015/0257389 describe polynucleotide silencing elements targeting RyanR and PAT3. PCT publications WO 2016/060911, WO 2016/060912, WO 2016/060913, and WO 2016/060914 describe polynucleotide silencing elements targeting COPI coatomer subunit nucleic acid molecules that confer resistance to Coleopteran and Hemipteran pests. US Patent Application Publications 2012/029750, US 20120297501, and 2012/0322660 describe interfering ribonucleic acids (RNA or double stranded RNA) that functions upon uptake by an insect pest species to down-regulate expression of a target gene in said insect pest, wherein the RNA comprises at least one silencing element wherein the silencing element is a region of double-stranded RNA comprising annealed complementary strands, one strand of which comprises or consists of a sequence of nucleotides which is at least partially complementary to a target nucleotide sequence within the target gene. US Patent Application Publication 2012/0164205 describe potential targets for interfering double stranded ribonucleic acids for inhibiting invertebrate pests including: a Chd3 Homologous Sequence, a Beta-Tubulin Homologous Sequence, a 40 kDa V-ATPase Homologous Sequence, a EFla Homologous Sequence, a 26S Proteosome Subunit p28 Homologous Sequence, a Juvenile Hormone Epoxide Hydrolase Homologous Sequence, a Swelling Dependent Chloride Channel Protein Homologous Sequence, a Glucose-6-Phosphate 1-Dehydrogenase Protein Homologous Sequence, an Act42A Protein Homologous Sequence, a ADP-Ribosylation Factor 1 Homologous Sequence, a Transcription Factor IIB Protein Homologous Sequence, a Chitinase Homologous Sequences, a Ubiquitin Conjugating Enzyme Homologous Sequence, a Glyceraldehyde-3-Phosphate Dehydrogenase Homologous Sequence, an Ubiquitin B Homologous Sequence, a Juvenile Hormone Esterase Homolog, and an Alpha Tubuliln Homologous Sequence.

### XI. Methods of Use

Methods disclosed herein comprise methods for controlling a plant insect pest (i.e., a Coleopteran plant pest, including a Diabrotica plant pest, such as, D. virgifera virgifera, D. barberi, D. virgifera zeae, D. speciosa, or D. undecimpunctata howardi). In one embodiment, the method comprises feeding or applying to a plant insect pest a composition comprising a silencing element and a MWLMV or JCSMV virus or modified MWLMV or JCSMV virus disclosed herein, wherein said silencing element, when ingested or contacted by a plant insect pest (i.e., but not limited to, a Coleopteran plant pest including a Diabrotica plant pest, such as, D. virgifera virgifera, D. barberi, D. virgifera zeae, D. speciosa, or D. undecimpunctata howardi), reduces the level of a target polynucleotide of the pest and thereby controls the pest and wherein the composition is has increased resistance to nuclease activity and midgut extract. The pest can be fed the silencing element in a variety of ways. For example, in an embodiment, the polynucleotide encoding the silencing element is introduced into a plant. As the plant pest feeds on the plant or part thereof expressing these sequences, the silencing element is delivered to the pest. When a silencing element and a MWLMV or JCSMV virus or modified MWLMV or JCSMV virus is delivered to the plant in this manner, it is recognized that the silencing element and a MWLMV or JCSMV virus or modified MWLMV or JCSMV virus may be expressed constitutively or alternatively, it may be produced in a stage-specific manner by employing the various inducible or tissue-preferred or developmentally regulated promoters that are discussed elsewhere herein. In specific embodiments, a silencing element and a MWLMV or JCSMV virus or modified MWLMV or JCSMV virus are expressed in the roots, stalk or stem, leaf including pedicel, xylem and phloem, fruit or reproductive tissue, silk, flowers and all parts therein or any combination thereof.

In another method, a composition comprising a silencing element and a MWLMV or JCSMV virus or modified MWLMV or JCSMV virus disclosed herein is applied to a plant. In such embodiments, a silencing element and a MWLMV or JCSMV virus or modified MWLMV or JCSMV virus may be formulated in an agronomically suitable and/or environmentally acceptable carrier, which is preferably, suitable for dispersal in fields. In addition, the carrier may also include compounds that increase the half-life of the composition. In specific embodiments, a composition comprising a silencing element and a MWLMV or JCSMV virus or modified MWLMV or JCSMV virus are formulated in such a manner such that it persists in the environment for a length of time sufficient to allow it to be delivered to a plant insect pest. In such embodiments, the composition can be applied to an area inhabited by a plant insect pest. In one embodiment, the composition is applied externally to a plant (i.e., by spraying a field) to protect the plant from pests.

In another embodiment, a method for the production of double stranded RNA is provided. The method comprises using a host cell, such as a bacteria cell, expressing a silencing element and a polynucleotide encoding a MWLMV or JCSMV virus or modified MWLMV or JCSMV virus, such as the polynucleotide sequences set forth in SEQ ID NOS.: 1-22, at large scale during fermentation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing embodiments have been described in some detail by way of illustration and example for purposes of clarity of understanding, certain changes and modifications may be practiced within the scope of the appended claims.

The following examples are offered by way of illustration and not by way of limitation.

### EXAMPLES

### Example 1. Expression of MWLMV RNA Genome and a Satellite Virus of MWLMV

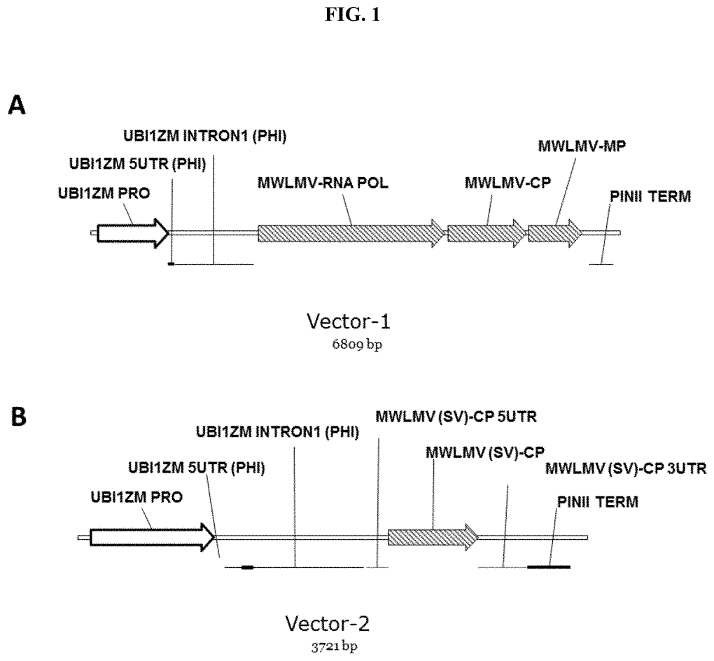

Sequences of MWLMV RNA genome and its satellite virus as well as each open-reading frame (ORF) sub-genome component is listed in Table 1, which includes: orf1 and orf2 encoding RNA directed-RNA polymerase (RNAP); orf3 encoding virus coat protein (CP); orf4 of movement protein (MP); and, orf of silencing suppressor protein (SP). These sequences or components of MWLMV were used for developing and designing different expression strategies for VIGS studies. The sequence of MWLMV RNA genome (SEQ ID NO: 1) was synthesized with BamHI and Hpa I cloning sites, and then inserted into a plant vector (FIG. 1, vector-1) under the control of maize UBI promoter. To express satellite virus of MWLMV, the sequence of satellite virus (sv) of MWLMV (SEQ ID NO: 7) was synthesized with Avr II and Hpa I cloning sites, and then inserted into a plant vector (FIG. 1, vector-2) under the control of maize UBI promoter. Satellite virus of MWLMV genome only has a single orf encoding satellite viral coat protein (sv-CP). The expression cassette of both constructs (vector-1 and vector-2) is shown in FIG. 1 and Table 2.

TABLE 1
MWLMV RNA genome and genes

| | Polynucleotide | | Amino Acid |
|---|---|---|---|
| Description | SEQ ID NO: | length nt | SEQ ID NO: |
| Maize white line mosaic | 1 | 4293 | n/a |
| virus, complete genome | | | |
| pre-readthrough region | 2 | 825 | 117 |
| of RNA directed-RNA | | | |
| polymerase | | | |
| RNA directed-RNA | 3 | 2394 | 118 |
| polymerase; p92; | | | |
| contains readthrough | | | |
| stop codon | | | |
| virus coat protein; | 4 | 999 | 119 |
| CP; ORF3 | | | |
| movement protein; | 5 | 684 | 120 |
| MP; ORF4 | | | |
| silencing suppressor | 6 | 417 | 121 |
| protein; ORF5 | | | |
| Satellite virus (“sv”) | 7 | 1168 | n/a |
| of maize white line | | | |
| mosaic virus | | | |
| virus coat protein ORF; | 8 | 657 | 122 |
| Satellite virus of maize | | | |
| white line mosaic virus | | | |

### Example 2. Modification of MVLMV Constructs for RNAi Applications

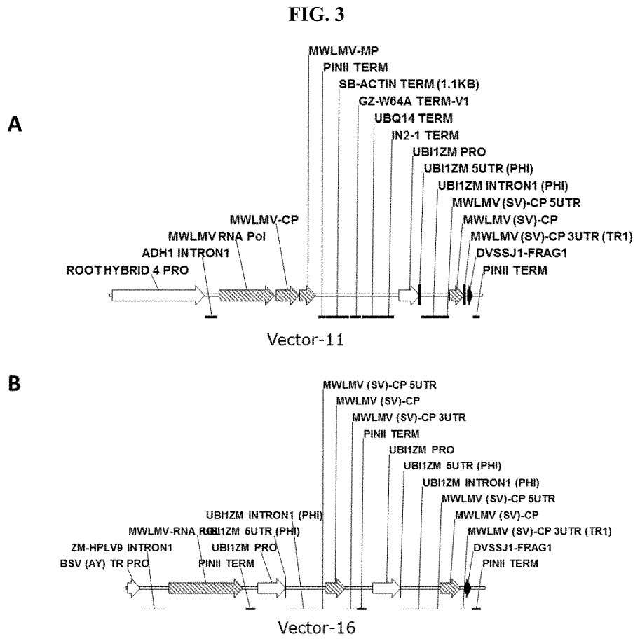

A series of constructs were designed in three groups (Table 2). Design group A was designed to express MWLMV with modification(s) (vector-4 to vector-9; SEQ ID NOS.: 17 and 149-150 with SEQ ID NO: 23 or 25 as an insert). Design group B contains two components including 1) the entire MWLMV genome driven by root specific promoter (root hybrid 4; RH4) and 2) the sv or sv with target genes (Vector 10 to 15; SEQ ID NOs: 18-19 and 151 and SEQ ID NO: 25 as an insert) under the control of Zm-UBI promoter. Design group C includes only RNAP of MWLMV, wild type sv-RNA and modified sv containing inserts of the gene of interest (GOI) (SEQ ID NOs: 23 or 25). Representative constructs of design groups A, B and C are illustrated in FIGS. 1-3. Design group A and B constructs were both designed to produce functional MWLMV and GOI targeting encapsidation inside the coat protein of the main virus or satellite virus. Design C was designed to produce only RNAP of MWLMV and a functional satellite virus plus the GOI targeting encapsidation inside the coat protein of satellite virus.

TABLE 2
Plant expression constructs containing MWLMV RNA genome, satellite virus, and target genes

| | | | | | | Gene of |
|---|---|---|---|---|---|---|
| | Construct | | MWLMV | Vector | Gene of | Interest |
| Construct ID | Description | Design | component | SEQ ID NO: | Interest | SEQ ID NO: |
| Vector-1 | UBI:MWLMV- | n/a | wild type | 15 | n/a | n/a |
| | RNA full | | MWLMV | | | |
| Vector-2 | UBI:MWLMV(SV) | n/a | wild type sv | 16 | n/a | n/a |
| Vector-4 | UBI:MWLMV- | A | modified | 17 | PDS | 23 |
| | MOD-PDS | | MWLMV | | | |
| | | | replace MP | | | |
| Vector-6 | UBI:MWLMV- | A | modified | 150 | ZsGreen | 25 |
| | MOD-ZsGreen | | MWLMV insert | | | |
| | | | at spacer-1 | | | |
| Vector-10 | RH4:MWLMV- | B | wild type | 18 | n/a | n/a |
| | UBI:sv | | MWLMV + sv | | | |
| Vector-14 | RH4:MWLMV- | B | wild type | 19 | ZsGreen | 25 |
| | UBI:sv-Zsgreen | | MWLMV + sv- | | | |
| | | | insert | | | |
| Vector-15 | FPM:MWLMV- | B | modified | 151 | n/a | n/a |
| | MOD-UBI:sv | | MWLM + sv- | | | |
| | | | wild type | | | |
| Vector-19 | BSV-RNAP-UBI- | C | MWLMV- | 20 | ZsGreen | 25 |
| | sv-root-UBI-sv- | | RNAP + sv-wild | | | |
| | Zsgreen | | type + sv-insert- | | | |
| | | | GOI | | | |

*Vector SEQ ID NO. represents the vector as described in the Construct Description column which includes the GOI, as also described separately in the GOI SEQ ID NO. column.

### Example 3. Quantification of RNA in MWLMV or Modified MWLMV or Cells by Quantigene

RNA levels were quantified using a customized Quantigene plex 2.0 assay panel (Affymetrix, Fremont, Calif., USA). Target RNA's within a sample homogenate hybridize to sequence-specific probes that were captured by their respective capture beads. Signal amplification was accomplished by consecutive hybridizations of a branched DNA pre-amplifier, amplifier and a biotinylated label probe. Detection and analysis were completed when the label probe was bound by Streptavidin-conjugated R-Phycoerythrin (SAPE). The SAPE fluorescent signal is measured using a Luminex MAGPIX (Luminex Corp., Austin, Tex., USA), which also determines the identity of the beads and their assigned sequence-specific probe. Capture beads and sequence-specific probes are all contained within the same reaction mix allowing for the multiplexing capability.

Hybridization and subsequent quantification were performed following the manufacturer's recommended procedure (see Quantigene Plex 2.0 Assay User Manual, Affymetrix). All reagents described below were purchased from Affymetrix. Plant extracts or control samples were diluted to an appropriate concentration and prepared using Affymetrix homogenizing solution (QG0517). Fluorescence was measured using the Luminex MAGPIX instrument with xPonent 4.2 software (Luminex). Luminescence was reported as Megpix fluorescence intensity (MFI units) and converted into picograms of viral genome/mg of fresh tissue (Tables 3 and 4). Quantification was done by extrapolation to the MFI of a standard curve made of in vitro transcripts (IVT) of each sequence. Final copy number was calculated based on the molecular weight of each IVT.

TABLE 3
Detection of viral RNA in transgenic plants

| Construct ID | MWI. | sv- MWL |
|---|---|---|
| Vector-1 plant 1 | >4.0 | 0.0 |
| Vector-1 plant 2 | >8.4 | 0.0 |
| Vector-1 plant 3 | >6.2 | 0.0 |
| Vector-1 plant 4 | >8.6 | 0.0 |
| Vector-1 plant 5 | >5.8 | 0.0 |
| Vector-1 plant 6 | 0.4 | 0.0 |
| Vector-2 plant 1 | 0.0 | 0.1 |
| Vector-2 plant 2 | 0.0 | 0.3 |
| Vector-2 plant 3 | 0.0 | 0.1 |
| Vector-2 plant 4 | 0.0 | 0.0 |
| Vector-2 plant 5 | 0.0 | 0.2 |
| Control (−) Non-transg | 0.0 | 0.0 |
| Control (+) wt infect | >4.4 | >14.0 |

*Individual events from each transgenic construct were tested for the presence of viral genome using Quantigene (QG). Results are presented in μg of viral genome/mg of fresh tissue. Samples with values above the dynamic range of QG method are marked as >X.

TABLE 4
Detection of viral RNA in plants infected
by vascular puncture inoculation

| inoculum | MWL | sv- MWL |
|---|---|---|
| MWLMV particles | >5.8 | 0.0 |
| MWLMV IVT | >3.5 | 0.0 |
| MWLMV particles + Sat part | >2.7 | >13.8 |
| MWLMV RNA + Sat RNA | >4.3 | >15.8 |
| MWLMV IVT + Sat IVT | >4.1 | >5.3 |
| MWLMV particles + Sat IVT | >3.5 | 0.6 |
| MWLMV particles + Sat-F3L IVT | >3.0 | 0.4 |
| MWLMV particles + Sat-F3L IVT | >3.9 | 1.1 |
| Control (+) wt infected reference | >2.0 | >9.7 |
| Control (−) non-infected | 0.0 | 0.0 |

*Plants infected after vascular puncture inoculation with several IVTs were tested for the presence of viral genome by Quantigene. Viral RNA was quantified using an IVT standard curve serial dilution and reported as pg of viral genome/mg of fresh tissue. Samples with values above the dynamic range of QG method are marked as >X.

### Example 4. Detection of Expression of MWLMV and Purification of Viral Particles from Infected or Transgenic Plants

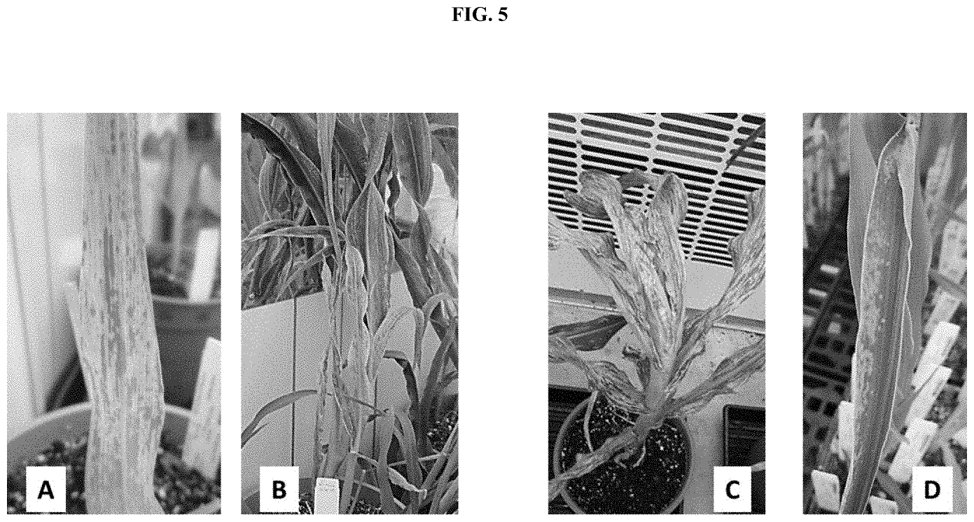

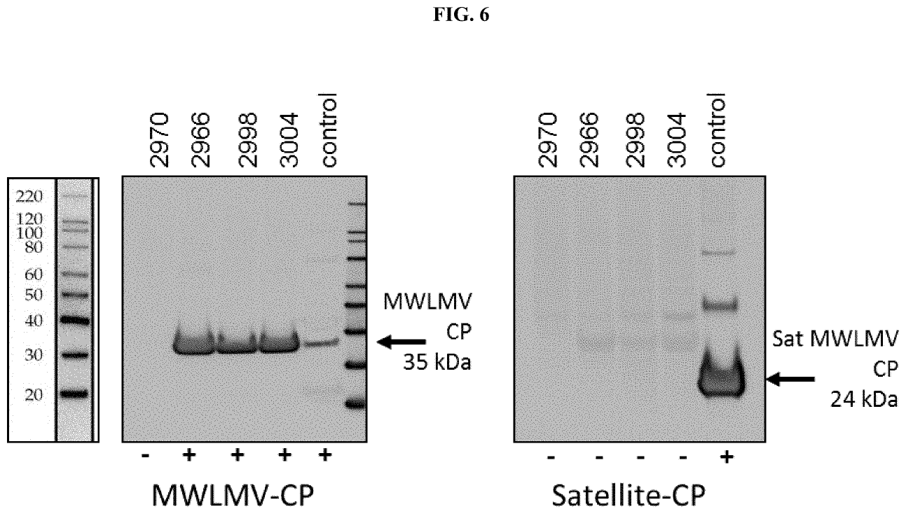

Viral protein expression was detected by Mass Spectrometry. ELISA was used to detect the coat protein of MWLMV and satellite MWLMV. Purification from infected, transgenic plants (FIG. 5) was done following De Zoeten protocol (de Zoeten, Amy et al. 1980). In brief, infected tissue was disrupted in neutral buffer and extracted with chloroform:butanol (1:1). The liquid phase was concentrated by ultracentrifugation (78,000×g). The enriched material in the resulting pellets was used to check for viral particle presence by Western blot (FIG. 6) and used as an inoculum for virus transmission.

Polyclonal antibodies to detect two different epitopes of both coat proteins were developed by GenScript (PolyExpress Silver Package). Samples from plants transgenic for MWLMV and from a control plant infected with MWLMV and satellite MWLMV were concentrated by ultracentrifugation to isolate viral particles. Expression of the viral genome was low in plant 2970 (Tables 3 and 5) and MWLMV-CP was not detected by western blot after ultra-concentration of virus particles (FIG. 6). No satellite-CP was detected in MWLMV transgenic plants (FIG. 6).

Mass spectrometry (MS) was used to detect MWLMV protein expression as described by Schacherer, L. J., et al. (2016). The maize leaves were harvested at approximately stage V5-V6 and ground after lyophilization. The extraction buffer used was 8M urea with 5 mM dithiothreitol (DTT) and 0.05% Tween 20. A total of 300 μL of extraction buffer was added per 10 mg leaf tissue, weighed into 1.2-mL micro titertubes (Quality Scientific Plastics, San Diego, Calif., USA). Both transgenic and null samples were run in triplicate. As shown in Table 5, peptides of four MWLMV proteins were positively detected in transgenic plants expressing MWLMV RNA genome but not in negative control. Also, transgenic plants expressing satellite viral genome showed positive detection of sv-CP peptide.

Detection of viral RNA and satellite RNA was done by Quantigene as described below. The expression level in Ti transgenic plants was measured by Quantigene using dsRNA prepared by in vitro transcription (IVT) as standard and compared to the expression of virus in infection. Plants transgenic for MWLMV under UBI promoter expressed >100 million copies of the viral genome (per mg of fresh leaf tissue), the detected expression level correlated with the symptom strength of the plant. Plants transgenic for satellite under UBI promoter expressed about a million copies/mg (FIG. 6). Satellite RNA levels are >10 fold higher in presence of MWLMV (compared T1-satellite plants versus T1-MWLMV x Satellite plants in FIG. 6). Transgenic driven viral replication results in similar levels of viral RNA in the infection (wt infection with MWLMV and satellite, FIG. 6). Also, the expression in transgenic plants was compared to the expression of a gene of interest under the same UBI promoter. The final copy numbers obtained in MWLMV transgenic plants resulted >10 fold higher than regular UBI-driven expression of a gene of interest, Seq No. 31 (Hu, Richtman et al. 2016).

TABLE 5
MS detection of MWLMV expression in transgenic maize

| | Protein | | | |
|---|---|---|---|---|
| | MWLMV- | MWLMV- | MWLMV- | MWLMV |
| | REP | CP | MP | Sat-CP |
| | Peptide SEQ ID NO: | | | |
| | SEQ ID | SEQ ID | SEQ ID | SEQ ID |
| | NO: 126 | NO: 127 | NO: 128 | NO: 129 |
| Construct | | | | |
| Vector-1 | 21.70 | 319.88 | 359.27 | n/d |
| Vector-1 | 0.87 | 106.16 | 107.20 | n/d |
| Vector-1 | 5.47 | 281.18 | 215.56 | n/d |
| Vector-1 | 3.39 | 143.18 | 87.59 | n/d |
| Vector-1 | 5.81 | 372.67 | 371.09 | n/d |
| Vector-2 | n/d | n/d | n/d | 0.25 |
| Vector-2 | n/d | n/d | n/d | 0.05 |
| Vector-2 | n/d | n/d | n/d | 0.05 |
| Vector-2 | n/d | n/d | n/d | 0.21 |
| Vector-2 | n/d | n/d | n/d | 0.06 |
| non-transgenic | 0.00 | 0.00 | 0.00 | 0.00 |
| Control (+) | 100.00 | 100.00 | 100.00 | 100.00 |
| wt infect | | | | |

*Extracts from independent events of transgenic plants for 2 vectors were analyzed by mass spectrometry to detect specific peptides from viral proteins. Peptide detection levels are expressed in relation to the levels detected in a wild type infected positive control (considered as 100%). n/d, not detected.

### Example 5. In Vitro Transcription of MWLMV and Satellite MWLMV RNA for Viral Infection

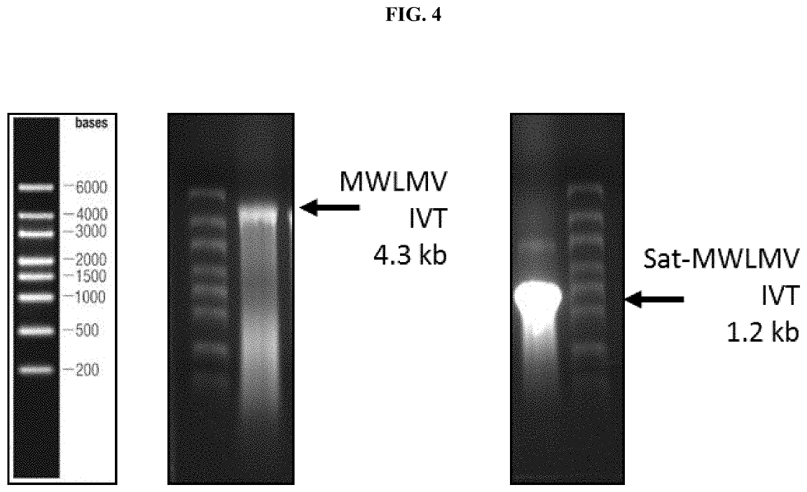

Templates for in vitro transcription (IVT) were amplified by PCR using plasmids containing SEQ ID NO: 1 (MWLMV) and a plasmid containing SEQ ID NO: 7 (sv MWLMV). The forward primer included a T7 promoter sequence to drive the transcription. PCR reaction was done using OneTaq® Quick-Load® 2X Master Mix with GC Buffer (New England Biolabs, M0487). Products of expected sizes were cleaned using QIAquick Gel Extraction Kit (Qiagen, 28704). IVT reactions were done following MEGAscript® Kit protocol (Life Technologies, AM1330). IVT products (single stranded RNAs) were visualized by denaturing agarose electrophoresis (FIG. 4). IVT products were used to inoculate seeds in transmission experiments (Table 4).

### Example 6. MWLMV Infection

Vascular puncture inoculation of ungerminated seed was used to infect corn plants following the protocol reported by Louie et al., 1995, Phytopathology. A tattoo multi-pin needle was used to mechanically inoculate 1-2 μL of viral preparations in the embryo side of the seeds. Inoculated seeds were planted directly into the soil and maintained inside growth chamber. Both Plants inoculated with MWLMV virions extracted from transgenic plants or inoculated with IVTs of MWLMV and satellite MWLMV developed the characteristic symptoms of MWLMV infection after 10 days of inoculation (FIG. 5).

### Example 7. Agrobacterium-Mediated Transformation of Maize

For Agrobacterium-mediated maize transformation with the disclosed polynucleotide constructs comprising a silencing element as disclosed herein, the method of Zhao can be employed (U.S. Pat. No. 5,981,840 and International Patent Publication Number WO 1998/32326, the contents of which are hereby incorporated by reference). Briefly, immature embryos are isolated from maize and the embryos are contacted with an Agrobacterium suspension, where the bacteria are capable of transferring the desired disclosed polynucleotide constructs comprising a silencing element as disclosed herein to at least one cell of at least one of the immature embryos (step 1: the infection step). In this step, the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period, an optional resting step can be contemplated. In this resting step, the embryos are incubated in the presence of at least one antibiotic known to inhibit Agrobacterium growth without a plant transformant selective agent (step 3: resting step). The immature embryos are cultured on solid medium with antibiotic, but without a selecting agent, for Agrobacterium elimination and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus can then be regenerated into plants (step 5: the regeneration step), and calli grown on selective medium are cultured on solid medium to regenerate the plants.

### Example 8. Expression of Viral Elements in Maize

The Viral genome or elements were expressed in a maize plant using the transformation techniques in Example 7.

Maize plants were transformed with plasmids containing genes listed in Table 1 or 2, and plants expressing the entire viral RNA genome or elements were transplanted from 272V plates into greenhouse flats containing Fafard Superfine potting mix. Approximately 10 to 14 days after transplant, plants (now at growth stage V2-V3) were transplanted into three pots containing Fafard Superfine potting mix. Transgenic plants were transferred into a larger pot and observed for MWLMV systemic symptoms (FIG. 4). Samples were collected at different stages or from different tissues for viral RNA detection (See Table 3 and FIG. 6) and/or protein expression analyses (Table 5) or MWLMV infection confirmation.

TABLE 6
Characterization of transgenic plants
with modified versions of MWLMV vector.

| | | | MWLMV | |
|---|---|---|---|---|
| Construct | | Viral | Systemic | Protein |
| ID | Description | RNA μg/mg | Symptoms | MWL-CP |
| — | Non-transgenic | 0.0 | NO | NO |
| — | Infected with | >8.53 | YES | YES |
| | wild type | | | |
| | MWLMV | | | |
| Vector-10 | RH4:MWLMV- | >4.13 | YES | YES |
| | UBI:sv | | | |
| Vector-15 | FPM:MWLMV- | 0.84 | NO | NO |
| | MOD-UBI:sv | | | |
| Vector-6 | UBI:MWLMV- | >5.31 | YES | YES |
| | MOD-ZsGreen | | | |

Plants from each construct were tested for the presence of viral genome using Quantigene. Results are presented in picogram of viral genome/mg of fresh tissue (average of 10 plants). Samples with values above the dynamic range of QG method are marked as >X. Coat protein expression was detected by Western blot and Mass Spec analyses. A construct expressing the wild type sequence as well as plants infected with wild type virus (ATCC® PV489™) were used as a reference. As negative controls, a modified vector with punctual mutations that abolish viral replication (vector-14) and non-transgenic plants are shown.

For constructs containing a Western Corn Rootworm (WCRW) target gene fragment (a silencing element, SEQ ID NO: 24), at 14 days post greenhouse send date, Ti plants are infested with 200 eggs of WCRW per plant. A second infestation of 200 eggs WCRW per plant is done 7 days after the first infestation and scoring is performed at 14 days after the second infestation. 21 days post-infestation, plants are scored using CRWNIS.

### Example 9. Characterization of Transgenic Plants Expressing Zsgreen Marker in the spacer-1 of MWLMV

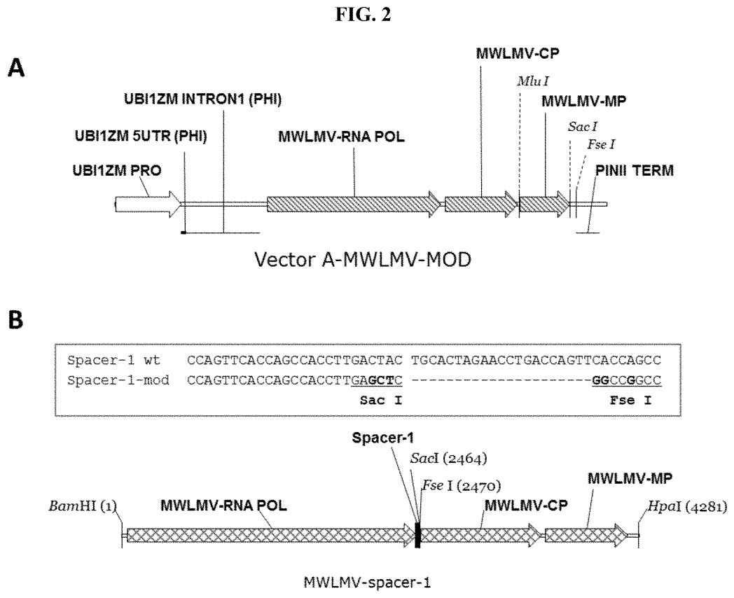

Modified MWLMV vectors showed different phenotypes (MWLMV systemic symptoms) and expression patterns as indicated in Table 3, 4, 5 and 6. Most of the plants transformed with constructs showed no infectious symptoms indicating that changes to the RNA genome resulted in no viral replication, which was supported by low expression of RNA and no detection of coat protein. These constructs included three restriction sites (three SNPs per site) that were designed for cloning a gene of interest (GOI), and/or MWLMV-CP/MP were replaced with a marker in Design A (See Table 2 and FIG. 2). However, transgenic plants containing an insertion of Zsgreen in spacer-1 region showed MWLMV systemic symptoms and CP expression. Further analyses of individual transgenic lines demonstrated that spacer-1 region can be explored for inserting a polynucleotide sequence expressing silencing element targeting a GOI as indicated in Table 7.

Extracts from symptomatic tissue (Vector-6) were treated with nucleases to remove non-encapsidated nucleic acids. Total RNA was extracted, and RT-PCR amplification of the flanking insert in cloning sites of Spacer-1-mod (FIG. 2) resulted in products of different sizes. A total of 25 plants (individual transgenic events) were analyzed. Samples with inserts >40 bp are shown. Spacer-1-mod and vector-16 are shown as references. Insert size includes sequence from the 5′ end of SacI to the 3′ end of FseI.

TABLE 7
Virus-like particles produced in transgenic
plants (vector-6) contain variable sizes of
remaining ZsGreen insert in the viral genome.

| | | insert | | SEQ |
|---|---|---|---|---|
| | insert 5′ | 3′end | | ID |
| Product | end SaCI | FseI | insert | NO: |
| spacer-1- | CACCAGCCACCT | GGCCGGCC | 14 b | 131 |
| mod | TGAGCTC | | | |
| vector-16 | CACCAGCCACCT | GGCCGGCC | 728 b | 132 |
| | TGAGCTC | | | |
| plant 1 | CA | GGCCGGCC | 114 | 133 |
| plant 2 | CACCAGCCACCT | GGCCGGCC | 86 b | 134 |
| | TGA | | | |
| plant 3 | CACCAGCCACCT | GACGGGCN | 85 b | 135 |
| | TGA | | | |
| plant 4 | CACCAGCCACCT | GGCCAGCC | 83 b | 136 |
| | TGA | | | |
| plant 5 | CACCTGACCCCT | GGCCGGCC | 41 b | 137 |
| | TGA | | | |
| plant 6 | CACCCTGACCCT | ACTCGGAT | 88 b | 138 |
| | TGA | | | |
| plant 7 | CA | GGCCGGCC | 115-11 b | 139 |

### Example 10. Comparison of RNA Genomes of MWLMV and JCSMV

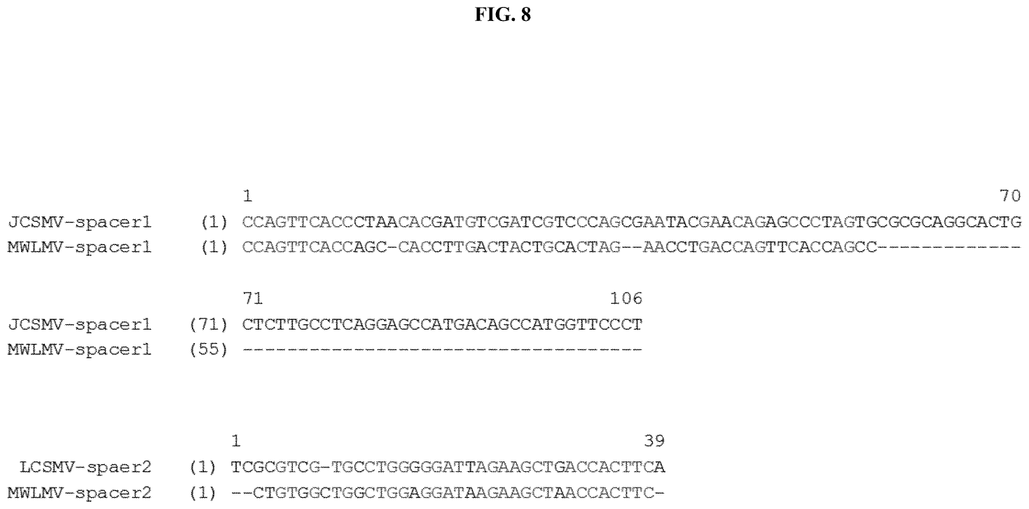

Johnsongrass chlorotic stripe mosaic virus (JCSMV) is the closest relative of MWLMV reported to this date. It was originally isolated from stunt johnsongrass plants (Sorghum halepense) showing chlorotic stripes (Izadpanah, K. 1998). Its genome consists of linear single-stranded RNA (ssRNA) 4421 nt long [NCBI GenBank (AJ557804.1), Table-8](SEQ ID NO: 9), encoding 5 proteins in the same order and arrangement as MWLMV. Open Reading Frame (ORF) 1 (SEQ ID NO: 10) codes for a pre-readthrough of the RNA directed-RNA polymerase (Pre-RNAP) with a predicted molecular weight of 30.5 kDa. ORF 2 (SEQ ID NO: 11) codes for the viral replicase, RNA directed-RNA polymerase (RNAP) predicted to be 89.2 kDa. Pre-RNAP and RNAP are involved in replication of viral genome. ORF 3 (SEQ ID NO: 12) codes for the viral coat protein (CP) of 39 kDa. ORF 4 (SEQ ID NO: 13) encodes a movement protein (MP) of 23.8 kDa predicted to transport viral genome inside the plant. ORF 5 (SEQ ID NO: 14) codes for a small protein of 15.3 kDa, a putative viral suppressor of RNA silencing (SP). Sequence comparison of the MWLMV and JCSMV genomes revealed that spacer-1 region (FIG. 8) showed the least homology (30.2%) between the two RNA genomes, and supported the interpretation that this region may be less conserved and can be explored for target GOI insertion (Table 9).

TABLE 8
Johnsongrass chlorotic stripe mosaic
virus (JCSMV) RNA genome and genes

| | | | Amino |
|---|---|---|---|
| | Polynucleotide | length | Acid SEQ |
| Description | SEQ ID NO: | nt | ID NO: |
| Johnsongrass chlorotic | 9 | 4421 | n/a |
| stripe mosaic virus | | | |
| (JCSMV), complete genome | | | |
| JCSMV pre-readthrough | 10 | 819 | 140 |
| region of RNA directed- | | | |
| RNA polymerase | | | |
| JCSMV RNA directed-RNA | 11 | 2388 | 141 |
| polymerase; p92; contains | | | |
| readthrough stop codon | | | |
| JCSMV virus coat protein; | 12 | 1095 | 142 |
| CP; ORF3 | | | |
| JCSMV movement protein; | 13 | 654 | 143 |
| MP; ORF4 | | | |
| JCSMV silencing suppressor | 14 | 420 | 144 |
| protein; ORF5 | | | |

TABLE 9
Comparison of MWLMV and JCSMV RNA
genome and their sequence identity

| Size (nt) | MWLMV | JCSMV | % identity |
|---|---|---|---|
| RNA genome | 4293 | 4421 | 63.6 |
| 5utr | 40 | 44 | 55.6 |
| ORF-1 | 825 | 819 | 63.4 |
| ORF-2 | 2394 | 2388 | 70.2 |
| SPACER-1 | 54 | 106 | 30.2 |
| ORF-3-CP | 999 | 1095 | 46.1 |
| SPACER-2 | 36 | 38 | 71.8 |
| ORF-4-MP | 684 | 654 | 68.3 |
| ORF-5-SP | 417 | 420 | 78.8 |
| 3utr | 86 | 96 | 38.3 |

### Example 11. Design and Characterization of Transgenic Plants Expressing Target RNA in the Spacer-1 of MWLMV

MWLMV vectors containing expressing cassette (FIG. 9; 83 bp or 463 bp inserts between Sac I and Fse I) in the spacer-1 region were designed and tested in transgenic maize plants. The inserted target (DVSSJ1, SEQ ID NO: 24) has been demonstrated insecticidal activity against western corn rootworm (Xu Hu et. al. 2016). Transgenic plants showed MWLMV systemic symptoms and CP expression in most of the transgenic plants (FIG. 10). Further analyses of individual transgenic lines demonstrated that DvSSJ1 transcripts and viral RNA were expressed as indicated in Table 10.

TABLE 10
Detection of viral RNA and transgenic target in transgenic plants

| dsRNA | Zma- | | Dvssj1- | ssRNA | Zma- | | Dvssj1- |
|---|---|---|---|---|---|---|---|
| vector | actin | MWL | frag3 | vector | actin | MWL | frag3 |
| 342885332 | 37 | 24209 | 10 | 343095116 | 94 | 22869 | 67 |
| 342885354 | 141 | 13425 | 418 | 343095118 | 37 | 1626 | 5 |
| 342885355 | 115 | 22066 | 153 | 343095122 | 33 | 28125 | 44 |
| 342885368 | 44 | 20944 | 485 | 343095129 | 37 | 29763 | 145 |
| 342885369 | 28 | 35811 | 810 | 343095131 | 58 | 20598 | 50 |
| 342885385 | 45 | 41374 | 459 | 343095141 | 52 | 31131 | 67 |
| 342885401 | 30 | 24708 | 69 | 343095193 | 95 | 27457 | 262 |
| 342885407 | 37 | 23531 | 2 | 343095146 | 60 | 43117 | 97 |
| 342885410 | 28 | 33086 | 37 | 343095198 | 35 | 39457 | 94 |
| 342885408 | 54 | 2161 | 19 | 343095201 | 32 | 30168 | 7 |
| HC69 | 57 | 10 | 4 | HC69 | 57 | 10 | 4 |
| HC69 | 66 | 13 | 4 | HC69 | 66 | 13 | 4 |

* Raw fluorescence readings of transgenic plants were compared to non-transgenic control (HC69). Maize actin gene (Zma-actin) were included as internal control to compare with viral RNA (MWL) and transgenic transcript (Dvssj1 frag3).

1. An isolated polynucleotide comprising a polynucleotide encoding a silencing element and a polynucleotide encoding a MWLMV, a JCSMV, a virus derived from a MWLMV, a virus derived from a JCSMV, or a MWLMV satellite, wherein the silencing element, when ingested by a plant pest, controls the plant pest.

2. The isolated polynucleotide of claim 1, wherein the MWLMV comprises of a nucleotide sequence of at least 90% sequence identity to SEQ ID NO: 4, encoding a MWLMV coat protein.

3. The isolated polynucleotide of claim 1, wherein the MWLMV satellite comprises of a nucleotide sequence of at least 90% sequence identity to SEQ ID NO: 8, encoding a satellite MWLMV coat protein.

4. The isolated polynucleotide of claim 1, wherein the JCSMV comprises of a nucleotide sequence of at least 90% sequence identity to SEQ ID NO: 12, encoding a JCSMV coat protein.

5. The isolated polynucleotide of claim 2, further comprising a polynucleotide encoding a MWLMV movement peptide comprising a nucleotide sequence of at least 90% sequence identity to SEQ ID NO: 5.

6. The isolated polynucleotide of claim 2, further comprising a polynucleotide encoding a MWLMV RNA directed RNA polymerase comprising a nucleotide sequence of at least 90% sequence identity to SEQ ID NO: 3.

7. The isolated polynucleotide of claim 1, further comprising a polynucleotide encoding a MWLMV movement peptide comprising a nucleotide sequence of at least 90% sequence identity to SEQ ID NO: 5.

8. The isolated polynucleotide of claim 4, further comprising a polynucleotide encoding a JCSMV RNA directed RNA polymerase comprising a nucleotide sequence of at least 90% sequence identity to SEQ ID NO: 11.

9. The isolated polynucleotide of claim 1, wherein the silencing element comprises at least 21, at least 50, at least 100, or at least 200 nucleotides.

10. The isolated polynucleotide of claim 1, wherein the silencing element comprises at least two different target polynucleotides.

11. The isolated polynucleotide of claim 1, wherein the plant pest is a Coleopteran, Lepidopteran, or Hemipteran plant pest.

12. The isolated polynucleotide of claim 11, wherein the Coleopteran plant pest is a Diabrotica plant pest.

13. The isolated polynucleotide of claim 11, wherein the Lepidopteran plant pest is a Spodoptera frugiperda plant pest.

14. The isolated polynucleotide of claim 1, wherein the silencing element expresses as a double stranded RNA.

15. The isolated polynucleotide of claim 14, wherein each strand of the double stranded RNA comprises at least 21, at least 50, at least 100, or at least 200 nucleotides.

16. The isolated polynucleotide of claim 1, wherein the silencing element expresses as a hairpin RNA.

17. A DNA construct comprising the polynucleotide of claim 1.

18. An expression construct comprising the DNA construct of claim 17.

19. (canceled)

20. A host cell comprising the expression cassette of claim 18.

21. The host cell of claim 20, wherein the host cell is a bacterial cell.

22. (canceled)

23. The host cell of claim 20, wherein the expression construct comprises a heterologous promoter operably linked to the DNA construct of claim 17.

24. A DNA construct comprising a polynucleotide encoding a silencing element and a MWLMV or a JCSMV RNA dependent RNA polymerase, wherein the silencing element, when ingested by a plant pest, controls the plant pest.

25. The DNA construct of claim 24, wherein the RNA dependent RNA polymerase comprises of a nucleotide sequence of at least 90% sequence identity to SEQ ID NO: 3.

26. The DNA construct of claim 24, wherein the RNA dependent RNA polymerase comprises a nucleotide sequence of at least 90% sequence identity to SEQ ID NO: 11.

27. The DNA construct of claim 24, further comprising a polynucleotide sequence having at least 90% sequence identity to SEQ ID NOS.: 1, 2, 4-8, 10, or 13-14.

28. An expression cassette comprising the DNA construct of claim 24.

29. (canceled)

30. A host cell comprising the expression cassette of claim 28.

31. The host cell of claim 30, wherein the host cell is a bacterial cell.

32. (canceled)

33. The host cell of claim 30, wherein the host cell is a plant cell.

34. (canceled)

35. The DNA construct of claim 24, wherein the silencing element comprises at least 21, at least 50, at least 100, or at least 200 nucleotides.

36. (canceled)

37. The DNA construct of claim 24, wherein the silencing element expresses as a double stranded RNA.

38. The DNA construct of claim 24, wherein the silencing element expresses as a hairpin RNA.

39. The DNA construct of claim 24, wherein the plant pest is a Coleopteran, Lepidopteran, or Hemipteran plant pest.

40. (canceled)

41. (canceled)

42. A plant cell having stably incorporated into its genome a heterologous polynucleotide comprising a polynucleotide encoding a silencing element and a MWLMV, a JCSMV, a virus derived from a MWLMV, a virus derived from a JCSMV, or a MWLMV satellite, wherein the silencing element, when ingested by a plant pest, controls the plant pest.

43. The plant cell of claim 42, wherein the MWLMV comprises of a nucleotide sequence having at least 90% sequence identity to SEQ ID NO: 4, encoding a MWLMV coat protein.

44. The plant cell of claim 42, wherein the MWLMV satellite comprises a nucleotide sequence of at least 90% sequence identity to SEQ ID NO: 8, encoding a satellite MWLMV coat protein.

45. The plant cell of claim 42, wherein the JCSMV comprises a nucleotide sequence of at least 90% sequence identity to SEQ ID NO: 12, encoding a JCSMV coat protein.

46. The plant cell of claim 42, further comprising a polynucleotide encoding a MWLMV movement peptide comprising a nucleotide sequence of at least 90% sequence identity to SEQ ID NO: 5.

47. The plant cell of claim 42, further comprising a polynucleotide encoding a MWLMV RNA dependent RNA polymerase comprising a nucleotide sequence of at least 90% sequence identity to SEQ ID NO: 3.

48.-52. (canceled)

53. The plant cell of claim 42, wherein the silencing element expresses as a double stranded RNA.

54. (canceled)

55. The plant cell of claim 42, wherein the silencing element expresses as a hairpin RNA.

56. The plant cell of claim 42, wherein the plant cell is from a monocot.

57. (canceled)

58. The plant cell of claim 42, wherein the plant cell is from a dicot.

59. (canceled)

60. A method for controlling a plant insect pest comprising feeding to a plant insect pest a composition comprising a heterologous polynucleotide encoding a silencing element and a a MWLMV, a JCSMV, a virus derived from a MWLMV, a virus derived from a JCSMV, or a MWLMV satellite, wherein the silencing element, when ingested by a plant pest, controls the plant pest and wherein the composition has increased resistance to nuclease activity and midgut extract.

61. The method of claim 62, wherein the MWLMV comprises a nucleotide sequence of at least 90% sequence identity to SEQ ID NO: 4, encoding a MWLMV coat protein.

62. The method of claim 62, wherein the MWLMV satellite comprises a nucleotide sequence of at least 90% sequence identity to SEQ ID NO: 8, encoding a satellite MWLMV coat protein.

63. The method of claim 62, wherein the JCSMV comprises a nucleotide sequence of at least 90% sequence identity to SEQ ID NO: 12, encoding a JCSMV coat protein.

64. The method of claim 62, further comprising a polynucleotide encoding a MWLMV movement peptide comprising a nucleotide sequence having at least 90% sequence identity to SEQ ID NO: 120.

65. The method of claim 62, further comprising a polynucleotide encoding a MWLMV RNA dependent RNA polymerase comprising a nucleotide sequence of at least 90% sequence identity to SEQ ID NO: 3.

66. The method of claim 62, wherein the silencing element comprises at least 21, at least 50, at least 100, or at least 200 nucleotides.

67.-112. (canceled)
